(19) United States
(12) Patent Application Publication
(10) Pub. No.: US 2007/0105874 A1
(43) Pub. Date: May 10, 2007

(54) Anti-Tumor Methods Using Multi Drug Resistance Independent Synthetic HSP90 Inhibitors

(75) Inventors: Hong Zhang, San Diego, CA (US); Francis J. Burrows, Solana Beach, CA (US)

Correspondence Address: WILSON SONSINI GOODRICH & ROSATI, 650 PAGE MILL ROAD, PALO ALTO, CA, 94304-1050, US

(73) Assignee: Conforma Therapeutics Corporation, San Diego, CA (US)

(21) Appl. No.: 11/535,046

(22) Filed: Sep. 25, 2006

Related U.S. Application Data
(60) Provisional application No. 60/720,273, filed on Sep. 23, 2005.

(51) Int. Cl. A61K 31/52; A61K 31/519

(52) U.S. Cl. 514/259.3; 514/263.4; 514/261.1; 514/259.31; 514/265.1

(57) ABSTRACT

The present invention provides a method of treating an individual having an HSP90 mediated disorder comprising administering to said individual a pharmaceutical composition comprising a pharmaceutically effective amount of a synthetic heterocyclic HSP90 inhibitor, wherein the activity of the HSP90 inhibitor is substantially independent of multi drug resistance. In one embodiment, the activity of the HSP90 inhibitor is substantially independent of P-gp and MRP expression.

### RELATED APPLICATIONS

This application claims the benefit of U.S. Provisional Application No. 60/720,273 filed Sep. 23, 2005, which is herein incorporated by reference in its entirety. The present application also relates to compounds and methods disclosed in U.S. Applications: 60/591,467, filed on Jul. 26, 2004, 60/555,567, filed on Mar. 22, 2004, Application Ser. No. 60/666,899, filed on Mar. 30, 2005, Ser. No. 10/946,645 filed Sep. 20, 2004 (now Publication No. 20050113340); Ser. No. 10/946,637 filed Sep. 20, 2004 (now Publication No. 2005-119282), Ser. No. 10/945,851 filed Sep. 20, 2004 (now Publication No. 20050107343) Ser. No. 10/946,628 filed Sep. 20, 2004 (now Publication No. 20050113339) and Ser. No. 11/277,918 filed Mar. 29, 2006. This application further relates to International Applications PCT/US02/35069, filed Oct. 30, 2002, (now Publication No. WO03/37860) and PCT/US06/11846.

The contents of these applications are herein incorporated by reference in their entirety.

### BACKGROUND OF THE INVENTION

HSP90s are ubiquitous chaperone proteins that are involved in folding, activation and assembly of a wide range of proteins, including key proteins involved in signal transduction, cell cycle control and transcriptional regulation. Researchers have reported that HSP90 chaperone proteins are associated with important signaling proteins, such as steroid hormone receptors and protein kinases, including, e.g., Raf-1, EGFR, v-Src family kinases, Cdk4, and ErbB-2 (Buchner J. TIBS 1999, 24, 136-141; Stepanova, L. et al. Genes Dev. 1996, 10, 1491-502; Dai, K. et al. J. Biol. Chem. 1996, 271, 22030-4). Studies further indicate that certain co-chaperones, e.g., HSP7O, p60/Hop/Sti1, Hip, Bag1, HSP40/Hdj2/Hsj1, inmmnunophilins, p23, and p50, may assist HSP90 in its function (see, e.g., Caplan, A. Trends in Cell Biol. 1999, 9, 262-68).

HSP90 possesses a binding pocket at its N-terminus. This pocket is highly conserved and has weak homology to the ATP-binding site of DNA gyrase (Stebbins, C. et al., supra; Grenert, J. P. et al. J. Biol. Chem. 1997, 272, 23843-50). Further, ATP and ADP have both been shown to bind this pocket with low affinity and to have weak ATPase activity (Proromou, C. et al. Cell 1997, 90, 65-75; Panaretou, B. et al. EMBO J 1998, 17, 4829-36). In vitro and in vivo studies have demonstrated that occupancy of this N-terminal pocket by ansamycins and other HSP90 inhibitors alters HSP90 function and inhibits protein folding. At high concentrations, ansamycins and other HSP90 inhibitors have been shown to prevent binding of protein substrates to HSP90 (Scheibel, T. H. et al. Proc. Natl Acad. Sci. USA 1999, 96, 1297-302; Schulte, T. W. et al. J. Biol. Chem. 1995, 270, 24585-8; Whitesell, L., et al. Proc. Natl. Acad. Sci. USA 1994, 91, 8324-8328). HSP90 inhibitors, e.g. ansamycins, have also been demonstrated to inhibit the ATP-dependent release of chaperone-associated protein substrates (Schneider, C. L. et al. Proc. Natl. Acad. Sci, USA 1996, 93, 14536-41; Sepp-Lorenzino et al. J Biol. Chem. 1995, 270, 16580-16587). In either event, the substrates are degraded by an ubiquitin-dependent process in the proteasome (Schneider, C. L., supra; Sepp-Lorenzino, L., et al., J Biol. Chem. 1995, 270, 16580-16587; Whitesell, L. et al. Proc. Natl. Acad. Sci. USA 1994, 91, 8324-8328).

HSP90 substrate destabilization occurs in tumor and non-transformed cells alike and has been shown to be especially effective on a subset of signaling regulators, e.g., Raf (Schulte, T. W. et al. Biochem. Biophys. Res. Commun. 1997, 239, 655-9; Schulte, T. W., et al. J Biol. Chem. 1995, 270, 24585-8), nuclear steroid receptors (Segnitz, B.; U. Gebring J. Biol. Chem. 1997, 272, 18694-18701; Smith, D. F. et al. Mol. Cell. Biol. 1995, 15, 6804-12), v-Src (Whitesell, L., et al. Proc. Natl. Acad. Sci. USA 1994, 91, 8324-8328) and certain transmembrane tyrosine kinases (Sepp-Lorenzino, L. et al. J. Biol. Chem. 1995, 270, 16580-16587) such as EGF receptor (EGFR) and Her2/Neu (Hartmann, F., et al. Int. J. Cancer 1997, 70, 22 1-9; Miller, P. et al. Cancer Res. 1994, 54, 2724-2730; Mimnaugh, E. G., et al. J. Biol. Chem. 1996, 271, 22796-80 1; Schnur, R. et al. J. Med. Chem. 1995, 38, 3806-3812), CDK4, and mutant p53. Erlichman et al. Proc. AACR 2001, 42, abstract 4474. The HSP90 inhibitor-induced loss of these proteins leads to the selective disruption of certain regulatory pathways and results in growth arrest at specific phases of the cell cycle (Muise-Heimericks, R. C. et al. J. Biol. Chem. 1998, 273, 29864-72), and apoptosis, and/or differentiation of cells so treated (Vasilevskaya, A. et al. Cancer Res., 1999, 59, 3935-40). HSP90 inhibitors thus hold great promise for the treatment and/or prevention of many types of cancers and proliferative disorders, and also hold promise as traditional antibiotics.

In addition to anti-cancer and antitumorigenic activity, HSP90 inhibitors have also been implicated in a wide variety of other utilities, including use as anti-inflammation agents, anti-infectious disease agents, agents for treating autoimmunity, agents for treating stroke, ischemia, multiple sclerosis, cardiac disorders, central nervous system related disorders and agents useful in promoting nerve regeneration (See, e.g, Rosen et al. WO 02/09696 (PCT/US01/23640); Degranco et al. WO 99/51223 (PCT/US99/07242); Gold, U.S. Pat. No. 6,210,974 B1; DeFranco et al., U.S. Pat. No. 6,174,875. Overlapping somewhat with the above, there are reports in the literature that fibrogenetic disorders including but not limited to scleroderma, polymyositis, systemic lupus, rheumatoid arthritis, liver cirrhosis, keloid formation, interstitial nephritis, and pulmonary fibrosis also may be treatable with HSP90 inhibitors. Strehlow, WO 02/02123 (PCT/US01/20578). Still further HSP90 modulation, modulators and uses thereof are reported in Application Nos. PCT/US03/04283, PCT/US02/35938, PCT/US02/16287, PCT/US02/06518, PCT/US98/09805, PCT/US00/09512, PCT/US01/09512, PCT/US01/23640, PCT/US01/46303, PCT/US01/46304, PCT/US02/06518, PCT/US02/29715, PCT/US02/35069, PCT/US02/35938, PCT/US02/39993, 60/293,246, 60/371,668, 60/335,391, 60/128,593, 60/337,919, 60/340,762, 60/359,484, 60/331,893 and 60/666,899.

### SUMMARY OF THE INVENTION

The present invention provides a method of treating an individual having an HSP90 mediated disorder comprising selecting a synthetic heterocyclic HSP90 inhibitor wherein the activity of the HSP90 inhibitor is substantially independent of multi drug resistance, and administering to said individual a pharmaceutical composition comprising a pharmaceutically effective amount of the selected synthetic heterocyclic HSP90 inhibitor. In other words, the activity of the selected HSP90 inhibitor is substantially independent of MRP expression (e.g., P-gp expression).

In one embodiment, the selecting step comprises determining whether said compound is a substrate of one or more MDR family members (e.g., P-gp). In one embodiment, the HSP90 inhibiting activity of said compound in cells without MDR expression (e.g., P-gp expression) is substantially the same as the inhibiting activity of said compound in cells with MDR expression (e.g., P-gp expression). The ratio of the HSP90 IC50 of said compound in cells without MDR expression (e.g., P-gp expression) and cells with MDR expression (e.g., P-gp expression) can be less than 1:10, less than 1:5, less than 1:4, less than 1:3, less than 1:2 or less than 1:1.5. In one embodiment, the ratio of the HSP90 IC50 of said compound in cells without MDR expression (e.g., P-gp expression) and with MDR expression (e.g., P-gp expression) is about 1:1.

In another embodiment, the HSP90 inhibitor is a purine analog.

In yet another embodiment, the HSP90 inhibitor is a heterocyclic compound selected from the group consisting of 2-aminopurines, pyrazolopyridines, pyrrolopyrimidines, alkynyl pyrrolopyrimidines and triazopyrimides. Preferred compounds that can be employed in conjunction with the method of the invention include compounds having the general formulae below. Specific compounds are listed in the appended claims.

A. Compounds of Formula A, tautomer, pharmaceutically acceptable salt thereof, or prodrug thereof: 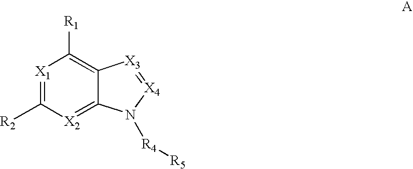
or tautomer or pharmaceutically acceptable salt or prodrug thereof, wherein

X₁ and X₂ are the same or different and each is nitrogen or —CR⁶;

X₃ is nitrogen or —CR³ wherein R³ is hydrogen, OH, a keto tautomer, —OR⁸, —CN, halogen, lower alkyl, or —C(O)R⁹;

X₄ is nitrogen or a group CR⁶ when X₃ is nitrogen, and X₄ is —CR⁶R⁷ when X₃ is —CR³;

R¹ is halogen, —OR⁸, —SR⁸, or lower alkyl;

R² is —NR⁸R¹⁰;

R⁴ is —(CH₂)ₙ— where n=0-3; and

R⁵ is alkyl, aryl, heteroaryl, alicyclic, heterocyclic, all optionally bi- or tricyclic, and optionally substituted with H, halogen, lower alkyl, lower alkenyl, lower alkynyl, lower aryl, lower alicyclic, aralkyl, aryloxyalkyl, alkoxyalkyl, perhaloalkyl, perhaloalkyloxy, perhaloacyl, —N₃, —SR⁸, —OR⁸, —CN, —CO₂R⁹, —NO₂, or —NR⁸R¹⁰;

R⁸ is hydrogen, lower alkyl, lower aryl, or —(CO)R⁹;

R⁹ is lower alkyl, lower aryl, lower heteroaryl, —NR⁸R¹⁰ or —OR¹¹;

R¹¹ is lower alkyl or lower aryl; and

R¹⁰ is hydrogen or lower alkyl.

B. Compounds of Formula II, tautomer, pharmaceutically acceptable salt thereof, or prodrug thereof:

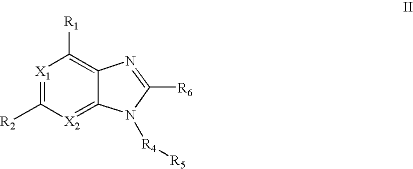

wherein

X₁ and X₂ are the same or different and each is nitrogen or —CR⁶;

R¹ is halogen, OR⁸, SR⁸, or lower alkyl;

R² is —NR⁸R¹⁰;

R⁴ is —(CH₂)ₙ— where n=0-3;

R⁶ is hydrogen, halogen, lower alkyl, —SR⁸, —OR⁸, —NR⁸R¹⁰, —N₃, —CN, or —C(O)R⁹;

R⁵ is alkyl, aromatic, heteroaromatic, alicyclic, or heterocyclic, all optionally bi- or tri-cyclic, and all optionally substituted with H, halogen, lower alkyl, —SR⁸, —OR⁸, —CN, —CO₂R⁹, —NO₂, or —NR⁸R¹⁰;

R⁸ is hydrogen, lower alkyl, lower aryl, or —(CO)R⁹;

R⁹ is lower alkyl, lower aryl, lower heteroaryl, —NR⁸R¹⁰ or —OR¹¹;

R¹¹ is lower alkyl or lower aryl; and

R¹⁰ is hydrogen or lower alkyl.

C. Compounds of Formula IV, tautomer, pharmaceutically acceptable salt thereof, or prodrug thereof:

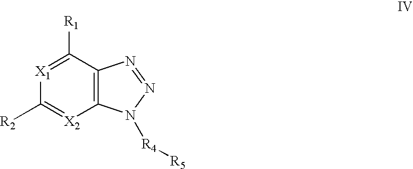

wherein

X₁ and X₂ are the same or different and each is nitrogen or CR⁶;

R¹ is halogen, —OR⁸, —SR⁸, or lower alkyl;

R² is —NR⁸R¹⁰;

R⁴ is —(CH2)ₙ,— where n=0-3;

R⁵ is alkyl, aryl, heteroaryl, alicyclic, heterocyclic, all optionally bi- or tricyclic, and all optionally substituted with H, halogen, lower alkyl, —SR⁸, —OR⁸, —CN, —CO₂R⁹, —NO₂, or —NR⁸R¹⁰;

R⁸ is hydrogen, lower alkyl, lower aryl or —(CO)R⁹;

R⁹ is lower alkyl, lower aryl, lower heteroaryl, —NR⁸R¹⁰ or —OR¹¹;

R¹¹ is lower alkyl or lower aryl; and

R¹⁰ is hydrogen or lower alkyl.

D. compounds of Formula III, tautomer, pharmaceutically acceptable salt thereof, or prodrug thereof:

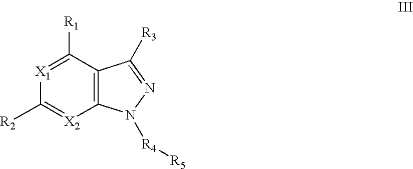

wherein

X₁ and X₂ are the same or different and each is nitrogen or CR⁶;

R₁ is halogen, —OR⁸, —SR⁸ or lower alkyl;

R² is —NR⁸R¹⁰;

R³ is hydrogen, OH or keto tautomer, —OR⁸, halogen, —CN, lower alkyl or —C(O)R⁹;

R⁴ is —(CH₂)ₙ— where n=0-3;

R⁵ is alkyl, aryl, heteroaryl, alicyclic, heterocyclic, all optionally bi- or tricyclic, and all optionally substituted with H, halogen, lower alkyl, —SR⁸, —OR⁸, —CN, —CO₂R⁹, —NO₂ or —NR⁸R¹⁰;

R⁸ is hydrogen, lower alkyl, lower aryl or —(CO)R⁹;

R⁹ is lower alkyl, lower aryl, lower heteroaryl, —NR⁸R¹⁰ or OR¹¹;

R¹¹ is lower alkyl or lower aryl; and

R¹⁰ is hydrogen or lower alkyl.

E. Compounds of Formula F, tautomer, pharmaceutically acceptable salt thereof, or prodrug thereof:

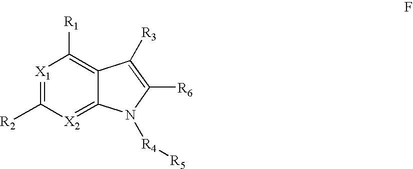

wherein

X₁ and X₂ are the same or different and each is nitrogen or —CR⁶;

R₁ is halogen, —OR⁸, —SR⁸ or lower alkyl;

R² is —NR⁸R¹⁰;

R³ is hydrogen, OH or a keto tautomer, —OR⁸, halogen, —CN, lower alkyl or —C(O)R⁹;

R⁴ is —(CH₂)ₙ— where n=0-3;

R⁶ is hydrogen, halogen, lower alkyl, —SR⁸, —OR⁸, —NR⁸R¹⁰, —N₃, or —C(O)R⁹;

R⁵ is alkyl, aromatic, heteroaromatic, alicyclic, heterocyclic, all optionally bi- or tricyclic, and all optionally substituted with H, halogen, lower alkyl, —SR⁸, —OR⁸, —CN, —CO₂R⁹, —NO₂, or —NR⁸R¹⁰;

R⁸ is hydrogen, lower alkyl, lower aryl, or —(CO)R⁹;

R⁹ is lower alkyl, lower aryl, lower heteroaryl, —NR⁸R¹⁰ or —OR¹¹;

R¹¹ is lower alkyl or lower aryl; and

R¹⁰ is hydrogen or lower alkyl;

F. Compounds of Formula IIA, tautomer, pharmaceutically acceptable salt thereof, or prodrug thereof:

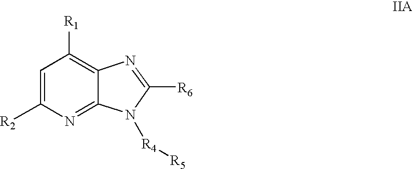

wherein

R₁ is halogen, —OR⁸, —SR⁸ or lower alkyl;

R² is —NR⁸R¹⁰;

R⁴ is —(CH₂)ₙ— where n=0-3;

R⁶ is hydrogen, halogen, lower alkyl, —SR⁸, —OR⁸, —NR⁸R¹⁰, —N₃, —CN or C(O)R⁹;

R⁵ is alkyl, aromatic, hetero aromatic, alicyclic, heterocyclic, all optionally bi- or tricyclic, and all optionally substituted with H, halogen, lower alkyl, —SR⁸, —OR⁸, —CN, —CO₂R⁹, —NO₂, or —NR⁸R¹⁰;

R⁸ is hydrogen, lower alkyl, lower aryl, or —(CO)R⁹;

R⁹ is lower alkyl, lower aryl, lower heteroaryl, NR⁸R¹⁰ or OR¹¹;

R¹¹ is lower alkyl or lower aryl; and

R¹⁰ is hydrogen or lower alkyl.

G. Compounds of Formula IIB, tautomer, pharmaceutically acceptable salt thereof, or prodrug thereof:

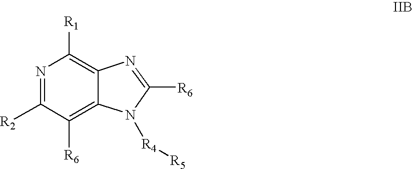

wherein

R¹ is halogen, —OR⁸, —SR⁸ or lower alkyl;

R² is —NR⁹R¹⁰;

R⁴ is —(CH₂)ₙ—, where n=0-3;

R⁶ is hydrogen, halogen, lower alkyl, —SR⁸, —OR⁸, —NR⁸R¹⁰, —N₃, —CN or —C(O)R⁹;

R⁵ is alkyl, aromatic, heteroaromatic, alicyclic, heterocyclic, all optionally bi- or tricyclic, and all optionally substituted with H, halogen, lower alkyl, —SR⁸, —OR⁸, —CN, —CO₂R⁹, —NO₂, or —NR⁸R¹⁰;

R⁸ is hydrogen, lower alkyl, lower aryl, or —(CO)R⁹;

R⁹ is lower alkyl, lower aryl, lower heteroaryl, —NR⁸R¹⁰ or —OR¹¹;

R¹¹ is lower alkyl or lower aryl; and

R¹⁰ is hydrogen or lower alkyl.

H. Compounds of Formula IA, tautomer, pharmaceutically acceptable salt thereof, or prodrug thereof: 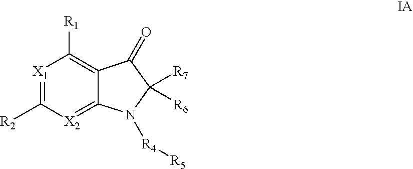
wherein:

X₁ and X₂ are the same or different and each is nitrogen or a group —CR⁶;

R¹ is halogen, —OR⁸, —SR⁸, or lower alkyl;

R² is —NR⁸R¹⁰;

R⁴ is —(CH₂)ₙ— where n=0-3;

R⁵ is alkyl, aromatic, heteroaromatic, alicyclic, heterocyclic, all optionally bi- or tricyclic, and all optionally substituted with H, halogen, lower alkyl, —SR⁸, —OR⁸, —CN, —CO₂R⁹, —NO₂, or —NR⁸R¹⁰;

R⁶ is hydrogen, halogen, lower alkyl, —SR⁸, —OR⁸, —NR⁸R¹⁰, —N₃, —CN, —C(O)R⁹, or together with R⁷ is carbonyl (C═O);

R⁷ is independently selected from hydrogen, lower alkyl or together with R⁶ is carbonyl (C═O);

R⁸ is hydrogen, lower alkyl, lower aryl, or —(CO)R⁹;

R⁹ is lower alkyl, lower aryl, lower heteroaryl, —NR⁸R¹⁰ or —OR¹¹; R¹¹ is lower alkyl or lower aryl; and

R¹⁰ is hydrogen or lower alkyl.

I. Compounds of Formula IC, tautomer, pharmaceutically acceptable salt thereof, or prodrug thereof: 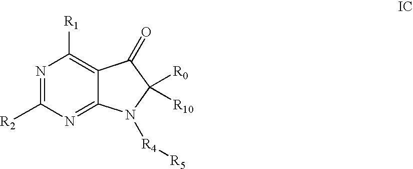
wherein:

R⁰ is hydrogen, halogen, lower alkyl, —SR⁸, —OR⁸, —CN or —NHR⁸;

R¹ is halogen, —OR¹¹, —SR¹¹ or lower alkyl;

R¹ is —NH₂;

R⁴ is —CHR¹²—;

R⁵ is aryl, heteroaryl, alicyclic, or heterocyclic, wherein:
the aryl group is substituted with 3 to 5 substituents,
the heteroaryl group is substituted with 2 to 5 substituents,
the alicyclic group is substituted with 3 to 5 substituents,
the heterocyclic group is substituted with 3 to 5 substituents, and
the substituents on R⁵ are selected from the group consisting of halogen, lower alkyl, lower alkenyl, lower alkynyl, —SR⁸, —OR⁸, —CN, —C(O)OH, —C(O)R⁹, —NO₂, —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein when R⁸ and R¹⁰ taken together optionally form a ring of 3-7 ring atoms and optionally 1-3 of the ring atoms are heteroatoms selected from the group of O, S and N;

R⁸ is hydrogen, lower alkyl, lower alkenyl, lower alkynyl, lower aryl, lower heteroaryl, or —C(O)R⁹;

R⁹ is H, lower alkyl, lower aryl, lower heteroaryl, —NR¹⁰R¹⁰, or —OR¹¹, wherein

R¹⁰ and R¹⁰ taken together optionally form a ring of 3-7 ring atoms and optionally 1-3 of the ring atoms are heteroatoms selected from the group of O, S and N;

R¹⁰ is hydrogen, lower alkyl, lower heteroaryl, lower aryl, lower alkenyl, or lower alkynyl;

R¹¹ is lower alkyl, lower alkenyl, lower alkynyl, lower heteroaryl or lower aryl;

R¹² is hydrogen or lower alkyl; and

R⁰ and R¹⁰ taken together optionally form an exocyclic double bond which is optionally substituted, or optionally form a ring of 3-7 ring atoms and optionally 1-3 of the ring atoms are heteroatoms selected from the group of O, S and N.

J. Compounds of Formula ID, tautomer, pharmaceutically acceptable salt thereof, or prodrug thereof: 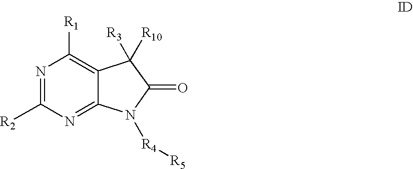
wherein:

R¹ is halogen, —OR¹¹, —SR¹¹ or lower alkyl; —

R² is —NH₂;

R³ is selected from the group consisting of hydrogen, halogen, —SR⁸, —OR⁸, —CN, —C(O)R⁹, —C(O)OH, —NO₂, —NR⁸R¹⁰, lower alkyl, lower alkenyl, lower alkynyl, lower perhaloalkyl, aryl, heteroaryl, alicyclic, heterocyclic, all optionally substituted, wherein:
the aryl, heteroaryl, alicyclic and heterocyclic groups are optionally mono-, bi- or tri-cyclic, R⁸ and R¹⁰ taken together optionally form a ring of 3-7 ring atoms and optionally 1-3 of the ring atoms are heteroatoms selected from the group of O, S and N, and
the optional substituents on R³ are selected from the group consisting of halogen, lower alkyl, lower alkenyl, lower alkynyl, —SR⁸, —OR⁸, —CN, —C(O)OH, —C(O)R⁹, —NO₂, —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;

R⁴ is —CHR¹²—;

R⁵ is aryl, heteroaryl, alicyclic, or heterocyclic, wherein
the aryl group is substituted with 3 to 5 substituents,
the heteroaryl group is substituted with 2 to 5 substituents,
the alicyclic group is substituted with 3 to 5 substituents,
the heterocyclic group is substituted with 3 to 5 substituents, and
the substituents on R⁵ are selected from the group consisting of halogen, lower alkyl, lower alkenyl, lower alkynyl, —SR⁸, —OR⁸, —CN, —C(O)OH, —C(O)R⁹, —NO₂, —NR⁹R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;

R⁸ is hydrogen, lower alkyl, lower alkenyl, lower alkynyl, lower aryl, lower heteroaryl, or —C(O)R⁹;

R⁹ is H, lower alkyl, lower aryl, lower heteroaryl, —NR¹⁰R¹⁰, or —OR¹¹, wherein when R¹⁰ and R¹⁰ taken together optionally form a ring of 3-7 ring atoms and optionally 1-3 of the ring atoms are heteroatoms selected from the group of O, S and N;

R¹⁰ is hydrogen, lower alkyl, lower heteroaryl, lower aryl, lower alkenyl, or lower alkynyl,

R¹¹ is lower alkyl, lower alkenyl, lower alkynyl, lower heteroaryl or lower aryl;

R¹² is hydrogen or lower alkyl; and

R³ and R¹⁰ taken together optionally form an exocyclic double bond which is optionally substituted, or optionally form a ring of 3-7 ring atoms and optionally 1-3 of the ring atoms are heteroatoms selected from the group of O, S and N.

K. compounds of Formula IE, tautomer, pharmaceutically acceptable salt thereof, or prodrug thereof: 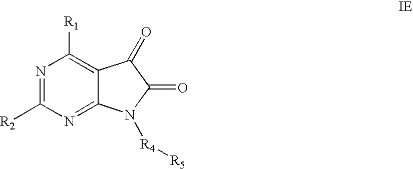
wherein:

R¹ is halogen, —OR¹¹, —SR¹¹ or lower alkyl;

R² is —NH₂;

R⁴ is —CHR¹²—;

R⁵ is aryl, heteroaryl, alicyclic, or heterocyclic, wherein
the aryl group is substituted with 3 to 5 substituents,
the heteroaryl group is substituted with 2 to 5 substituents,
the alicyclic group is substituted with 3 to 5 substituents,
the heterocyclic group is substituted with 3 to 5 substituents, and
the substituents on R⁵ are selected from the group consisting of halogen, lower alkyl, lower alkenyl, lower alkynyl, —SR⁸, —OR⁸, —CN, —C(O)OH, —C(O)R⁹, —NO₂, —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;

R⁸ is hydrogen, lower alkyl, lower alkenyl, lower alkynyl, lower aryl, lower heteroaryl, or —C(O)R⁹;

R⁹ is H, lower alkyl, lower aryl, lower heteroaryl, —NR¹⁰R¹⁰, or —OR¹¹, wherein R¹⁰ and R¹⁰ taken together optionally form a ring of 3-7 ring atoms and optionally 1-3 of the ring atoms are heteroatoms selected from the group of O, S and N;

R¹⁰ is hydrogen, lower alkyl, lower heteroaryl, lower aryl, lower alkenyl, or lower alkynyl,

R¹¹ is lower alkyl, lower alkenyl, lower alkynyl, lower heteroaryl or lower aryl; and

R¹² is hydrogen or lower alkyl.

L. Compounds of Formula IIC, tautomer, pharmaceutically acceptable salt thereof, or prodrug thereof: 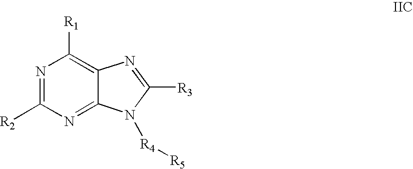
wherein:

R¹ is halogen or lower alkyl;

R² is —NR⁸R¹⁰;

R⁴ is —CHR¹²—;

R³ is hydrogen, halogen, or —CN;

R⁵ is aryl, heteroaryl, alicyclic, or heterocyclic, wherein:
the aryl group is substituted with 3 to 5 substituents,
the heteroaryl group is substituted with 2 to 5 substituents, wherein when the heteroaryl is substituted with only two substituents, the two substituents must form part of an optionally substituted fused ring,
the alicyclic group is substituted with 3 to 5 substituents,
the heterocyclic group is substituted with 3 to 5 substituents, and
the substituents are selected from the group of halogen, lower alkyl, lower alkenyl, lower alkynyl, lower aryl, lower alicyclic, arylalkyl, aryloxy, aryloxyalkyl, alkoxyalkyl, perhaloalkyl, perhaloalkyloxy, perhaloacyl, —N₃, —SR⁸, —OR⁸, —CN, —C(O)R⁹, —NO₂, —NR⁸R¹⁰, phosphonate and phosphonic acid;

R⁸ is hydrogen, lower alkyl, lower aryl, or —C(O)R⁹;

R⁹ is lower alkyl, lower aryl, lower heteroaryl, —NR¹⁰R¹⁰ or —OR¹¹;

R¹⁰ is independently hydrogen or lower alkyl;

R¹¹ is lower alkyl, lower aryl or lower heteroaryl;

R¹² is hydrogen or lower alkyl;

provided that

when R⁵ is aryl, R⁵ is not an organo-metallic cyclopentadiene;

when R⁵ is phenyl, the substituents are not 3,5 di-halo;

when R⁵ is alicyclic, the ring system does not contain any tetra-substituted sp³ ring carbons; and

when R⁵ is heterocyclic, the ring system does not contain any tetra-substituted sp³ ring carbons or the ring system is not a tetra-substituted pyrrolidine.

M. compounds of Formula IID, tautomer, pharmaceutically acceptable salt thereof, or prodrug thereof: 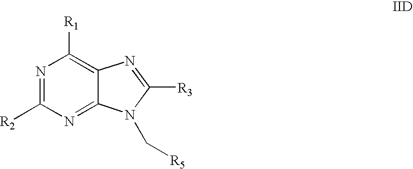
wherein:

R¹ is halogen or lower alkyl;

R² is —NR⁸R¹⁰;

R³ is hydrogen, halogen, or —CN;

R⁵ is aryl, heteroaryl, alicyclic, or heterocyclic, wherein
the aryl group is substituted with 3 to 5 substituents,
the heteroaryl group is substituted with 2 to 5 substituents, wherein when the heteroaryl is substituted with only two substituents, the two substituents must form part of an optionally substituted fused ring,
the alicyclic group is substituted with 3 to 5 substituents,
the heterocyclic group is substituted with 3 to 5 substituents, and
the substituents are selected from the group of halogen, lower alkyl, lower alkenyl, lower alkynyl, lower aryl, lower alicyclic, arylalkyl, aryloxy, aryloxyalkyl, alkoxyalkyl, perhaloalkyl, perhaloalkyloxy, perhaloacyl, —N₃, —SR⁸, —OR⁸, —CN, —C(O)R⁹, —NO₂, —NR⁸R¹⁰, phosphonate and phosphonic acid;

R⁸ is hydrogen, lower alkyl, lower aryl, or —C(O)R⁹;

R⁹ is lower alkyl, lower aryl, lower heteroaryl, —NR¹⁰R¹⁰ or —OR¹¹;

R¹⁰ is independently hydrogen or lower alkyl; and

R¹¹ is lower alkyl, lower aryl or lower heteroaryl; provided that

when R⁵ is aryl, R⁵ is not an organo-metallic cyclopentadiene;

when R⁵ is phenyl, the substituents are not 3,5 di-halo;

when R⁵ is alicyclic, the ring system does not contain any tetra-substituted sp³ ring carbons;

when R⁵ is heterocyclic, the ring system does not contain any tetra-substituted sp³ ring carbons or the ring system is not a tetra-substituted pyrrolidine.

N. compounds of Formula IIIA, tautomer, pharmaceutically acceptable salt thereof, or prodrug thereof: 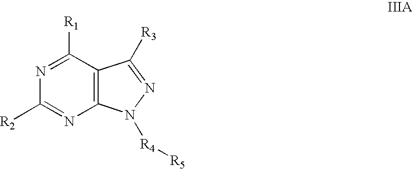
wherein:

R¹ is halogen, —OR¹¹, —SR¹¹ or lower alkyl;

R² is —NHR⁸;

R³ is selected from the group consisting of hydrogen, halogen, —SR⁸, —OR⁸, —CN, —C(O)R⁹, —CO₂H, —NO₂, —NR⁸R¹⁰, lower alkyl, lower alkenyl, lower alkynyl, lower perhaloalkyl, aryl, heteroaryl, alicyclic and heterocyclic, all optionally substituted, wherein:
the aryl, heteroaryl, alicyclic and heterocyclic groups are optionally mono-, bi- or tri-cyclic,
R⁸ and R¹⁰ taken together optionally form a ring of 3-7 ring atoms and optionally 1-2 of the ring atoms are heteroatoms selected from the group of O, S and N, and
the optional substituents on R³ are selected from the group consisting of halogen, lower alkyl, lower alkenyl, lower alkynyl, —SR⁸, —OR⁸, —CN, —C(O)R⁹, —C(O)OH, —NO₂, —NR⁸R¹⁰, lower aryl, lower heteroaryl, lower alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;

R⁴ is —CHR¹²—;

R⁵ is aryl, heteroaryl, alicyclic, or heterocyclic, wherein
the aryl group is substituted with 3 to 5 substituents,
the heteroaryl group is substituted with 2 to 5 substituents,
the alicyclic group is substituted with 3 to 5 substituents,
the heterocyclic group is substituted with 3 to 5 substituents, and
the substituents on R⁵ are selected from the group consisting of halogen, lower alkyl, lower alkenyl, lower alkynyl, —SR⁸, —OR⁸, —CN, —C(O)OH, —C(O)R⁹, —NO₂ and —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;

R⁸ is hydrogen, lower alkyl, lower alkenyl, lower alkynyl, lower aryl, lower heteroaryl or —C(O)R⁹;

R⁹ is hydrogen, lower alkyl, lower alkenyl, lower alkynyl, lower aryl, lower heteroaryl, —NR¹⁰R¹⁰ or —OR¹¹, R¹⁰ and R¹⁰ taken together optionally form a ring of 3-7 ring atoms and optionally 1-3 of the ring atoms are heteroatoms selected from the group of O, S and N;

R¹⁰ is hydrogen, lower alkyl, lower alkenyl, lower alkynyl, lower aryl or lower heteroaryl;

R¹¹ is lower alkyl, lower alkenyl, lower alkynyl, lower aryl or lower heteroaryl; and

R¹² is hydrogen or lower alkyl.

O. Compounds of Formula IIIB, tautomer, pharmaceutically acceptable salt thereof, or prodrug thereof: 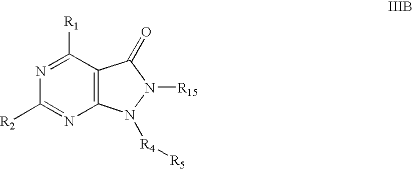
wherein:

R¹ is halogen, —OR¹¹, —SR¹¹ or lower alkyl;

R² is —NHR⁸;

R⁴ is —CHR¹²—;

R⁵ is aryl, heteroaryl, alicyclic, or heterocyclic, wherein
the aryl group is substituted with 3 to 5 substituents,
the heteroaryl group is substituted with 2 to 5 substituents,
the alicyclic group is substituted with 3 to 5 substituents,
the heterocyclic group is substituted with 3 to 5 substituents, and
the substituents on R⁵ are selected from the group consisting of halogen, lower alkyl, lower alkenyl, lower alkynyl, —SR⁸, —OR⁸, —CN, —C(O)OH, —C(O)R⁹, —NO₂ and —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;

R⁸ is hydrogen, lower alkyl, lower alkenyl, lower alkynyl, lower aryl, lower heteroaryl or —C(O)R⁹;

R⁹ is hydrogen, lower alkyl, lower alkenyl, lower alkynyl, lower aryl, lower heteroaryl, —NR¹⁰R¹⁰ or —OR¹¹, R¹⁰ and R¹⁰ taken together optionally form a ring of 3-7 ring atoms and optionally 1-3 of the ring atoms are heteroatoms selected from the group of O, S and N;

R¹⁰ is hydrogen, lower alkyl, lower alkenyl, lower alkynyl, lower aryl or lower heteroaryl;

R¹¹ is lower alkyl, lower alkenyl, lower alkynyl, lower aryl or lower heteroaryl;

R¹² is hydrogen or lower alkyl; and

R¹⁵ is hydrogen, lower alkyl, lower alkenyl or lower alknyl.

P. compounds of Formula IVA, tautomer, pharmaceutically acceptable salt thereof, or prodrug thereof: 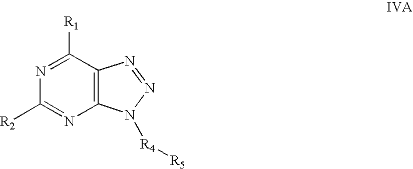
wherein:

R¹ is halogen, —OR¹¹, —SR¹¹ or lower alkyl;

R² is —NHR⁸;

R⁴ is —CHR¹²—;

R⁵ is aryl, heteroaryl, alicyclic, or heterocyclic, wherein:
the aryl group is substituted with 3 to 5 substituents,
the heteroaryl group is substituted with 2 to 5 substituents,
the alicyclic group is substituted with 3 to 5 substituents,
the heterocyclic group is substituted with 3 to 5 substituents, and
the substituents are selected from the group consisting of halogen, lower alkyl, lower alkenyl, lower alkynyl, —SR⁸, —OR⁸, —CN, —C(O)OH, —C(O)R⁹, —NO₂ and —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazol, phosphonates, phosphates, phosphoramides, sulfonates, sulfones, sulfates, sulphonamides, carbamates, ureas, thioureas, thioamides, wherein R⁸ and R¹⁰ taken together optionally form a ring of 3-7 ring atoms and optionally 1-3 of the ring atoms are heteroatoms selected from the group of O, S and N;

R⁸ is hydrogen, lower alkyl, lower alkenyl, or lower alkynyl, lower aryl, lower heteroaryl, or —C(O)R⁹;

R⁹ is H, lower alkyl, lower alkenyl, or lower alkynyl, lower aryl, lower heteroaryl, —NR¹⁰R¹⁰, or —OR¹¹, wherein R¹⁰ and R¹⁰ taken together optionally form a ring of 3-7 ring atoms and optionally 1-3 of the ring atoms are heteroatoms selected from the group of O, S and N;

R¹⁰ is hydrogen, lower alkyl, lower heteroaryl, lower aryl, lower alkenyl, or lower alkynyl,

R¹¹ is lower alkyl, lower alkenyl, lower alkynyl, lower heteroaryl or lower aryl;

and R⁴ is —CHR¹²—, —C(O)—, —C(S)—, —S(O)— or —SO₂—; and

R¹² is hydrogen or lower alkyl;

provided that when R⁵ is alicyclic, the ring system does not contain any tetra-substituted sp³ ring carbons.

Q. Compounds of Formula VI, tautomer, pharmaceutically acceptable salt thereof, or prodrug thereof: 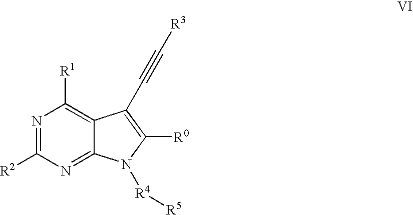
wherein:

R⁰ is selected from the group consisting of hydrogen, halogen, lower alkyl, —CN, —SR⁸, —OR⁸, and —NHR⁸;

R¹ is selected from the group consisting of halogen, —OR¹¹, —SR¹¹ and lower alkyl;

R² is —NHR⁸;

R³ is selected from the group consisting of hydrogen, —CN, —C(O)OH, —OR¹¹, —SR¹¹, —C(O)R⁹, —NR⁸R¹⁰, lower alkyl, lower alkenyl, lower alkynyl, lower perhaloalkyl, lower alkylsilyl, aryl, heteroaryl, alicyclyl and heterocyclyl, all optionally substituted, wherein:
the aryl, heteroaryl, alicyclyl and heterocyclyl groups are mono-, bi- or tri-cyclic;
R⁸ and R¹⁰ taken together with the N atom to which they are attached optionally form an optionally substituted ring comprising 3-7 ring atoms, wherein, in addition to said N atom, 0-3 of the ring atoms are heteroatoms selected from the group consisting of O, S and N;
the optional substituents on R³ are selected from the group consisting of lower alkyl, lower alkenyl, lower alkynyl, —CN, —C(O)OH, —NO₂, —SR⁸, —OR⁸, —C(O)R⁹, —NR⁸R⁸, lower aryl, heteroaryl, alicyclyl, lower heterocyclyl, arylalkyl, heteroarylalkyl, amino, alkylamino, dialkylamino, arylalkylamino, diarylamino, heteroarylamino, diheteroarylamino, arylheteroarylamino, oxo, perhaloalkyl, perhaloalkoxy, perhaloacyl, guanidinyl, pyridinyl, thiophenyl, furanyl, indolyl, indazolyl, phosphonyl, phosphatidyl, phosphoramidyl, sulfanyl, sulfinyl, sulfonyl, sulphonamidyl, carbamyl, uryl, thiouryl and thioamidyl, wherein
   R⁸ and R⁸ taken together with the N atom to which they are attached optionally form an optionally substituted ring comprising 3-7 ring atoms, wherein, in addition to said N atom, 0-3 of the ring atoms are heteroatoms selected from the group consisting of O, S and N;

R⁴ is selected from the group consisting of optionally substituted lower alkylene, —C(R²)₂—, —C(O)—, —C(S)—, —S(O)— and —SO₂—;

R⁵ is selected from the group consisting of aryl, heteroaryl, alicyclyl and heterocyclyl, wherein:
the aryl group is substituted with 2 to 5 substituents;
the heteroaryl group is substituted with 2 to 5 substituents;
the alicyclyl group is substituted with 3 to 5 substituents;
the heterocyclyl group is substituted with 3 to 5 substituents;
the substituents on R⁵ are selected from the group consisting of halogen, lower alkyl, lower alkenyl, lower alkynyl, —CN, —C(O)OH, —NO₂, —SR⁸, —OR⁸, —C(O)R⁹, —NR⁸R¹⁰, lower aryl, lower heteroaryl, lower alicyclyl, lower heterocyclyl, arylalkyl, heteroarylalkyl, thioalkyl, amino, alkylamino, dialkylamino, arylalkylamino, oxo, perhaloalkyl, perhaloalkoxy, perhaloacyl, guanidinyl, pyridinyl, thiophenyl, furanyl, indolyl, indazolyl, phosphonyl, phosphatidyl, phosphoramidyl, sulfanyl, sulfinyl, sulfonyl, sulphonamidyl, carbamyl, uryl, thiouryl and thioamidyl, wherein
R⁸ and R¹⁰ taken together with the N atom to which they are attached optionally form an optionally substituted ring comprising 3-7 ring atoms, wherein, in addition to said N atom, 0-3 of the ring atoms are heteroatoms selected from the group consisting of O, S and N;

R⁸ is selected from the group consisting of hydrogen, lower alkyl, lower alkenyl, lower alkynyl, lower heteroalkyl, lower heteroalkenyl, lower heteroalkynyl, lower aryl, lower heteroaryl and —C(O)R⁹;

R⁹ is selected from the group consisting of H, lower alkyl, lower alkenyl, lower alkynyl, lower aryl, lower heteroaryl, —NR¹⁰R¹⁰ and —OR¹¹, wherein
R¹⁰ and R¹⁰ taken together with the N atom to which they are attached optionally form an optionally substituted ring comprising 3-7 ring atoms, wherein, in addition to said N atom, 0-3 of the ring atoms are heteroatoms selected from the group consisting of O, S and N;

R¹⁰ is selected from the group consisting of hydrogen, lower alkyl, lower alkenyl, lower alkynyl, lower heteroalkyl, lower heteroalkenyl, lower heteroalkynyl, lower aryl, lower heteroaryl and —C(O)R¹¹;

R¹¹ is selected from the group consisting of lower alkyl, lower alkenyl, lower alkynyl, lower aryl and lower heteroaryl; and

R¹² is selected from the group consisting of hydrogen and lower alkyl.

R. Compounds of Formula I, or a polymorph, solvate, ester, tautomer, enantiomer, pharmaceutically acceptable salt or prodrug thereof: 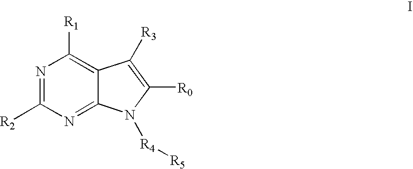
wherein:

R⁰ is selected from hydrogen, halogen, lower alkyl, —SR⁸, —OR⁸, —CN, and —NHR⁸,

R¹ is halogen, —OR¹¹, —SR¹¹ or lower alkyl;

R² is —NHR⁸;

R³ is selected from the group consisting of hydrogen, halogen, —SR⁸, —OR⁸, —CN, —C(O)R⁹, —C(O)OH, —NO₂, —NR⁸R¹⁰, lower alkyl, lower alkenyl, lower alkynyl, lower perhaloalkyl, aryl, heteroaryl, alicyclic and heterocyclic, all optionally substituted, wherein:
the aryl, heteroaryl, alicyclic and heterocyclic groups are optionally mono-, bi- or tri-cyclic,
R⁸ and R¹⁰ taken together optionally form a ring of 3-7 ring atoms and optionally 1-3 of the ring atoms are heteroatoms selected from the group of O, S and N, and
the optional substituents on R³ are selected from the group consisting of halogen, lower alkyl, lower alkenyl, lower alkynyl, —SR⁸, —OR⁸, —CN, —C(O)OH, —C(O)R⁹, —NO₂, —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;

R⁰ or R³ is —OH or —SH, the compound may exist as the corresponding (thio)keto tautomer or a mixture of keto-enol tautomers;

R⁴ is —CHR¹²—;

R⁵ is aryl, heteroaryl, alicyclic, or heterocyclic, wherein
the aryl group is substituted with 3 to 5 substituents,
the heteroaryl group is substituted with 2 to 5 substituents,
the alicyclic group is substituted with 3 to 5 substituents,
the heterocyclic group is substituted with 3 to 5 substituents, and
the substituents are selected from the group consisting of halogen, lower alkyl, lower alkenyl, lower alkynyl, —SR⁸, —OR⁸, —CN, —C(O)OH, —C(O)R⁹, —NO₂, —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;

R⁸ is hydrogen, lower alkyl, lower alkenyl, or lower alkynyl, lower aryl, lower heteroaryl, or —C(O)R⁹;

R⁹ is H, lower alkyl, lower alkenyl, lower alkynyl, lower aryl, lower heteroaryl, —NR¹⁰R¹⁰, or —OR¹¹, wherein R¹⁰ and R¹⁰ taken together optionally form a ring of 3-7 ring atoms and optionally 1-3 of the ring atoms are heteroatoms selected from the group of O, S and N;

R¹⁰ is hydrogen, lower alkyl, lower alkenyl, lower alkynyl, lower aryl or lower heteroaryl;

R¹¹ is lower alkyl, lower alkenyl, lower alkynyl, lower heteroaryl or lower aryl; and

R¹² is hydrogen or lower alkyl.

In a further embodiment, the invention provides a method, wherein the HSP90 mediated disorder is selected from the group of inflammatory diseases, infections, autoimmune disorders, stroke, ischemia, cardiac disorders, neurological disorders, fibrogenic disorders, proliferative disorders, tumors, leukemias, neoplasms, cancers, carcinomas, metabolic diseases, and malignant disease.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows how overexpression of MDR is associated with resistance to natural but not synthetic Hsp90 inhibitors. Cells were plated in 96-well plate at 10-20% confluence and treated with serial diluted compound, natural Hsp90 inhibitor, represented by 17-AAG or synthetic Hsp90 inhibitor, represented by compound VII, from 10 mM to 1 nM. 5 days later, the surviving fraction was estimated by MTS assay.

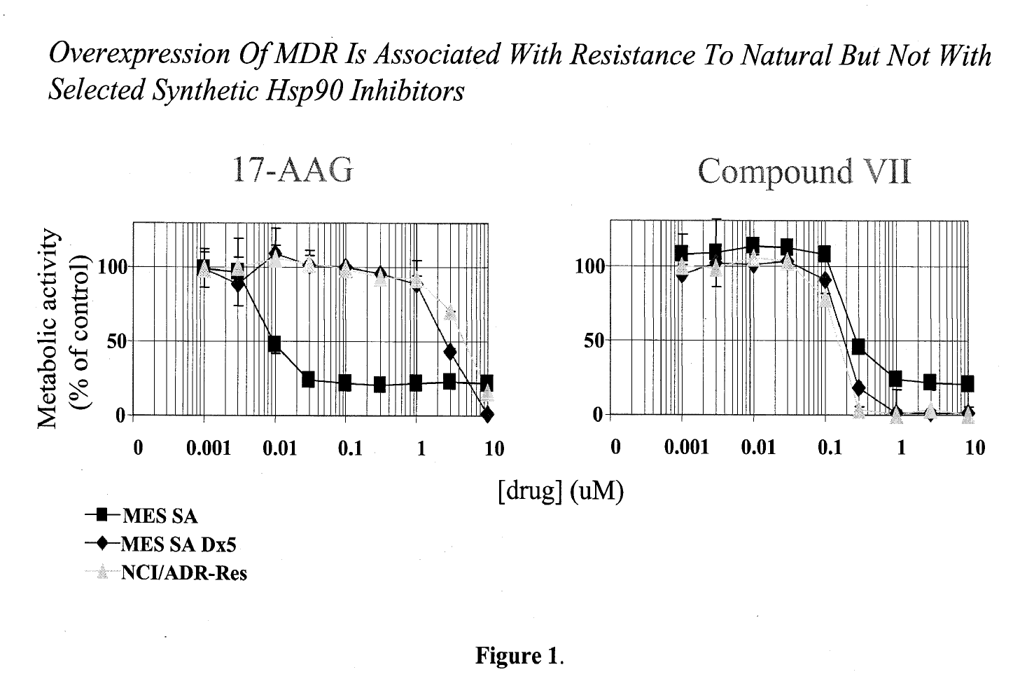

FIG. 2 shows a comparison of natural and synthetic HSP90 inhibitors in P-gp-overexpressing cells. The cell lines were plated in 96-well plate at 10-20% confluence and treated with increasing concentrations of indicated drug from 1 nM to 10 mM. 5 days later, the surviving fraction was estimated by MTS assay. IC50: concentration of drug required to inhibit cell growth by 50%. Note: Natural, but not synthetic, Hsp90 inhibitors showed dramatic increase of IC50 in P-gp over-expressing MES SA Dx5 and NCI/ADR-Res cells.

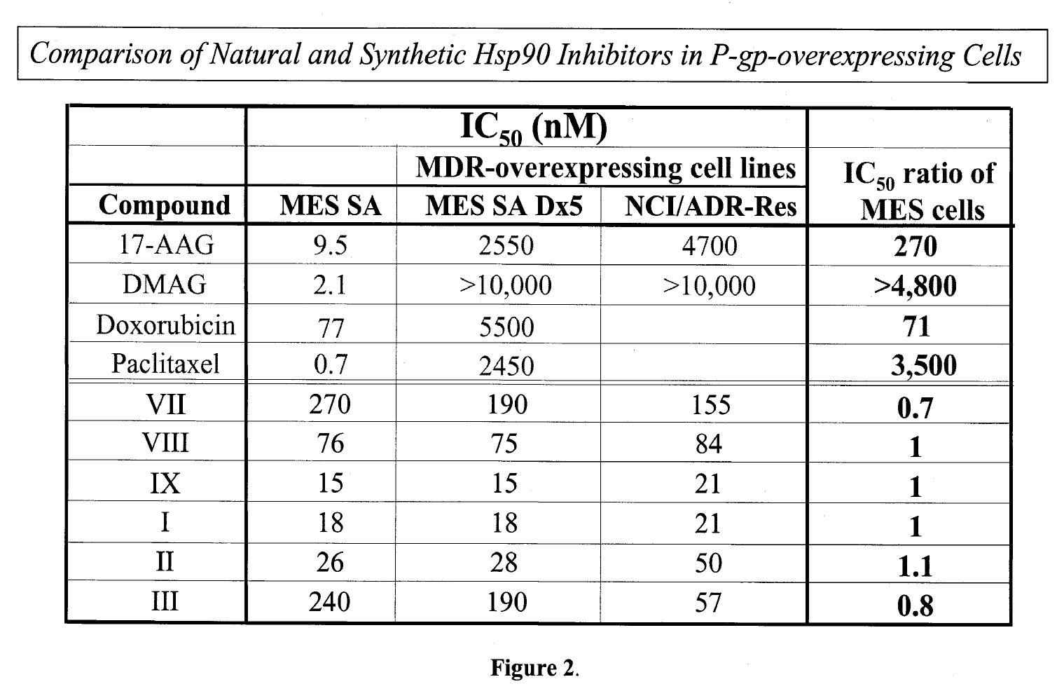

FIG. 3 shows a correlation of p-gp expression and cytotoxicity of 17-AAG in a variety of cancer cell lines. 5×10⁵ cells of each of the indicated cell lines in the graph were collected and incubated with anti-P-gp antibody (MRK16). Cell surface P-glycoprotein levels were detected with FITC-labeled anti-IgG antibody and quantified by flow cytometry. The IC50 of 17-AAG showed a linear proportion with the level of P-gp in the cell lines.

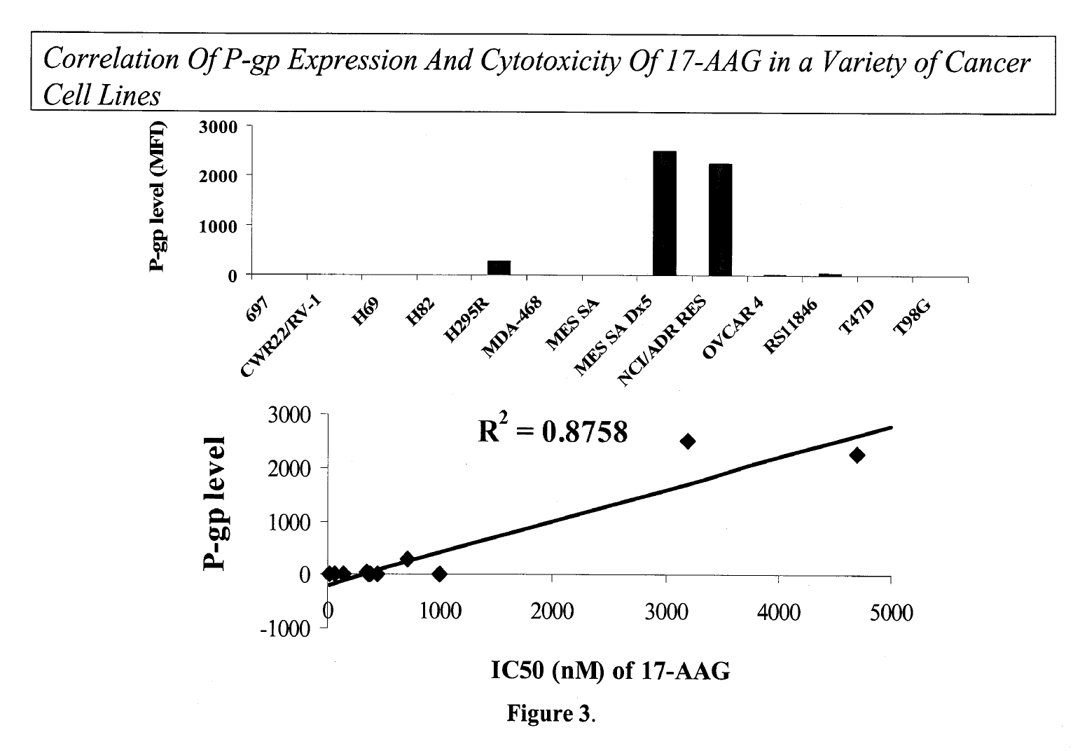

FIG. 4 shows how synthetic HSP90 Inhibitors are more potent than 17-AAG and 17-DMAG in adrenal cortical carcinoma. H295R cells were plated in 96-well plate at low confluence and were analyzed for IC50 as described in FIG. 2 with indicated compounds. Note: DMAG is also weak against adrenal cortical carcinoma, suggesting a role for MDR. To look at the effect on client degradation, H295R cells were plated in T75 flask and were treated on the next day with DMSO, 17-AAG or compound VII for 24 hours at indicated concentrations. Cells were collected and lysed. Equal amounts of protein were loaded on SDS gels. Expression of HSP90 client proteins was measured by Western blot, using specific antibodies against each protein. PI-3Kp85 (a non-client protein) was used as a loading control.

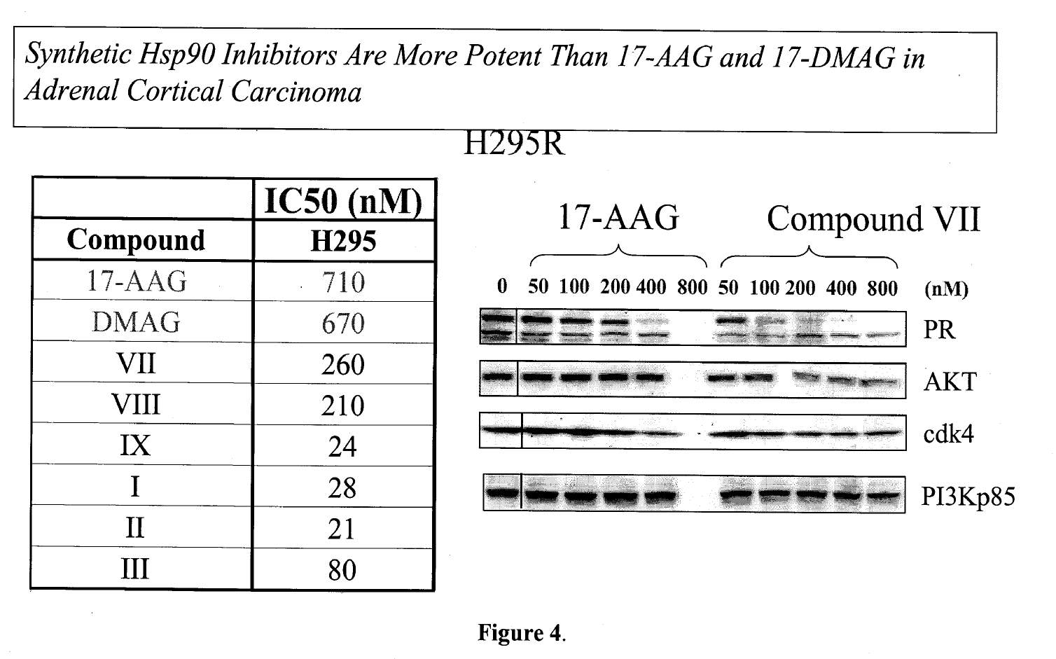

FIG. 5 shows a comparison of synthetic Hsp90 inhibitors in non P-gp and P-gp expressing cells. Some synthetic inhibitors are substrates of P-gp while others are not substrates of P-gp.

FIG. 6 shows how 17-AAG did not inhibit P-gp activity in MES Dx5 cells. 1.0×10⁶ MES SA Dx5 cells were collected and resuspended in 1 ml of serum-free DMEM medium and incubated with rhodamine 123 (a P-gp substrate) and compounds at indicated concentration for 1 h at 37° C. The cells were washed and resuspended again in 1 ml serum-free medium to incubate at 37° C. for another 2 h. Rhodamine 123 level was measured by flow cytometry using a FACScan by measuring events in the FL1 channel. Inhibition of P-gp was determined by the amount of Rhodamine 123 that was retained in the cells.

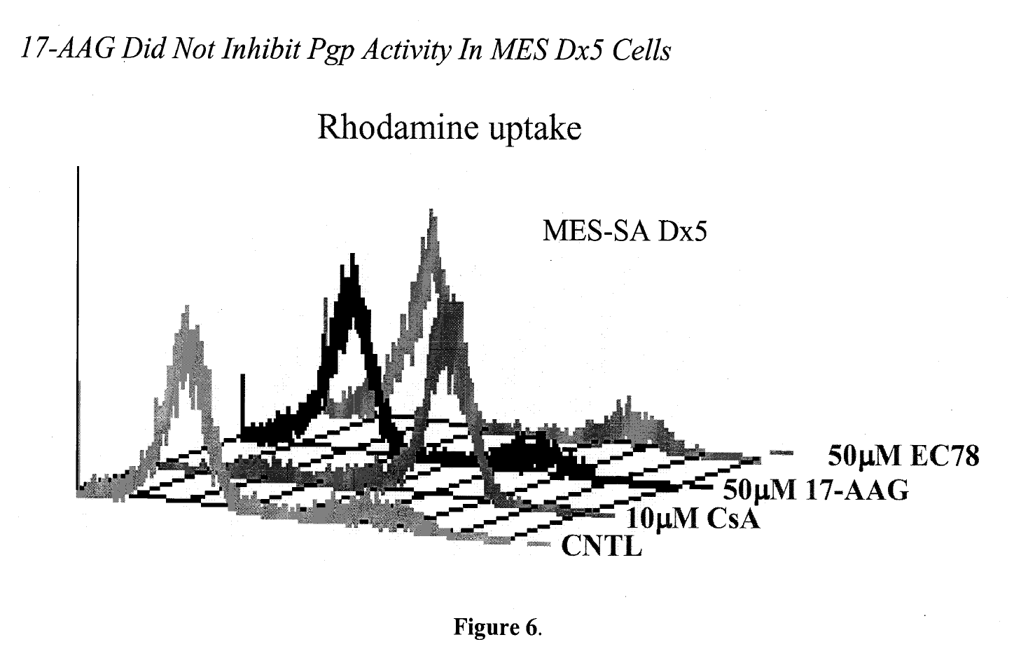

FIG. 7 shows how 17-AAG did not inhibit MDR activity in H295R cells. 1.0×10⁶ H295R adrenal cortical carcinoma cells were collected and incubated with rhodamine 123 and increasing concentration of 17-AAG or CsA (positive control). Rhodamine 123 level within cells was measured by flow cytometry as described in FIG. 9. No inhibitory effect on P-gp efflux activity was observed with 17-AAG at concentrations up to 10 mM, although CsA showed activity start from 1 mM. This is not due to the impermeability of 17-AAG, as it is more potent than CsA in H295R in cytotoxic studies.

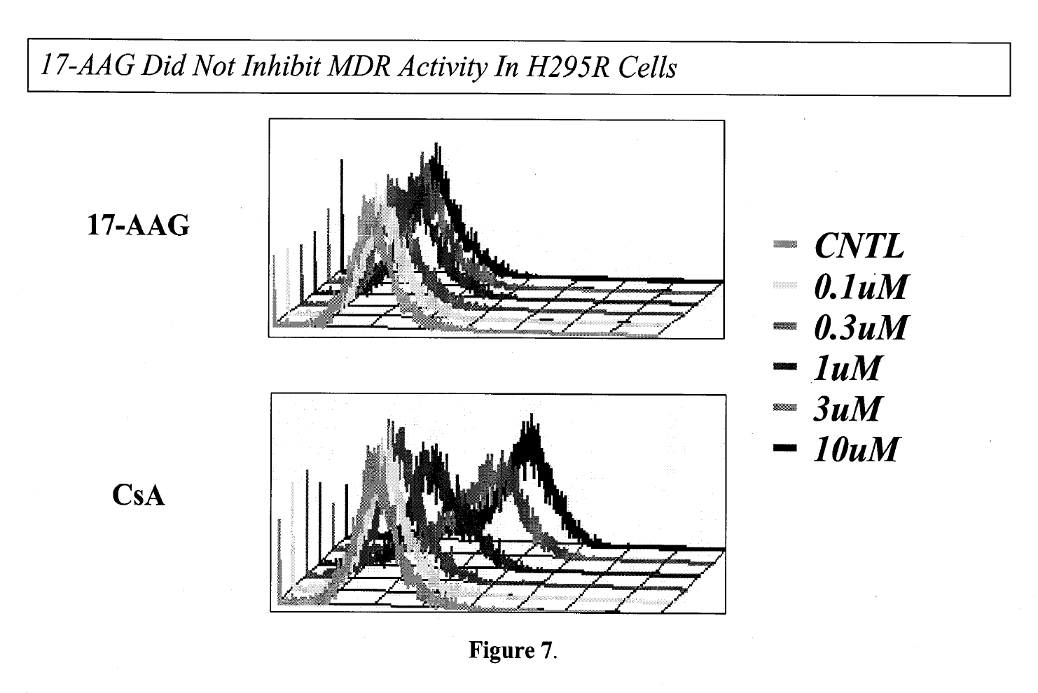

### DETAILED DESCRIPTION OF THE INVENTION

The first-in-class HSP-90 inhibitor 17-AAG has been tested in hundreds of patients, primarily in all-corners solid tumor Phase I trials, with sporadic evidence of clinical activity. A major contributor to drug resistance commonly observed in heavily-pretreated Phase I patients is the upregulation of drug efflux pumps, especially the multidrug resistance protein P-glycoprotein (P-gp). Here we report that 17-AAG and other ansamycin drugs are highly sensitive P-gp substrates, but that a new generation of potent synthetic Hsp90 inhibitors are largely independent of MDR. The activity of a panel of ansamycin and synthetic compounds was tested in a variety of cell lines expressing P-gp or MRP (MDR-related protein) at various levels. Overexpression of P-glycoprotein markedly increased the IC₅₀s of 17-AAG and 17-DMAG in client protein degradation, biomarker secretion and cytotoxicity assays (≧500 fold). Increased IC50 values are indicative of a decrease in or loss of activity. An inverse correlation between P-gp level and the cytotoxicity of 17-AAG was observed. The phenomena could be reversed by co-administration of P-gp or MRP inhibitors, which significantly increased the potency of 17-AAG. By contrast, a group of synthetic heterocyclic HSP90 inhibitor were not affected by either P-gp or MRP expression. Furthermore, the synthetic Hsp90 inhibitors were considerably more active against adrenocortical carcinoma, a tumor that naturally expresses P-gp. Interestingly, although 17-AAG was shown to be a preferred substrate for P-gp, unlike many other P-gp substrates, it exhibited no inhibitory activity against the protein. Our results indicate that the activity of 17-AAG and other ansamycins may be curtailed in tumors expressing P-gp but that this property is not inherent in all Hsp90 inhibitors. Thus, this group of synthetic heterocyclic Hsp90 inhibitors may have broader application against tumors with acquired multidrug resistance and in cancers located in organs protected by P-gp, such as the adrenal glands, brain, heart, liver, kidneys, uterus and testis.

Hsp90 is an attractive target in cancer therapy due to its critical role in mediating the maturation and stability of a variety of cancer associated proteins. By far, several Hsp90 inhibitors are under clinical investigation, with 17-AAG in the lead. Many of the clinical candidates, including KOS-1022 (DMAG), KOS-953, CNF1010 and IPI-504, are also semi-synthetic geldanamycin derivatives and are structurally related to 17-AAG. Clinical trial of 17-AAG in over 400 patients, primarily in all-corners solid tumor, had shown sporadic evidence of clinical activity. A major contributor to drug resistance commonly observed in heavily-pretreated Phase I patients is the upregulation of drug efflux pumps, especially the multidrug resistance protein P-glycoprotein (P-gp). These raised the possibility that increased P-gp level in these patients may have an effect on the response of the patients to 17-AAG and its analogues.

Multidrug resistance (MDR) is a significant obstacle in cancer therapy. Expression of P-gp, encoded by MDR gene, confers resistance to a broad range of structurally and functionally unrelated chemotherapeutic agents and certain mechanism-based drugs, including Gleevec. This protein functions as drug efflux pump at cellular membrane and causes increased efflux of anticancer drugs. It not only elicits drug resistance at cellular levels, but was also identified to alter the pharmacokinetics of various drugs and associated with poor bioavailability. P-gp is highly expressed in colon, kidney, adrenocortical and hepatocellular cancers and intermediately expressed in breast, lung, neuroblastoma cancers, etc. It was also shown that constant exposure to drugs which are P-gp substrates induces the expression of the multidrug resistant protein and results in acquired resistance of the tumor.

Previous evidence has shown that the IC50 of 17-AAG was elevated significantly in HCT15 due to the MDR phenotype of the cell line. To investigate whether 17-AAG and its analogs are P-gp substrates, we performed cytotoxicity assay in a variety of cell lines expressing P-gp or MDR related protein (MRP) to various extend. Our results have shown that 17-AAG, 17-DMAG are both P-gp substrates, whose IC50s were dramatically elevated in P-gp over-expressing cell lines. The phenomena was reversed by co-administration of P-gp or MRP inhibitors, which significantly increased the potency of 17-AAG. Interestingly, although 17-AAG was shown to be a preferred substrate for P-gp, unlike many other P-gp substrates, it exhibited no inhibitory activity against the protein. By contrast, selected compounds from a new generation of potent synthetic Hsp90 inhibitors are not affected by P-gp expression. Other synthetic compounds, however, have shown activity that is dependent on P-gp expression, for example compound XI whose IC50 was moderately (10-20 fold) increased in P-gp+++ lines. Furthermore, selected synthetic HSP-90 inhibitors were considerably more active against adrenocortical carcinoma, a tumor that naturally expresses P-gp. These synthetic molecules have entirely novel chemical entity, with good anti-tumor activity and bioavailability, and are orally available. Our results indicated that this new generation of HSP-90 inhibitors may have broader application against tumors with acquired multidrug resistance and in cancers associated with the immune system or located in organs protected by P-gp, such as the adrenal glands, brain, heart, liver, kidneys, uterus and testis.

A variety of in vitro and in vivo assays are available to test the effect of the compounds of the invention on HSP-90. HSP-90 competitive binding assays and functional assays can be performed as known in the art by substituting in the compounds of the invention. Chiosis et al. Chemistry & Biology 2001, 8, 289-299, describe some of the known ways in which this can be done. For example, competition binding assays using, e.g., geldanamycin or 17-AAG as a competitive binding inhibitor of HSP-90 can be used to determine relative HSP-90 affinity of the compounds of the invention by immobilizing the compound of interest or other competitive inhibitor on a gel or solid matrix, preincubating HSP-90 with the other inhibitor, passing the preincubated mix over the gel or matrix, and then measuring the amount of HSP90 that retains or does not retain on the gel or matrix.

Downstream effects can also be evaluated based on the known effect of HSP-90 inhibition on function and stability of various steroid receptors and signaling proteins including, e.g., Raf1 and Her2. Compounds of the present invention induce dose-dependent degradation of these molecules, which can be measured using standard techniques. Inhibition of HSP-90 also results in up-regulation of HSP-90 and related chaperone proteins that can similarly be measured. Antiproliferative activity on various cancer cell lines can also be measured, as can morphological and functional differentiation related to HSP-90 inhibition.

Many different types of methods are known in the art for determining protein concentrations and measuring or predicting the level of proteins within cells and in fluid samples. Indirect techniques include nucleic acid hybridization and amplification using, e.g., polymerase chain reaction (PCR). These techniques are known to the person of skill and are discussed, e.g., in Sambrook, Fritsch & Maniatis Molecular Cloning: A Laboratory Manual, 2nd ed., Cold Spring Harbor Laboratory, Cold Spring Harbor, N.Y., 1989; Ausubel, et al. Current Protocols in Molecular Biology, John Wiley & Sons, NY, 1994, and, as specifically applied to the quantification, detection, and relative activity of HER2/Neu in patient samples, e.g., in U.S. Pat. Nos. 4,699,877, 4,918,162, 4,968,603, and 5,846,749. A brief discussion of two generic techniques that can be used follows.

The determination of whether cells overexpress or contain elevated levels of Her2 can be determined using well known antibody techniques such as immunoblotting, radioimmunoassays, western blotting, immunoprecipitation, enzyme-linked immunosorbant assays (ELISA), and derivative techniques that make use of antibodies directed against Her2. As an example, Her2 expression in breast cancer cells can be determined with the use of an immunohistochemical assay, such as the Dako Hercep™ test (Dako Corp., Carpinteria, Calif.). The Hercep™ test is an antibody staining assay designed to detect Her2 overexpression in tumor tissue specimens. This particular assay grades Her2 expression into four levels: 0, 1, 2, and 3, with level 3 representing the highest level of Her2 expression. Accurate quantitation can be enhanced by employing an Automated Cellular Imaging System (ACTS) as described, e.g., by Press, M. et al. Modern Pathology 2000, 13, 225A.

Antibodies, polyclonal or monoclonal, can be purchased from a variety of commercial suppliers, or may be manufactured using well-known methods, e.g., as described in Harlow et al. Antibodies: A Laboratory Manual, 2nd ed; Cold Spring Harbor Laboratory, Cold Spring Harbor, N.Y., 1988.

Her2 overexpression can also be determined at the nucleic acid level since there is a reported high correlation between overexpression of the Her2 protein and amplification of the gene that codes for it. One way to test this is by using RT-PCR. The genomic and eDNA sequences for Her2 are known. Specific DNA primers can be generated using standard, well-known techniques, and can then be used to amplify template already present in the cell. An example of this is described in Kurokawa, H. et al. Cancer Res. 2000, 60, 5887-5894. PCR can be standardized such that quantitative differences are observed as between normal and abnormal cells, e.g., cancerous and noncancerous cells. Well known methods employing, e.g., densitometry, can be used to quantitate and/or compare nucleic acid levels amplified using PCR.

Similarly, fluorescent in situ hybridization (FISH) assays and other assays can be used, e.g., Northern and/or Southern blotting. These rely on nucleic acid hybridization between the Her2 gene or mRNA and a corresponding nucleic acid probe that can be designed in the same or a similar way as for PCR primers, above. See, e.g., Mitchell M S, and Press M. F. Oncol., Suppl. 1999, 12, 108-116. For FISH, this nucleic acid probe can be conjugated to a fluorescent molecule, e.g., fluorescein and/or rhodamine, that preferably does not interfere with hybridization, and which fluorescence can later be measured following hybridization. See, e.g., Kurokawa, H et al, Cancer Res. 2000, 60, 5887-5894 (describing a specific nucleic acid probe having sequence 5′-FAM-NucleicAcid-TAMRA-p-3′ sequence). ACIS-based approaches as described above can be employed to make the assay more quantitative (de la Torre-Bueno, J., et al. Modern Pathology 2000, 13, 221A).

Immuno and nucleic acid detection can also be directed against proteins other than HSP90 and HER2, which proteins are nevertheless affected in response to HSP90 inhibition.

The following examples are offered by way of illustration only and are not intended to be limiting of the full scope and spirit of the invention.

### Exemplary Compounds

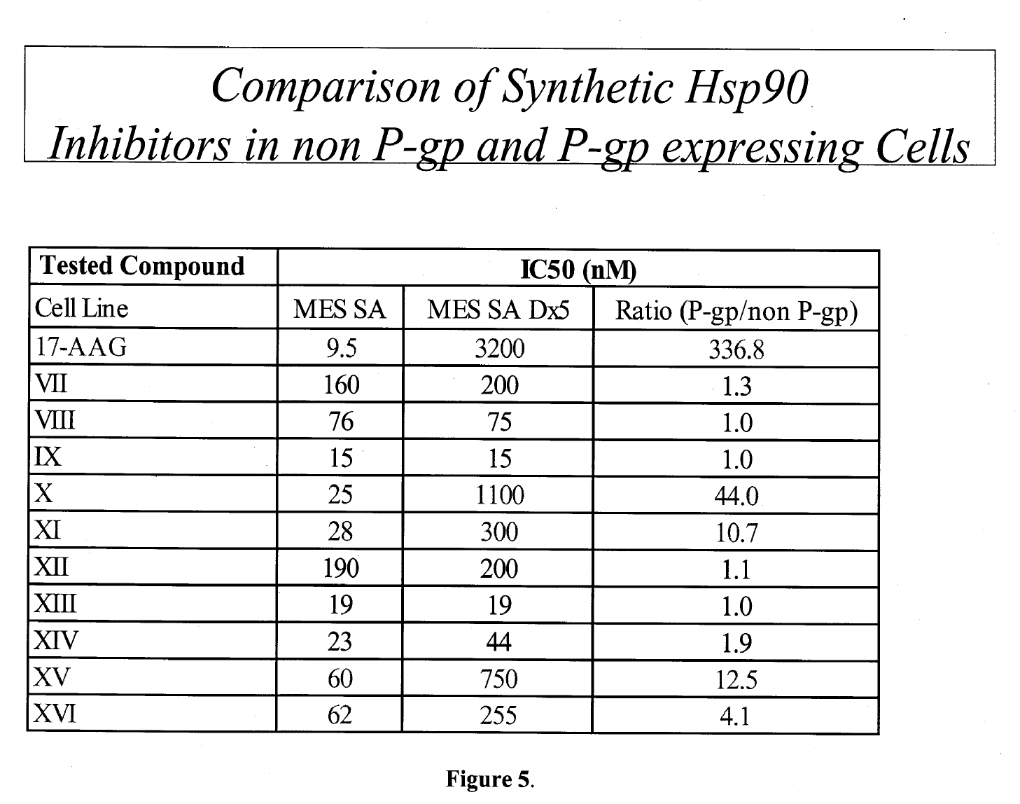

As shown in FIG. 5, some of the synthetic compounds below are not substrates of P-gp and are MDR independent while other synthetic compounds listed below are substrates of P-gp. HSP-90 inhibiting activity of synthetic compounds that are P-gp substrates would be affected in P-gp expressing cells. Assays for determining whether a compound is a substrate of P-gp or other cell protecting protein are well known to those of ordinary skill in the art. Examples of assays that can employed in conjunction with the present invention are described in the following references: “CpG hypermythylation of MDR1 Gene Contribute to the Pathogenesis and Progression of Human Prostate Cancer” Enokida et al. Cancer research 64, 5956-62 (September 2004) and “Allosteric Modulation of Human P-glycoprotein” Maki et al. The Journal of Biological Chemistry Vol. 278, No. 20 18132-39 (May 2003). Employing such assays in identifying synthetic HSP-90 inhibitors that are MDR independent is with the scope of the present invention.
Compound I: 5-(2-amino-4-chloro-7-((4-methoxy-3,5-dimethylpyridin-2-yl)methyl)-7H-pyrrolo[2,3-d]pyrimidin-5-yl)pent-4-yn-1-ol
Compound II: 4-chloro-7-((4-methoxy-3,5-dimethylpyridin-2-yl)methyl)-5-(pyridin-2-ylethynyl)-7H-pyrrolo[2,3-d]pyrimidin-2-amine
Compound III: 5-(5-aminopent-1-ynyl)-4-chloro-7-((4-methoxy-3,5-dimethylpyridin-2-yl)methyl)-7H-pyrrolo[2,3-d]pyrimidin-2-amine
Compound IV: (4E,6Z,10E)-19-(allylamino)-13-hydroxy-8,14-dimethoxy-4,10,12,16-tetramethyl-3,20,22-trioxo-2-azabicyclo[16.3.1]docosa-1(21),4,6,10,18-pentaen-9-yl carbamate
Compound V: (4E,6Z,8S,9S,10E,12S,13R,14S,16R)-19-(2-(dimethylamino)ethylamino)-13-hydroxy-8,14-dimethoxy-4,10,12,16-tetramethyl-3,20,22-trioxo-2-azabicyclo[16.3.1]docosa-1(21),4,6,10,18-pentaen-9-yl carbamate
Compound VI: (1aS,2Z,4E,14R,15aS)-8-chloro-9,11-dihydroxy-14-methyl-15,15a-dihydro-1aH-benzo[c]oxireno[2,3-k][1]oxacyclotetradecine-6,12(7H,14H)-dione
Compound VII: 6-chloro-9-((4-methoxy-3,5-dimethylpyridin-2-yl)methyl)-9H-purin-2-amine
Compound VIII: 6-chloro-9-((5-methoxy-4,6-dimethylpyridin-3-yl)methyl)-9H-purin-2-amine
Compound IX: 4-(2-amino-4-chloro-7-((4-methoxy-3,5-dimethylpyridin-2-yl)methyl)-7H-pyrrolo[2,3-d]pyrimidin-5-yl)but-3-yn-1-ol
Compound X: 5-(2-amino-4-chloro-7-((4-methoxy-3,5-dimethylpyridin-2-yl)methyl)-7H-pyrrolo[2,3-d]pyrimidin-5-yl)-1-(4-methylpiperazin-1-yl)pent-4-yn-1-one
Compound XI: 5-(2-amino-4-chloro-7-((4-methoxy-3,5-dimethylpyridin-2-yl)methyl)-7H-pyrrolo[2,3-d]pyrimidin-5-yl)pent-4-ynamide
Compound XII: 3-(2-amino-4-chloro-7-((4-methoxy-3,5-dimethylpyridin-2-yl)methyl)-7H-pyrrolo[2,3-d]pyrimidin-5-yl)propan-1-ol
Compound XIII: 5-(2-amino-4-chloro-7-((4-methoxy-3,5-dimethylpyridin-2-yl)methyl)-7H-pyrrolo[2,3-d]pyrimidin-5-yl)pent-4-yn-2-ol
Compound XIV: 5-(2-amino-4-chloro-7-((4-methoxy-3,5-dimethylpyridin-2-yl)methyl)-7H-pyrrolo[2,3-d]pyrimidin-5-yl)-2-methylpent-4-yn-2-ol
Compound XV: 4-(4-(2-amino-4-chloro-7-((4-methoxy-3,5-dimethylpyridin-2-yl)methyl)-7H-pyrrolo[2,3-d]pyrimidin-5-yl)but-3-ynyl)-N-methylpiperazine-1-carboxamide
Compound XVI: 5-(5-(1H-imidazol-1-yl)pent-1-ynyl)-4-chloro-7-((4-methoxy-3,5-dimethylpyridin-2-yl)methyl)-7H-pyrrolo[2,3-d]pyrimidin-2-amine

### BIOLOGY EXAMPLES

### Example A

rHSP90 Competitive Binding Assay

Five microgram of purified rHSP90 protein (Stressgen, BC, Canada, #SPP-770) in phosphate buffered saline (PBS) was coated on 96 well plates by incubating overnight at 4° C. Unbound protein was removed and the coated wells were washed twice with 200 μL PBS. DMSO controls (considered as untreated samples) or test compounds were then added at 100-30-10-3-1-0.3 μM dilutions (in PBS), the plates mixed for 30 seconds on the plate shaker, and then incubated for 60 mm. at 37° C. The wells were washed twice with 200 μL PBS, and 10 μM biotinylated-geldanamycin (biotin-GM) was added and incubated for 60 min. at 37° C. The wells were washed again twice with 200 μL PBS, before the addition of 20 μg/mL streptavidin-phycoerythrin (streptavidin-PE) (Molecular Probes, Eugene, Oreg.) and incubation for 60 min. at 37° C. The wells were washed again twice with 200 μL PBS. Relative fluorescence units (RFU) was measured using a SpectraMax Gemini XS Spectrofluorometer (Molecular Devices, Sunnyvale, Calif.) with an excitation at 485 nm and emission at 580 nm; data was acquired using SOFTmax®PRO software (Molecular Devices Corporation, Sunnyvale, Calif.). The background was defined as the RFU generated from wells that were not coated with HSP90 but were treated with the biotin-GM and streptavidin-PE. The background measurements were subtracted from each sample treated with biotin-GM and streptavidin-PE measurements before other computation. Percent inhibition of binding for each sample was calculated from the background subtracted values as follows:
% binding inhibition=[(RFU untreated−RFU treated)/RFU untreated]×100.

### Example B

Cell Lysate Binding Assay

MCF7 breast carcinoma cell lysates were prepared by douncing in lysing buffer (20 mM HEPES, pH 7.3, 1 mM EDTA, 5 mM MgCl₂, 100 mM KCI), and then incubated with or without test compound for 30 mins at 4° C., followed by incubation with biotin-GM linked to BioMag™ streptavidin magnetic beads (Qiagen) for 1 hr at 4° C. The tubes were placed on a magnetic rack, and the unbound supernatant removed. The magnetic beads were washed three times in lysis buffer and boiled for 5 mins at 95° C. in SDS-PAGE sample buffer. Samples were analyzed on SDS protein gels, and Western Blots were done for rHSP90. Bands in the Western Blots were quantitated using the Bio-rad Fluor-S Multilmager, and the % inhibition of binding of rHSP90 to the biotin-GM was calculated.

### Example C

Her2 Degradation Assay

MCF7 breast carcinoma cells (ATCC) were grown in Dulbecco's modified Eagle's medium (DMEM) containing 10% fetal bovine serum (FBS) and 10 mM HEPES, and plated in 24 well plates (50% confluent). Twenty-four hrs later (cells are 65-70% confluent), test compounds were added and incubated overnight for 16 h. The wells were washed with 1 mL phosphate buffered saline (PBS), and 200 μL trypsin was added to each well. After trypsinization was complete, 50 μL of FBS was added to each well. Then 200 mL cells was transferred to 96 well plates. The cells were pipetted up and down to obtain a single cell suspension. The plates were centrifuged at 2,500 rpm for 1 min using a Sorvall Legend RT™ tabletop centrifuge (Kendro Laboratory Products, Asheville, N.C.). The cells were then washed once in PBS containing 0.2% BSA and 0.2% sodium azide (BA buffer). Phycoerythrin (PE) conjugated anti HER2/Neu antibody (Becton Dickinson, #340552), or PE conjugated anti-keyhole limpet hemocyanin [KLHJ (Becton Dickinson, #340761) control antibody was added at a dilution of 1:20 and 1:40 respectively (final concentration was 1 μg/mL) and the cells were pipetted up and down to form a single cell suspension, and incubated for 15 mins. The cells were washed twice with 200 μL BA buffer, and resuspended in 200 μL BA buffer, and transferred to FACSCAN tubes with an additional 250 μL BA buffer. Samples were analyzed using a FACSCalibur™ flow cytometer (Becton Dickinson, San Jose, Calif.) equipped with Argon-ion laser that emits 15 mW of 488 nm light for excitation of the PE fluorochrome. 10,000 events were collected per sample. A fluorescence histogram was generated and the mean fluorescence intensity (MFI) of each sample was determined using Celiquest software. The background was defined as the MFI generated from cells incubated with control IgG-PE, and was subtracted from each sample stained with the HER2/Neu antibody. Cells incubated with DMSO were used as untreated controls since the compounds were resuspended in DMSO. Percent degradation of Her2 was calculated as follows:
% Her2 degraded=[(MF1 untreated cells−MF1 treated cells)/MF1 untreated cell]×100

### Example D

MTS Assay

MTS assays measure the cytotoxicity of the compounds. MTS (3-(4,5-dimethylthiazol-2-yl)-5-(3-carboxymethoxyphenyl)-2-(4-sulfophenyl)-2H-tetrazolium) is a tetrazolium dye that is converted to a formazan product by dehydrogenase enzymes of metabolically active cells (Corey, A. et al. “Use of an aqueous soluble tetrazoliumlformazan assay for cell growth assays in culture,” Cancer Commun. 1991, 3, 207-2 12). Cells were seeded in 96 well plates at 2000 cells/well and allowed to adhere overnight in Dulbecco's modified Eagle's medium supplemented with 10% fetal bovine serum. The final culture volume was 100 μl. Viable cell number was determined by using the Celltiter 96 AQᵤₑₒᵤₛ Non-radioactive Cell Proliferation Assay (Promega, Madison Wis.). The MTS/PMS (phenazine methosulfate) solution was mixed at a ratio of 20:1, and 20 μL was added per well to 100 μl of culture medium. After 2-4 hours, the formation of the formazan product was measured at 490 nm absorbance using a multiwell plate spectrophotometer. Background was determined by measuring the Abs 490 nm of cell culture medium and MTS-PMS in the absence of cells and was subtracted from all values. Percent viable cells was calculated as follows:
% viable cells=(Abs at 490 nm treated cells/Abs at 490 nm untreated cells)×100

What is claimed is:

1. A method of treating an individual having an HSP90 mediated disorder comprising selecting a synthetic heterocyclic HSP 90 inhibitor wherein the activity of the HSP90 inhibitor is substantially independent of multi drug resistance, and administering to said individual a pharmaceutical composition comprising a pharmaceutically effective amount of said synthetic heterocyclic HSP90 inhibitor.

2. The method of claim 1, wherein the selecting step comprises determining whether said selected synthetic heterocyclic HSP90 inhibitor is a substrate of one or more MDR family members.

3. The method of claim 1, wherein said selected synthetic heterocyclic HSP90 inhibitor is a substrate of P-gp.

4. The method of claim 1 wherein the HSP90 inhibiting activity of said selected synthetic heterocyclic HSP90 inhibitor in cells without MDR expression is substantially the same as the inhibiting activity of said selected synthetic heterocyclic HSP90 inhibitor in cells with MDR expression.

5. The method of claim 1 wherein the ratio of the HSP90 IC50 of said selected synthetic heterocyclic HSP90 inhibitor in cells without MDR expression and cells with MDR expression is less than 1:4.

6. The method of claim 1 wherein the ratio of the HSP90 IC50 of said selected synthetic heterocyclic HSP90 inhibitor in cells without MDR expression and cells with MDR expression is less than 1:3.

7. The method of claim 1 wherein the ratio of the HSP90 IC50 of said selected synthetic heterocyclic HSP90 inhibitor in cells without MDR expression and with MDR expression is less than 1:2.

8. The method of claim 1 wherein the ratio of the HSP90 IC50 of said selected synthetic heterocyclic HSP90 inhibitor in cells without MDR expression and cells with MDR expression is less than 1:1.5.

9. The method of claim 1 wherein the ratio of the HSP90 IC50 of said selected synthetic heterocyclic HSP90 inhibitor in cells without MDR expression and cells with MDR expression is about 1:1.

10. The method of claim 1, wherein said selected synthetic heterocyclic HSP90 inhibitor inhibitor is a purine analog.

11. The method of claim 1, wherein said selected synthetic heterocyclic HSP90 inhibitor inhibitor is selected from the group consisting of 2-aminopurines, pyrazolopyridines, pyrrolopyrimidines, triazopyrimides and alkynyl pyrrolopyrimidines.

12. The method of claim 1, wherein the HSP90 mediated disorder is selected from the group of inflammatory diseases, infections, autoimmune disorders, stroke, ischemia, cardiac disorder, neurological disorders, fibrogenetic disorders selected from the group of scleroderma, polymyositis, systemic lupus, rheumatoid arthritis, liver cirrhosis, keloid formation, interstitial nephritis and pulmonary fibrosis, proliferative disorders, tumors, leukemias, neoplasms, cancers, carcinomas, metabolic diseases, and malignant disease.

13. The method of claim 1, wherein the HSP90 mediated disorder is a cancer located in organs protected by P-gp.

14. The method of claim 1, wherein the HSP90 mediated disorder is a cancer located in adrenal glands, brain, heart, liver, kidneys, uterus, and testis.

15. The method of claim 1, wherein the HSP90 mediated disorder is a cancer in which P-gp is substantially expressed in cancer cells.

16. The method of claim 1, wherein the HSP90 mediated disorder is colon, kidney, adrenocortical, and hepatocellular cancer. 16. The method of claim 1, further comprising administering at least one therapeutic agent selected from the group of cytotoxic agents, anti-angiogenesis agents and anti-neoplastic agents wherein the at least one anti-neoplastic agent is selected from the group of alkylating agents, anti-metabolites, epidophyllotoxins; antineoplastic enzymes, topoisomerase inhibitors, procarbazines, mitoxantrones, platinum coordination complexes, biological response modifiers and growth inhibitors, hormonal/anti-hormonal therapeutic agents, and haematopoietic growth factors.

17. The method of claim 1 wherein said selected synthetic heterocyclic HSP90 inhibitor has Formula A, tautomer, pharmaceutically acceptable salt thereof, or prodrug thereof 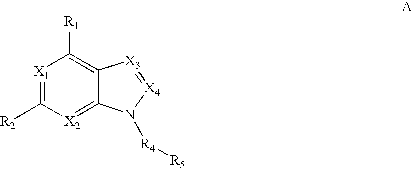
or tautomer or pharmaceutically acceptable salt or prodrug thereof, wherein
X₁ and X₂ are the same or different and each is nitrogen or —CR⁶;
X₃ is nitrogen or —CR³ wherein R³ is hydrogen, OH, a keto tautomer, —OR⁸, —CN, halogen, lower alkyl, or —C(O)R⁹;
X₄ is nitrogen or a group CR⁶ when X₃ is nitrogen, and X₄ is —CR⁶R⁷ when X₃ is —CR³;
R¹ is halogen, —OR⁸, —SR⁸, or lower alkyl;
R² is —NR⁸R¹⁰;
R⁴ is —(CH₂)ₙ— where n=0-3; and
R⁵ is alkyl, aryl, heteroaryl, alicyclic, heterocyclic, all optionally bi- or tricyclic, and optionally substituted with H, halogen, lower alkyl, lower alkenyl, lower alkynyl, lower aryl, lower alicyclic, aralkyl, aryloxyalkyl, alkoxyalkyl, perhaloalkyl, perhaloalkyloxy, perhaloacyl, —N₃, —SR⁸, —OR⁸, —CN, —CO₂R⁹, —NO₂, or —NR⁸R¹⁰;
R⁸ is hydrogen, lower alkyl, lower aryl, or —(CO)R⁹;
R⁹ is lower alkyl, lower aryl, lower heteroaryl, —NR⁸R¹⁰ or —OR¹¹;
R¹¹ is lower alkyl or lower aryl; and
R¹⁰ is hydrogen or lower alkyl.

18. The method of claim 1 wherein said selected synthetic heterocyclic HSP90 inhibitor has Formula II, tautomer, pharmaceutically acceptable salt thereof, or prodrug thereof: 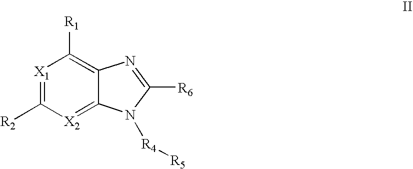
wherein
X₁ and X₂ are the same or different and each is nitrogen or —CR⁶;
R¹ is halogen, OR⁸, SR⁸, or lower alkyl;
R² is —NR⁸R¹⁰;
R⁴ is —(CH₂)ₙ— where n=0-3;
R⁶ is hydrogen, halogen, lower alkyl, —SR⁸, —OR⁸, —NR⁸R¹⁰, —N₃, —CN, or —C(O)R⁹;
R⁵ is alkyl, aromatic, heteroaromatic, alicyclic, or heterocyclic, all optionally bi- or tri-cyclic, and all optionally substituted with H, halogen, lower alkyl, —SR⁸, —OR⁸, —CN, —CO₂R⁹, —NO₂, or —NR⁸R¹⁰;
R⁸ is hydrogen, lower alkyl, lower aryl, or —(CO)R⁹;
R⁹ is lower alkyl, lower aryl, lower heteroaryl, —NR⁸R¹⁰ or —OR¹¹;
R¹¹ is lower alkyl or lower aryl; and
R¹⁰ is hydrogen or lower alkyl.

19. The method of claim 1 wherein said selected synthetic heterocyclic HSP90 inhibitor has Formula IV, tautomer, pharmaceutically acceptable salt thereof, or prodrug thereof: 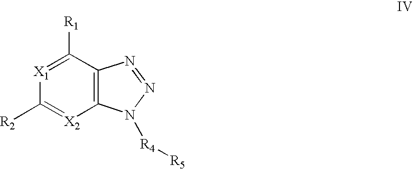
wherein
X₁ and X₂ are the same or different and each is nitrogen or CR⁶;
R¹ is halogen, —OR⁸, —SR⁸, or lower alkyl;
R² is —NR⁸R¹⁰;
R⁴ is —(CH2)ₙ,— where n=0-3;
R⁵ is alkyl, aryl, heteroaryl, alicyclic, heterocyclic, all optionally bi- or tricyclic, and all optionally substituted with H, halogen, lower alkyl, —SR⁸, —OR⁸, —CN, —CO₂R⁹, —NO₂, or —NR⁸R¹⁰;
R⁸ is hydrogen, lower alkyl, lower aryl or —(CO)R⁹;
R⁹ is lower alkyl, lower aryl, lower heteroaryl, —NR⁸R¹⁰ or —OR¹¹;
R¹¹ is lower alkyl or lower aryl; and
R¹⁰ is hydrogen or lower alkyl.

20. The method of claim 1 wherein said selected synthetic heterocyclic HSP90 inhibitor has Formula III, tautomer, pharmaceutically acceptable salt thereof, or prodrug thereof: 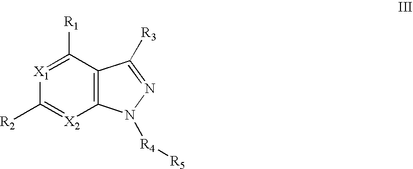
wherein
X₁ and X₂ are the same or different and each is nitrogen or CR⁶;
R₁ is halogen, —OR⁸, —SR⁸ or lower alkyl;
R² is —NR⁸R¹⁰;
R³ is hydrogen, OH or keto tautomer, —OR⁸, halogen, —CN, lower alkyl or —C(O)R⁹;
R⁴ is —(CH₂)ₙ— where n=0-3;
R⁵ is alkyl, aryl, heteroaryl, alicyclic, heterocyclic, all optionally bi- or tricyclic, and all optionally substituted with H, halogen, lower alkyl, —SR⁸, —OR⁸, —CN, —CO₂R⁹, —NO₂ or —NR⁸R¹⁰;
R⁸ is hydrogen, lower alkyl, lower aryl or —(CO)R⁹;
R⁹ is lower alkyl, lower aryl, lower heteroaryl, —NR⁸R¹⁰ or OR¹¹;
R¹¹ is lower alkyl or lower aryl; and
R¹⁰ is hydrogen or lower alkyl.

21. The method of claim 1 wherein said selected synthetic heterocyclic HSP90 inhibitor has Formula F, tautomer, pharmaceutically acceptable salt thereof, or prodrug thereof: 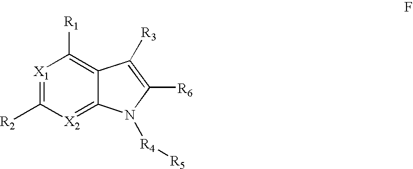
wherein
X₁ and X₂ are the same or different and each is nitrogen or —CR⁶;
R₁ is halogen, —OR⁸, —SR⁸ or lower alkyl;
R² is —NR⁸R¹⁰;
R³ is hydrogen, OH or a keto tautomer, —OR⁸, halogen, —CN, lower alkyl or —C(O)R⁹;
R⁴ is —(CH₂)ₙ— where n=0-3;
R⁶ is hydrogen, halogen, lower alkyl, —SR⁸, —OR⁸, —NR⁸R¹⁰, —N₃, or —C(O)R⁹;
R⁵ is alkyl, aromatic, heteroaromatic, alicyclic, heterocyclic, all optionally bi- or tricyclic, and all optionally substituted with H, halogen, lower alkyl, —SR⁸, —OR⁸, —CN, —CO₂R⁹, —NO₂, or —NR⁸R¹⁰;
R⁸ is hydrogen, lower alkyl, lower aryl, or —(CO)R⁹;
R⁹ is lower alkyl, lower aryl, lower heteroaryl, —NR⁸R¹⁰ or —OR¹¹;
R¹¹ is lower alkyl or lower aryl; and
R¹⁰ is hydrogen or lower alkyl;

22. The method of claim 1 wherein said selected synthetic heterocyclic HSP90 inhibitor has Formula IIA, tautomer, pharmaceutically acceptable salt thereof, or prodrug thereof: 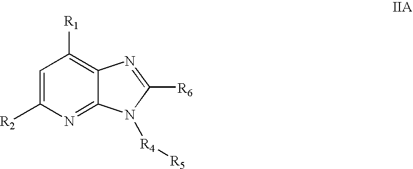
wherein
R₁ is halogen, —OR⁸, —SR⁸ or lower alkyl;
R² is —NR⁸R¹⁰;
R⁴ is —(CH₂)ₙ— where n=0-3;
R⁶ is hydrogen, halogen, lower alkyl, —SR⁸, —OR⁸, —NR⁸R¹⁰, —N₃, —CN or C(O)R⁹;
R⁵ is alkyl, aromatic, heteroaromatic, alicyclic, heterocyclic, all optionally bi- or tricyclic, and all optionally substituted with H, halogen, lower alkyl, —SR⁸, —OR⁸, —CN, —CO₂R⁹, —NO₂, or —NR⁸R¹⁰;
R⁸ is hydrogen, lower alkyl, lower aryl, or —(CO)R⁹;
R⁹ is lower alkyl, lower aryl, lower heteroaryl, NR⁸R¹⁰ or OR¹¹;
R¹¹ is lower alkyl or lower aryl; and
R¹⁰ is hydrogen or lower alkyl.

23. The method of claim 1 wherein said selected synthetic heterocyclic HSP90 inhibitor has Formula IIB, tautomer, pharmaceutically acceptable salt thereof, or prodrug thereof: 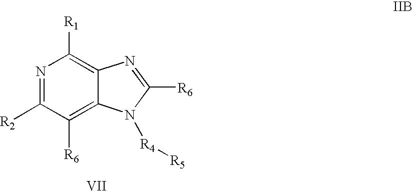
wherein
R¹ is halogen, —OR⁸, —SR⁸ or lower alkyl;
R² is —NR⁸R¹⁰;
R⁴ is —(CH₂)ₙ—, where n=0-3;
R⁶ is hydrogen, halogen, lower alkyl, —SR⁸, —OR⁸, —NR⁸R¹⁰, —N₃, —CN or —C(O)R⁹;
R⁵ is alkyl, aromatic, heteroaromatic, alicyclic, heterocyclic, all optionally bi- or tricyclic, and all optionally substituted with H, halogen, lower alkyl, —SR⁸, —OR⁸, —CN, —CO₂R⁹, —NO₂, or —NR⁸R¹⁰;
R⁸ is hydrogen, lower alkyl, lower aryl, or —(CO)R⁹;
R⁹ is lower alkyl, lower aryl, lower heteroaryl, —NR⁸R¹⁰ or —OR¹¹;
R¹¹ is lower alkyl or lower aryl; and
R¹⁰ is hydrogen or lower alkyl.

24. The method of claim 1 wherein said selected synthetic heterocyclic HSP90 inhibitor has Formula IA, tautomer, pharmaceutically acceptable salt thereof, or prodrug thereof: 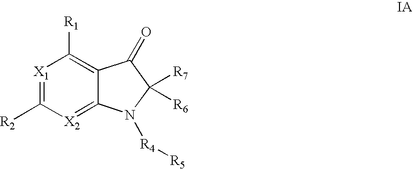
wherein:
X₁ and X₂ are the same or different and each is nitrogen or a group —CR⁶;
R¹ is halogen, —OR⁸, —SR⁸, or lower alkyl;
R² is —NR⁸R¹⁰;
R⁴ is —(CH₂)ₙ— where n=0-3;
R⁵ is alkyl, aromatic, heteroaromatic, alicyclic, heterocyclic, all optionally bi- or tricyclic, and all optionally substituted with H, halogen, lower alkyl, —SR⁸, —OR⁸, —CN, —CO₂R⁹, —NO₂, or —NR⁸R¹⁰;
R⁶ is hydrogen, halogen, lower alkyl, —SR⁸, —OR⁸, —NR⁸R¹⁰, —N₃, —CN, —C(O)R⁹, or together with R⁷ is carbonyl (C═O);
R⁷ is independently selected from hydrogen, lower alkyl or together with R⁶ is carbonyl (C═O);
R⁸ is hydrogen, lower alkyl, lower aryl, or —(CO)R⁹;
R⁹ is lower alkyl, lower aryl, lower heteroaryl, —NR⁸R¹⁰ or —OR¹¹; R¹¹ is lower alkyl or lower aryl; and
R¹⁰ is hydrogen or lower alkyl.

25. The method of claim 1 wherein said selected synthetic heterocyclic HSP90 inhibitor has Formula IC, tautomer, pharmaceutically acceptable salt thereof, or prodrug thereof: 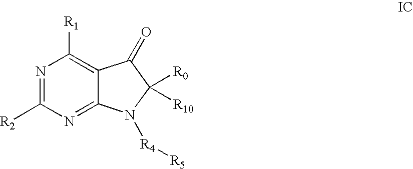
wherein:
R⁰ is hydrogen, halogen, lower alkyl, —SR⁸, —OR⁸, —CN or —NHR⁸;
R¹ is halogen, —OR¹¹, —SR¹¹ or lower alkyl;
R² is —NH₂;
R⁴ is —CHR¹²—;
R⁵ is aryl, heteroaryl, alicyclic, or heterocyclic, wherein:
the aryl group is substituted with 3 to 5 substituents,
the heteroaryl group is substituted with 2 to 5 substituents,
the alicyclic group is substituted with 3 to 5 substituents,
the heterocyclic group is substituted with 3 to 5 substituents, and
the substituents on R⁵ are selected from the group consisting of halogen, lower alkyl, lower alkenyl, lower alkynyl, —SR⁸, —OR⁸, —CN, —C(O)OH, —C(O)R⁹, —NO₂, —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein when R⁸ and R¹⁰ taken together optionally form a ring of 3-7 ring atoms and optionally 1-3 of the ring atoms are heteroatoms selected from the group of O, S and N;
R⁸ is hydrogen, lower alkyl, lower alkenyl, lower alkynyl, lower aryl, lower heteroaryl, or —C(O)R⁹;
R⁹ is H, lower alkyl, lower aryl, lower heteroaryl, —NR¹⁰R¹⁰, or —OR¹¹, wherein
R¹⁰ and R¹⁰ taken together optionally form a ring of 3-7 ring atoms and optionally 1-3 of the ring atoms are heteroatoms selected from the group of O, S and N;
R¹⁰ is hydrogen, lower alkyl, lower heteroaryl, lower aryl, lower alkenyl, or lower alkynyl;
R¹¹ is lower alkyl, lower alkenyl, lower alkynyl, lower heteroaryl or lower aryl;
R¹² is hydrogen or lower alkyl; and
R⁰ and R¹⁰ taken together optionally form an exocyclic double bond which is optionally substituted, or optionally form a ring of 3-7 ring atoms and optionally 1-3 of the ring atoms are heteroatoms selected from the group of O, S and N.

26. The method of claim 1 wherein said selected synthetic heterocyclic HSP90 inhibitor has Formula ID, tautomer, pharmaceutically acceptable salt thereof, or prodrug thereof: 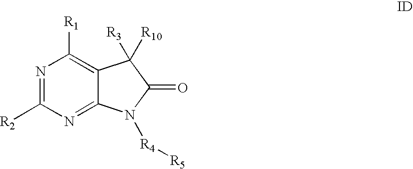
wherein:
R¹ is halogen, —OR¹¹, —SR¹¹ or lower alkyl;
R² is —NH₂;
R³ is selected from the group consisting of hydrogen, halogen, —SR⁸, —OR⁸, —CN, —C(O)R⁹, —C(O)OH, —NO₂, —NR⁸R¹⁰, lower alkyl, lower alkenyl, lower alkynyl, lower perhaloalkyl, aryl, heteroaryl, alicyclic, heterocyclic, all optionally substituted, wherein:
the aryl, heteroaryl, alicyclic and heterocyclic groups are optionally mono-, bi- or tri-cyclic, R⁸ and R¹⁰ taken together optionally form a ring of 3-7 ring atoms and optionally 1-3 of the ring atoms are heteroatoms selected from the group of O, S and N, and
the optional substituents on R³ are selected from the group consisting of halogen, lower alkyl, lower alkenyl, lower alkynyl, —SR⁸, —OR⁸, —CN, —C(O)OH, —C(O)R⁹, —NO₂, —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;
R⁴ is —CHR¹²—;
R⁵ is aryl, heteroaryl, alicyclic, or heterocyclic, wherein
the aryl group is substituted with 3 to 5 substituents,
the heteroaryl group is substituted with 2 to 5 substituents,
the alicyclic group is substituted with 3 to 5 substituents,
the heterocyclic group is substituted with 3 to 5 substituents, and
the substituents on R⁵ are selected from the group consisting of halogen, lower alkyl, lower alkenyl, lower alkynyl, —SR⁸, —OR⁸, —CN, —C(O)OH, —C(O)R⁹, —NO₂, —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;
R⁸ is hydrogen, lower alkyl, lower alkenyl, lower alkynyl, lower aryl, lower heteroaryl, or —C(O)R⁹;
R⁹ is H, lower alkyl, lower aryl, lower heteroaryl, —NR¹⁰R¹⁰, or —OR¹¹, wherein when R¹⁰ and R¹⁰ taken together optionally form a ring of 3-7 ring atoms and optionally 1-3 of the ring atoms are heteroatoms selected from the group of O, S and N;
R¹⁰ is hydrogen, lower alkyl, lower heteroaryl, lower aryl, lower alkenyl, or lower alkynyl,
R¹¹ is lower alkyl, lower alkenyl, lower alkynyl, lower heteroaryl or lower aryl;
R¹² is hydrogen or lower alkyl; and
R³ and R¹⁰ taken together optionally form an exocyclic double bond which is optionally substituted, or optionally form a ring of 3-7 ring atoms and optionally 1-3 of the ring atoms are heteroatoms selected from the group of O, S and N.

27. The method of claim 1 wherein said selected synthetic heterocyclic HSP90 inhibitor has Formula IE, tautomer, pharmaceutically acceptable salt thereof, or prodrug thereof: 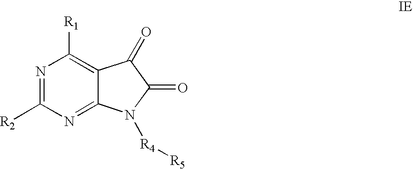
wherein:
R¹ is halogen, —OR¹¹, —SR¹¹ or lower alkyl;
R² is —NH₂;
R⁴ is —CHR¹²—;
R⁵ is aryl, heteroaryl, alicyclic, or heterocyclic, wherein
the aryl group is substituted with 3 to 5 substituents,
the heteroaryl group is substituted with 2 to 5 substituents,
the alicyclic group is substituted with 3 to 5 substituents,
the heterocyclic group is substituted with 3 to 5 substituents, and
the substituents on R⁵ are selected from the group consisting of halogen, lower alkyl, lower alkenyl, lower alkynyl, —SR⁸, —OR⁸, —CN, —C(O)OH, —C(O)R⁹, —NO₂, —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;
R⁸ is hydrogen, lower alkyl, lower alkenyl, lower alkynyl, lower aryl, lower heteroaryl, or —C(O)R⁹;
R⁹ is H, lower alkyl, lower aryl, lower heteroaryl, —NR¹⁰R¹⁰, or —OR¹¹, wherein R¹⁰ and R¹⁰ taken together optionally form a ring of 3-7 ring atoms and optionally 1-3 of the ring atoms are heteroatoms selected from the group of O, S and N;
R¹⁰ is hydrogen, lower alkyl, lower heteroaryl, lower aryl, lower alkenyl, or lower alkynyl,
R¹¹ is lower alkyl, lower alkenyl, lower alkynyl, lower heteroaryl or lower aryl; and
R¹² is hydrogen or lower alkyl.

28. The method of claim 1 wherein said selected synthetic heterocyclic HSP90 inhibitor has Formula IIC, tautomer, pharmaceutically acceptable salt thereof, or prodrug thereof: 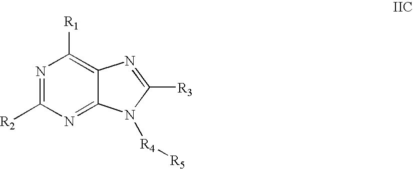
wherein:
R¹ is halogen or lower alkyl;
R² is —NR⁸R¹⁰;
R⁴ is —CHR¹²—;
R³ is hydrogen, halogen, or —CN;
R⁵ is aryl, heteroaryl, alicyclic, or heterocyclic, wherein:
the aryl group is substituted with 3 to 5 substituents,
the heteroaryl group is substituted with 2 to 5 substituents, wherein when the heteroaryl is substituted with only two substituents, the two substituents must form part of an optionally substituted fused ring,
the alicyclic group is substituted with 3 to 5 substituents,
the heterocyclic group is substituted with 3 to 5 substituents, and
the substituents are selected from the group of halogen, lower alkyl, lower alkenyl, lower alkynyl, lower aryl, lower alicyclic, arylalkyl, aryloxy, aryloxyalkyl, alkoxyalkyl, perhaloalkyl, perhaloalkyloxy, perhaloacyl, —N₃, —SR⁸, —OR⁸, —CN, —C(O)R⁹, —NO₂, —NR⁸R¹⁰, phosphonate and phosphonic acid;
R⁸ is hydrogen, lower alkyl, lower aryl, or —C(O)R⁹;
R⁹ is lower alkyl, lower aryl, lower heteroaryl, —NR¹⁰R¹⁰ or —OR¹¹;
R¹⁰ is independently hydrogen or lower alkyl;
R¹¹ is lower alkyl, lower aryl or lower heteroaryl;
R¹² is hydrogen or lower alkyl;
provided that
when R⁵ is aryl, R⁵ is not an organo-metallic cyclopentadiene;
when R⁵ is phenyl, the substituents are not 3,5 di-halo;
when R⁵ is alicyclic, the ring system does not contain any tetra-substituted sp³ ring carbons; and
when R⁵ is heterocyclic, the ring system does not contain any tetra-substituted sp³ ring carbons or the ring system is not a tetra-substituted pyrrolidine.

29. The method of claim 1 wherein said selected synthetic heterocyclic HSP90 inhibitor has Formula IID, tautomer, pharmaceutically acceptable salt thereof, or prodrug thereof: 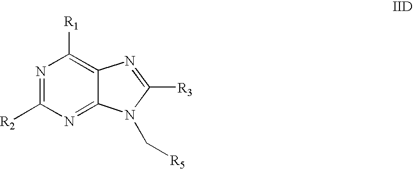
wherein:
R¹ is halogen or lower alkyl;
R² is —NR⁸R¹⁰;
R³ is hydrogen, halogen, or —CN;
R⁵ is aryl, heteroaryl, alicyclic, or heterocyclic, wherein
the aryl group is substituted with 3 to 5 substituents,
the heteroaryl group is substituted with 2 to 5 substituents, wherein when the heteroaryl is substituted with only two substituents, the two substituents must form part of an optionally substituted fused ring,
the alicyclic group is substituted with 3 to 5 substituents,
the heterocyclic group is substituted with 3 to 5 substituents, and
the substituents are selected from the group of halogen, lower alkyl, lower alkenyl, lower alkynyl, lower aryl, lower alicyclic, arylalkyl, aryloxy, aryloxyalkyl, alkoxyalkyl, perhaloalkyl, perhaloalkyloxy, perhaloacyl, —N₃, —SR⁸, —OR⁸, —CN, —C(O)R⁹, —NO₂, —NR⁸R¹⁰, phosphonate and phosphonic acid;
R⁸ is hydrogen, lower alkyl, lower aryl, or —C(O)R⁹;
R⁹ is lower alkyl, lower aryl, lower heteroaryl, —NR¹⁰R¹⁰ or —OR¹¹;
R¹⁰ is independently hydrogen or lower alkyl; and
R¹¹ is lower alkyl, lower aryl or lower heteroaryl;
provided that
when R⁵ is aryl, R⁵ is not an organo-metallic cyclopentadiene;
when R⁵ is phenyl, the substituents are not 3,5 di-halo;
when R⁵ is alicyclic, the ring system does not contain any tetra-substituted sp³ ring carbons;
when R⁵ is heterocyclic, the ring system does not contain any tetra-substituted sp³ ring carbons or the ring system is not a tetra-substituted pyrrolidine.

30. The method of claim 1 wherein said selected synthetic heterocyclic HSP90 inhibitor has Formula IIIA, tautomer, pharmaceutically acceptable salt thereof, or prodrug thereof: 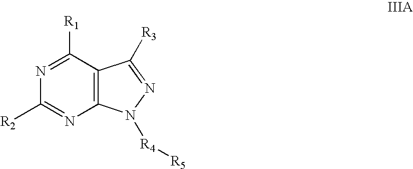
wherein:
R¹ is halogen, —OR¹¹, —SR¹¹ or lower alkyl;
R² is —NHR⁸;
R³ is selected from the group consisting of hydrogen, halogen, —SR⁸, —OR⁸, —CN, —C(O)R⁹, —CO₂H, —NO₂, —NR⁸R¹⁰, lower alkyl, lower alkenyl, lower alkynyl, lower perhaloalkyl, aryl, heteroaryl, alicyclic and heterocyclic, all optionally substituted, wherein:
the aryl, heteroaryl, alicyclic and heterocyclic groups are optionally mono-, bi- or tri-cyclic,
R⁸ and R¹⁰ taken together optionally form a ring of 3-7 ring atoms and optionally 1-2 of the ring atoms are heteroatoms selected from the group of O, S and N, and
the optional substituents on R³ are selected from the group consisting of halogen, lower alkyl, lower alkenyl, lower alkynyl, —SR⁸, —OR⁸, —CN, —C(O)R⁹, —C(O)OH, —NO₂, —NR⁸R¹⁰, lower aryl, lower heteroaryl, lower alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;
R⁴ is —CHR¹²—;
R⁵ is aryl, heteroaryl, alicyclic, or heterocyclic, wherein
the aryl group is substituted with 3 to 5 substituents,
the heteroaryl group is substituted with 2 to 5 substituents,
the alicyclic group is substituted with 3 to 5 substituents,
the heterocyclic group is substituted with 3 to 5 substituents, and
the substituents on R⁵ are selected from the group consisting of halogen, lower alkyl, lower alkenyl, lower alkynyl, —SR⁸, —OR⁸, —CN, —C(O)OH, —C(O)R⁹, —NO₂ and —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;
R⁸ is hydrogen, lower alkyl, lower alkenyl, lower alkynyl, lower aryl, lower heteroaryl or —C(O)R⁹;
R⁹ is hydrogen, lower alkyl, lower alkenyl, lower alkynyl, lower aryl, lower heteroaryl, —NR¹⁰R¹⁰ or —OR¹¹, R¹⁰ and R¹⁰ taken together optionally form a ring of 3-7 ring atoms and optionally 1-3 of the ring atoms are heteroatoms selected from the group of O, S and N;
R¹⁰ is hydrogen, lower alkyl, lower alkenyl, lower alkynyl, lower aryl or lower heteroaryl;
R¹¹ is lower alkyl, lower alkenyl, lower alkynyl, lower aryl or lower heteroaryl; and
R¹² is hydrogen or lower alkyl.

31. The method of claim 1 wherein said selected synthetic heterocyclic HSP90 inhibitor has Formula IIIB, tautomer, pharmaceutically acceptable salt thereof, or prodrug thereof: 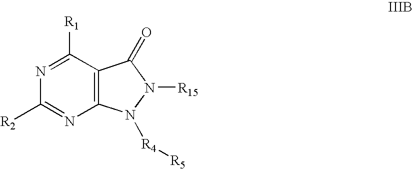
wherein:
R¹ is halogen, —OR¹¹, —SR¹¹ or lower alkyl;
R² is —NHR⁸;
R⁴ is —CHR¹²—;
R⁵ is aryl, heteroaryl, alicyclic, or heterocyclic, wherein
the aryl group is substituted with 3 to 5 substituents,
the heteroaryl group is substituted with 2 to 5 substituents,
the alicyclic group is substituted with 3 to 5 substituents,
the heterocyclic group is substituted with 3 to 5 substituents, and
the substituents on R⁵ are selected from the group consisting of halogen, lower alkyl, lower alkenyl, lower alkynyl, —SR⁸, —OR⁸, —CN, —C(O)OH, —C(O)R⁹, —NO₂ and —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;
R⁸ is hydrogen, lower alkyl, lower alkenyl, lower alkynyl, lower aryl, lower heteroaryl or —C(O)R⁹;
R⁹ is hydrogen, lower alkyl, lower alkenyl, lower alkynyl, lower aryl, lower heteroaryl, —NR¹⁰R¹⁰ or —OR¹¹, R¹⁰ and R¹⁰ taken together optionally form a ring of 3-7 ring atoms and optionally 1-3 of the ring atoms are heteroatoms selected from the group of O, S and N;
R¹⁰ is hydrogen, lower alkyl, lower alkenyl, lower alkynyl, lower aryl or lower heteroaryl;
R¹¹ is lower alkyl, lower alkenyl, lower alkynyl, lower aryl or lower heteroaryl;
R¹² is hydrogen or lower alkyl; and
R¹⁵ is hydrogen, lower alkyl, lower alkenyl or lower alknyl.

32. The method of claim 1 wherein said selected synthetic heterocyclic HSP90 inhibitor has Formula IVA, tautomer, pharmaceutically acceptable salt thereof, or prodrug thereof: 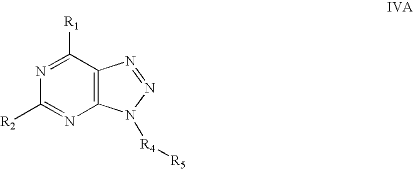
wherein:
R¹ is halogen, —OR¹¹, —SR¹¹ or lower alkyl;
R² is —NHR⁸;
R⁴ is —CHR¹²—;
R⁵ is aryl, heteroaryl, alicyclic, or heterocyclic, wherein:
the aryl group is substituted with 3 to 5 substituents,
the heteroaryl group is substituted with 2 to 5 substituents,
the alicyclic group is substituted with 3 to 5 substituents,
the heterocyclic group is substituted with 3 to 5 substituents, and
the substituents are selected from the group consisting of halogen, lower alkyl, lower alkenyl, lower alkynyl, —SR⁸, —OR⁸, —CN, —C(O)OH, —C(O)R⁹, —NO₂ and —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazol, phosphonates, phosphates, phosphoramides, sulfonates, sulfones, sulfates, sulphonamides, carbamates, ureas, thioureas, thioamides, wherein R⁸ and R¹⁰ taken together optionally form a ring of 3-7 ring atoms and optionally 1-3 of the ring atoms are heteroatoms selected from the group of O, S and N;
R⁸ is hydrogen, lower alkyl, lower alkenyl, or lower alkynyl, lower aryl, lower heteroaryl, or —C(O)R⁹;
R⁹ is H, lower alkyl, lower alkenyl, or lower alkynyl, lower aryl, lower heteroaryl, —NR¹⁰R¹⁰, or —OR¹¹, wherein R¹⁰ and R¹⁰ taken together optionally form a ring of 3-7 ring atoms and optionally 1-3 of the ring atoms are heteroatoms selected from the group of O, S and N;
R¹⁰ is hydrogen, lower alkyl, lower heteroaryl, lower aryl, lower alkenyl, or lower alkynyl,
R¹¹ is lower alkyl, lower alkenyl, lower alkynyl, lower heteroaryl or lower aryl;
and R⁴ is —CHR¹²—, —C(O)—, —C(S)—, —S(O)— or —SO₂—; and
R¹² is hydrogen or lower alkyl;
provided that when R⁵ is alicyclic, the ring system does not contain any tetra-substituted sp³ ring carbons.

33. The method of claim 1 wherein said selected synthetic heterocyclic HSP90 inhibitor has Formula VI, tautomer, pharmaceutically acceptable salt thereof, or prod rug thereof: 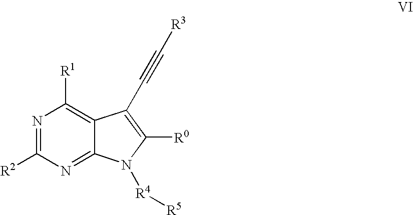
wherein:
R⁰ is selected from the group consisting of hydrogen, halogen, lower alkyl, —CN, —SR⁸, —OR⁸, and —NHR⁸;
R¹ is selected from the group consisting of halogen, —OR¹¹, —SR¹¹ and lower alkyl;
R² is —NHR⁸;
R³ is selected from the group consisting of hydrogen, —CN, —C(O)OH, —OR¹¹, —SR¹¹, —C(O)R⁹, —NR⁸R¹⁰, lower alkyl, lower alkenyl, lower alkynyl, lower perhaloalkyl, lower alkylsilyl, aryl, heteroaryl, alicyclyl and heterocyclyl, all optionally substituted, wherein:
the aryl, heteroaryl, alicyclyl and heterocyclyl groups are mono-, bi- or tri-cyclic;
R⁸ and R¹⁰ taken together with the N atom to which they are attached optionally form an optionally substituted ring comprising 3-7 ring atoms, wherein, in addition to said N atom, 0-3 of the ring atoms are heteroatoms selected from the group consisting of O, S and N;
the optional substituents on R³ are selected from the group consisting of lower alkyl, lower alkenyl, lower alkynyl, —CN, —C(O)OH, —NO₂, —SR⁸, —OR⁸, —C(O)R⁹, —NR⁸R⁸, lower aryl, heteroaryl, alicyclyl, lower heterocyclyl, arylalkyl, heteroarylalkyl, amino, alkylamino, dialkylamino, arylalkylamino, diarylamino, heteroarylamino, diheteroarylamino, arylheteroarylamino, oxo, perhaloalkyl, perhaloalkoxy, perhaloacyl, guanidinyl, pyridinyl, thiophenyl, furanyl, indolyl, indazolyl, phosphonyl, phosphatidyl, phosphoramidyl, sulfanyl, sulfinyl, sulfonyl, sulphonamidyl, carbamyl, uryl, thiouryl and thioamidyl, wherein
R⁸ and R⁸ taken together with the N atom to which they are attached optionally form an optionally substituted ring comprising 3-7 ring atoms, wherein, in addition to said N atom, 0-3 of the ring atoms are heteroatoms selected from the group consisting of O, S and N;
R⁴ is selected from the group consisting of optionally substituted lower alkylene, —C(R¹²)₂—, —C(O)—, —C(S)—, —S(O)— and —SO₂—;
R⁵ is selected from the group consisting of aryl, heteroaryl, alicyclyl and heterocyclyl, wherein:
the aryl group is substituted with 2 to 5 substituents;
the heteroaryl group is substituted with 2 to 5 substituents;
the alicyclyl group is substituted with 3 to 5 substituents;
the heterocyclyl group is substituted with 3 to 5 substituents;
the substituents on R⁵ are selected from the group consisting of halogen, lower alkyl, lower alkenyl, lower alkynyl, —CN, —C(O)OH, —NO₂, —SR⁸, —OR⁸, —C(O)R⁹, —NR⁸R¹⁰, lower aryl, lower heteroaryl, lower alicyclyl, lower heterocyclyl, arylalkyl, heteroarylalkyl, thioalkyl, amino, alkylamino, dialkylamino, arylalkylamino, oxo, perhaloalkyl, perhaloalkoxy, perhaloacyl, guanidinyl, pyridinyl, thiophenyl, furanyl, indolyl, indazolyl, phosphonyl, phosphatidyl, phosphoramidyl, sulfanyl, sulfinyl, sulfonyl, sulphonamidyl, carbamyl, uryl, thiouryl and thioamidyl, wherein
R⁸ and R¹⁰ taken together with the N atom to which they are attached optionally form an optionally substituted ring comprising 3-7 ring atoms, wherein, in addition to said N atom, 0-3 of the ring atoms are heteroatoms selected from the group consisting of O, S and N;
R⁸ is selected from the group consisting of hydrogen, lower alkyl, lower alkenyl, lower alkynyl, lower heteroalkyl, lower heteroalkenyl, lower heteroalkynyl, lower aryl, lower heteroaryl and —C(O)R⁹;
R⁹ is selected from the group consisting of H, lower alkyl, lower alkenyl, lower alkynyl, lower aryl, lower heteroaryl, —NR¹⁰R¹⁰ and —OR¹¹, wherein
R¹⁰ and R¹⁰ taken together with the N atom to which they are attached optionally form an optionally substituted ring comprising 3-7 ring atoms, wherein, in addition to said N atom, 0-3 of the ring atoms are heteroatoms selected from the group consisting of O, S and N;
R¹⁰ is selected from the group consisting of hydrogen, lower alkyl, lower alkenyl, lower alkynyl, lower heteroalkyl, lower heteroalkenyl, lower heteroalkynyl, lower aryl, lower heteroaryl and —C(O)R¹¹;
R¹¹ is selected from the group consisting of lower alkyl, lower alkenyl, lower alkynyl, lower aryl and lower heteroaryl; and
R¹² is selected from the group consisting of hydrogen and lower alkyl.

34. The method of claim 1 wherein said selected synthetic heterocyclic HSP90 inhibitor is Compounds VII, VIII, IX, XII, XIII and XIV.

35. A method of treating an individual having an HSP90 mediated disorder comprising selecting a synthetic heterocyclic HSP 90 inhibitor wherein the activity of the HSP90 inhibitor is substantially independent of multi drug resistance, and administering to said individual a pharmaceutical composition comprising a pharmaceutically effective amount of said synthetic heterocyclic HSP90 inhibitor, wherein said selected synthetic heterocyclic HSP90 inhibitor has Formula I, or a polymorph, solvate, ester, tautomer, enantiomer, pharmaceutically acceptable salt or prodrug thereof: 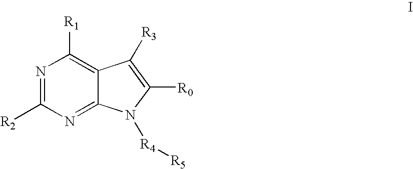
wherein:
R⁰ is selected from hydrogen, halogen, lower alkyl, —SR⁸, —OR⁸, —CN, and —NHR⁸,
R¹ is halogen, —OR¹¹, —SR¹¹ or lower alkyl;
R² is —NHR⁸;
R³ is selected from the group consisting of hydrogen, halogen, —SR⁸, —OR⁸, —CN, —C(O)R⁹, —C(O)OH, —NO₂, —NR⁸R¹⁰, lower alkyl, lower alkenyl, lower alkynyl, lower perhaloalkyl, aryl, heteroaryl, alicyclic and heterocyclic, all optionally substituted, wherein:
the aryl, heteroaryl, alicyclic and heterocyclic groups are optionally mono-, bi- or tri-cyclic,
R⁸ and R¹⁰ taken together optionally form a ring of 3-7 ring atoms and optionally 1-3 of the ring atoms are heteroatoms selected from the group of O, S and N, and
the optional substituents on R³ are selected from the group consisting of halogen, lower alkyl, lower alkenyl, lower alkynyl, —SR⁸, —OR⁸, —CN, —C(O)OH, —C(O)R⁹, —NO₂, —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;
R⁰ or R³ is —OH or —SH, the compound may exist as the corresponding (thio)keto tautomer or a mixture of keto-enol tautomers;
R⁴ is —CHR¹²—;
R⁵ is aryl, heteroaryl, alicyclic, or heterocyclic, wherein
the aryl group is substituted with 3 to 5 substituents,
the heteroaryl group is substituted with 2 to 5 substituents,
the alicyclic group is substituted with 3 to 5 substituents,
the heterocyclic group is substituted with 3 to 5 substituents, and
the substituents are selected from the group consisting of halogen, lower alkyl, lower alkenyl, lower alkynyl, —SR⁸, —OR⁸, —CN, —C(O)OH, —C(O)R⁹, —NO₂, —NR⁸R¹⁰, lower aryl, heteroaryl, alicyclic, lower heterocyclic, arylalkyl, heteroarylalkyl, amino, alkylamino, dialkylamino, diarylalkylamino, oxo, oxa, perhaloalkyl, perhaloalkoxy, perhaloacyl, guanidine, pyridinyl, thiophene, furanyl, indole, indazole, phosphonates, phosphates, phosphoramides, sulfonates, sulfones, sulfates, sulphonamides, carbamates, ureas, thioureas and thioamides, wherein R⁸ and R¹⁰ taken together optionally form a ring of 3-7 ring atoms and optionally 1-3 of the ring atoms are heteroatoms selected from the group of O, S and N;
R⁸ is hydrogen, lower alkyl, lower alkenyl, or lower alkynyl, lower aryl, lower heteroaryl, or —C(O)R⁹;
R⁹ is H, lower alkyl, lower alkenyl, lower alkynyl, lower aryl, lower heteroaryl, —NR¹⁰R¹⁰, or —OR¹¹, wherein R¹⁰ and R¹⁰ taken together optionally form a ring of 3-7 ring atoms and optionally 1-3 of the ring atoms are heteroatoms selected from the group of O, S and N;
R¹⁰ is hydrogen, lower alkyl, lower alkenyl, lower alkynyl, lower aryl or lower heteroaryl;
R¹¹ is lower alkyl, lower alkenyl, lower alkynyl, lower heteroaryl or lower aryl; and
R¹² is hydrogen or lower alkyl.

36. A method of treating an individual having an HSP90 mediated disorder comprising selecting a synthetic heterocyclic HSP 90 inhibitor wherein the activity of the HSP90 inhibitor is substantially independent of multi drug resistance, and administering to said individual a pharmaceutical composition comprising a pharmaceutically effective amount of said synthetic heterocyclic HSP90 inhibitor, wherein said selected synthetic heterocyclic HSP90 inhibitor is selected from the group below, or a polymorph, solvate, ester, tautomer, enantiomer, pharmaceutically acceptable salt or prodrug thereof:

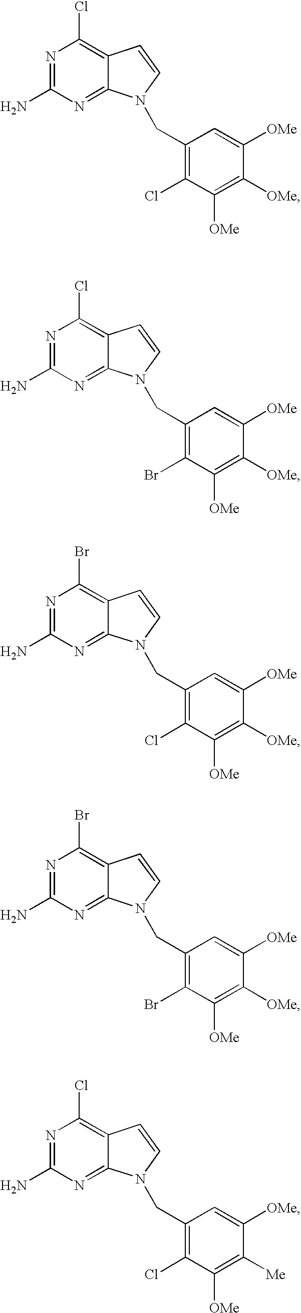

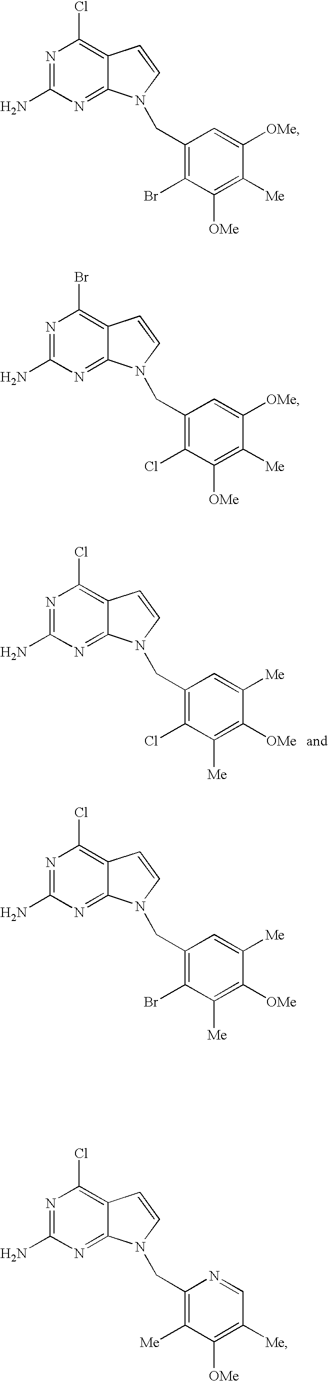

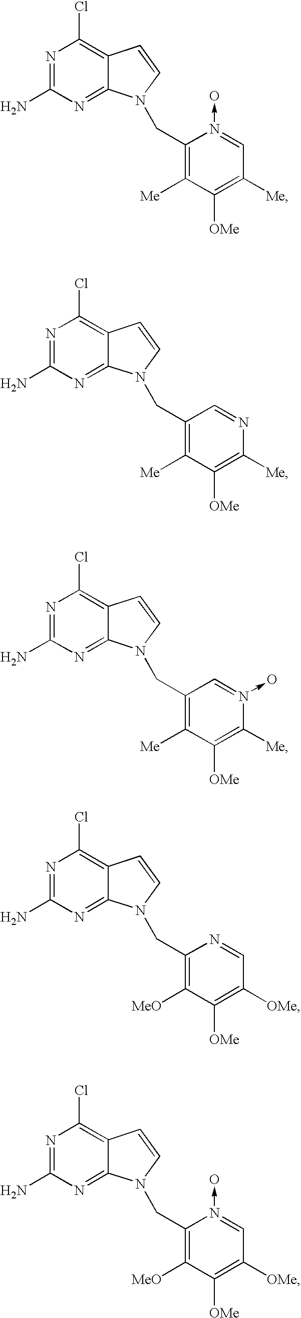

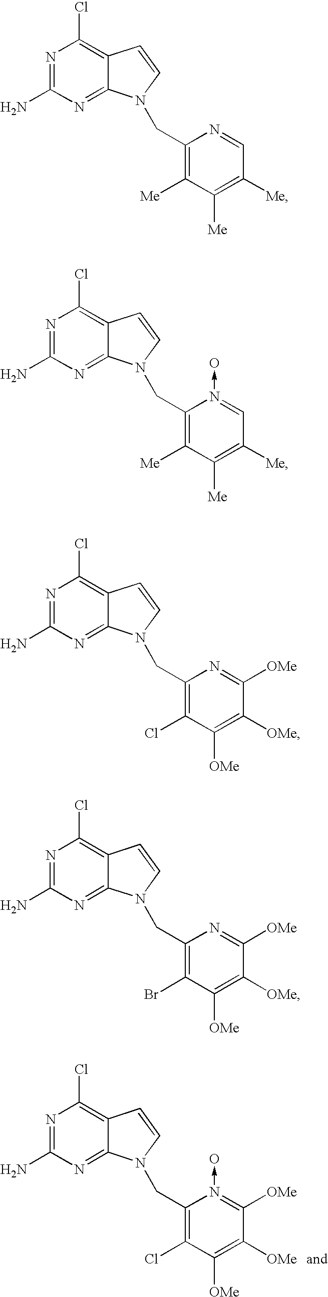

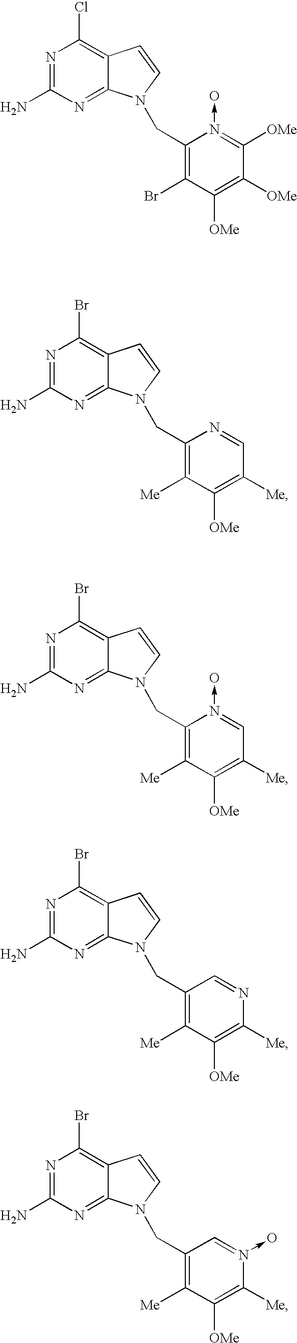

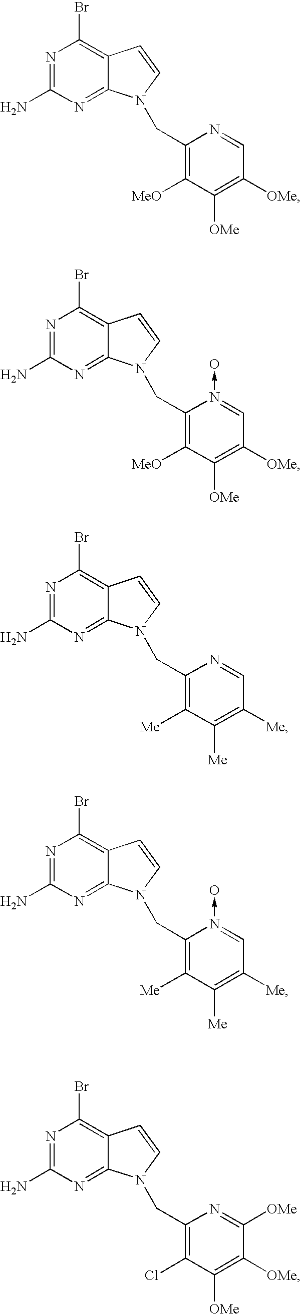

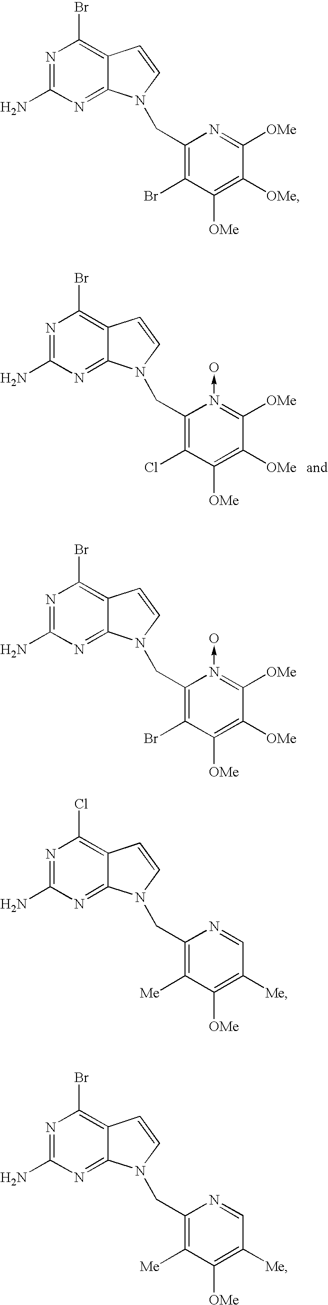

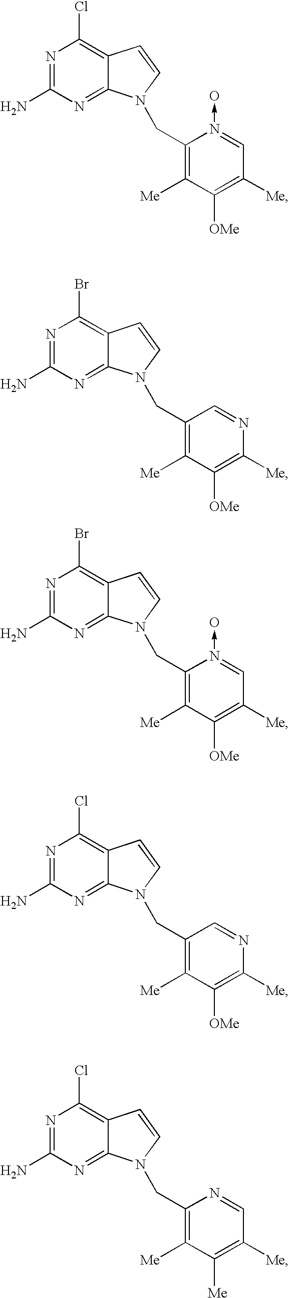

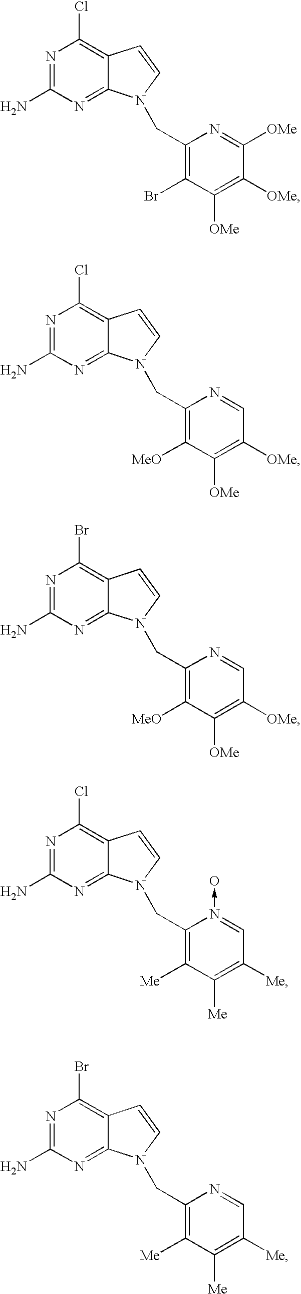

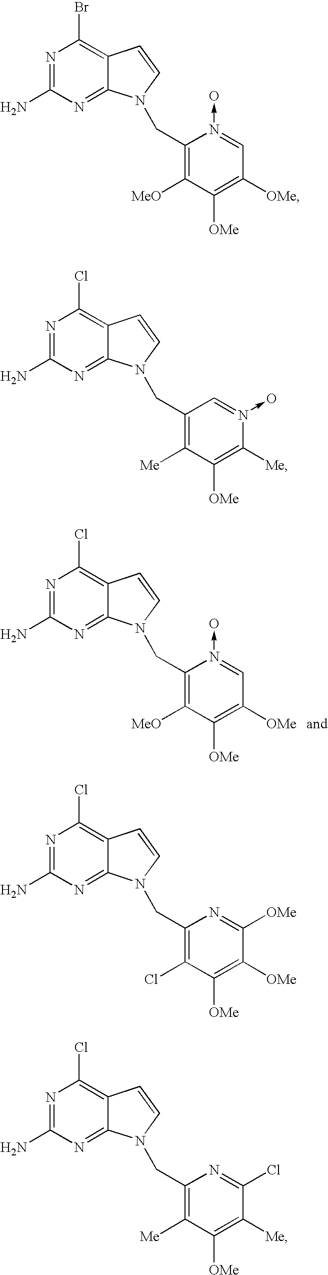

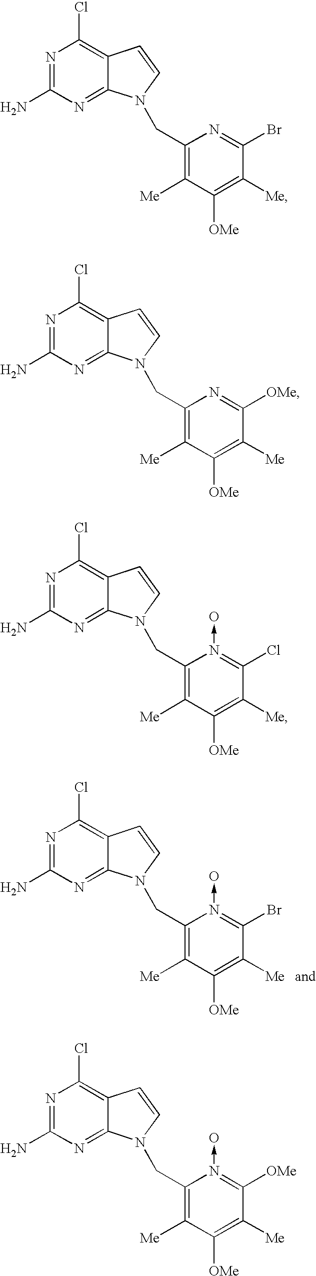

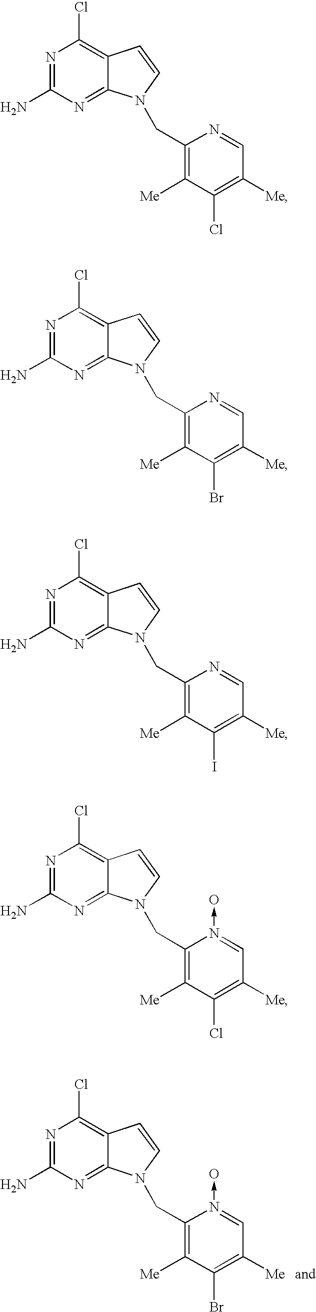

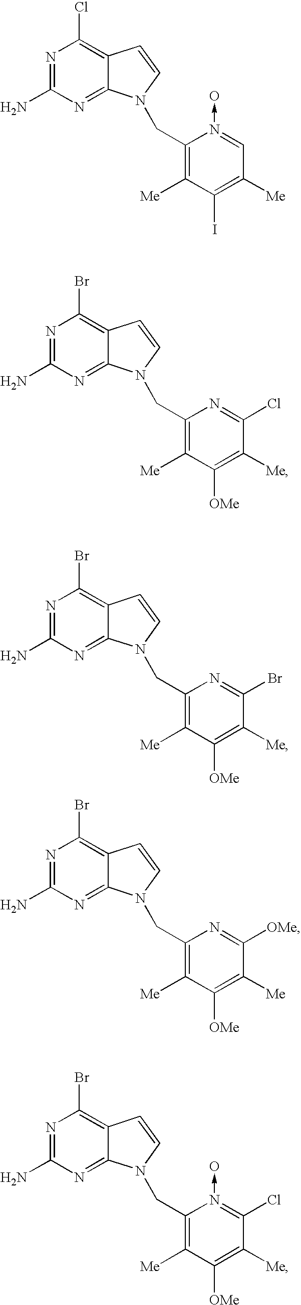

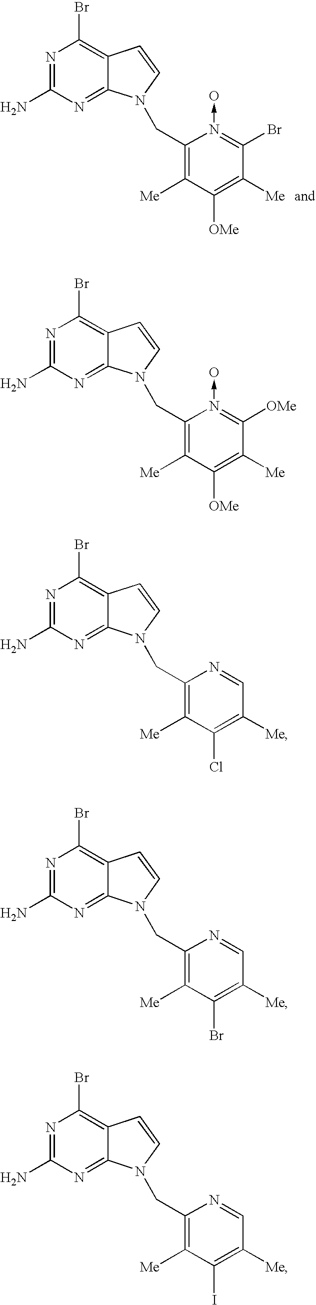

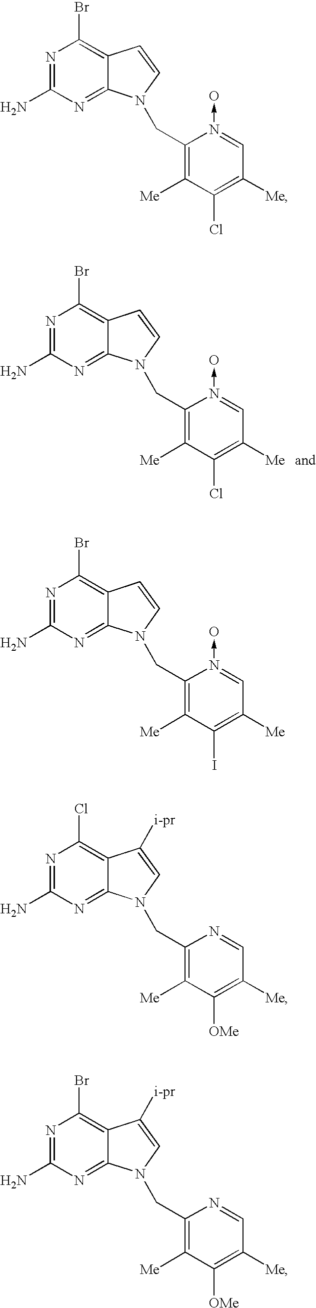

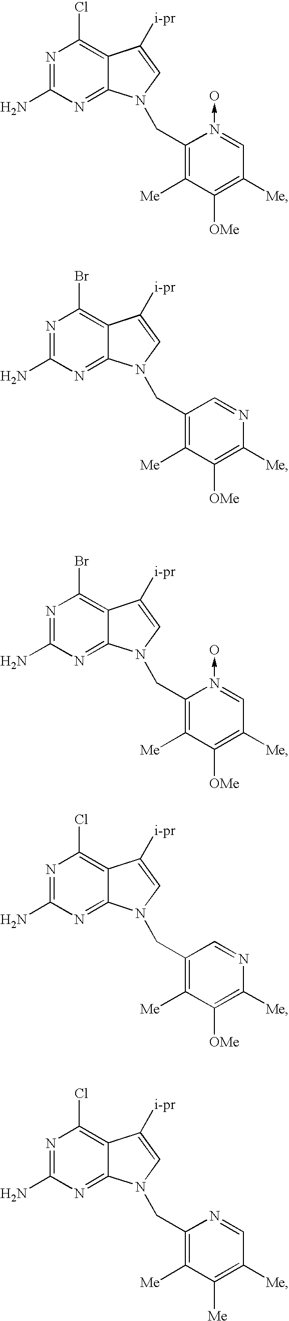

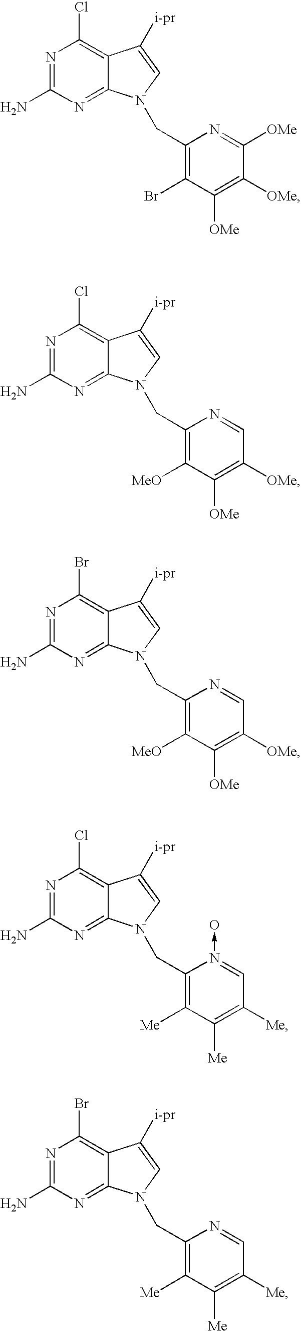

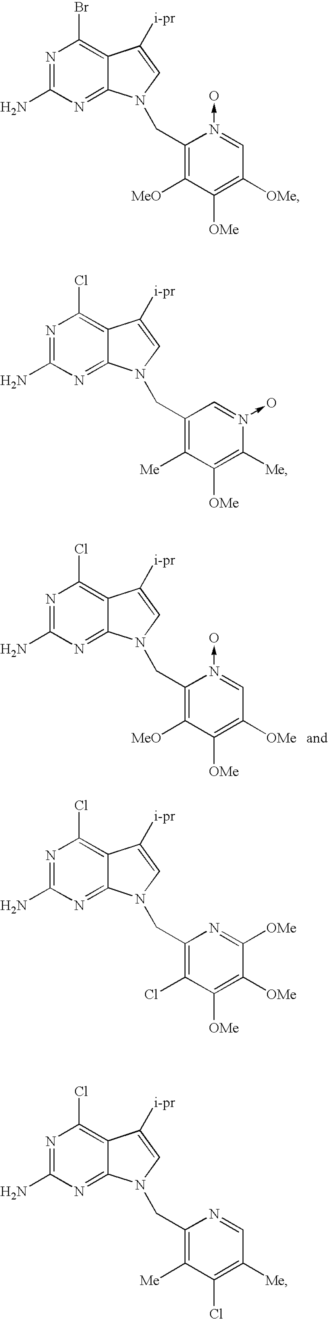

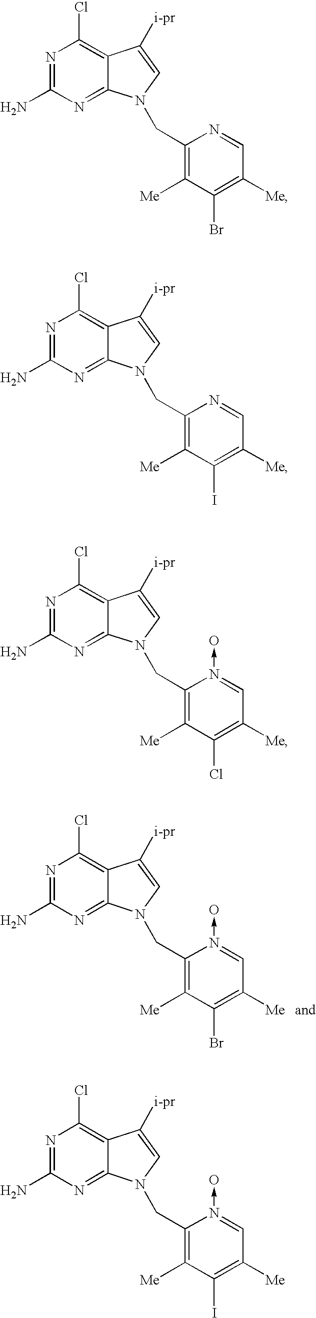

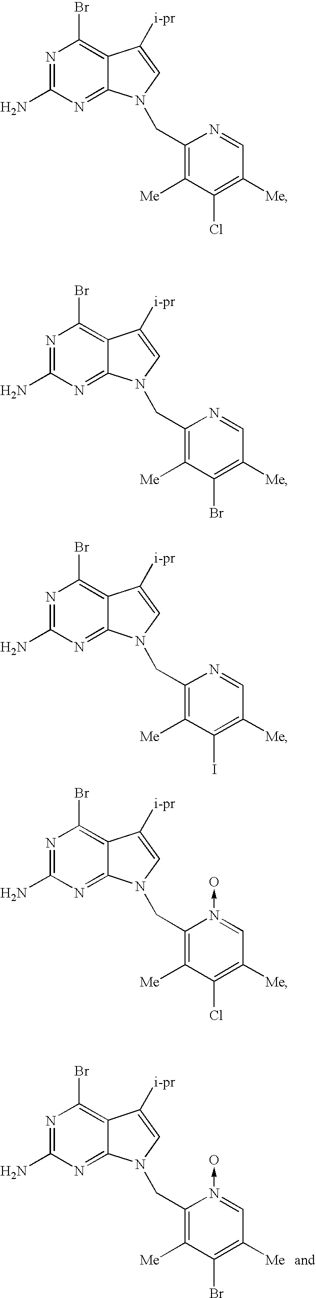

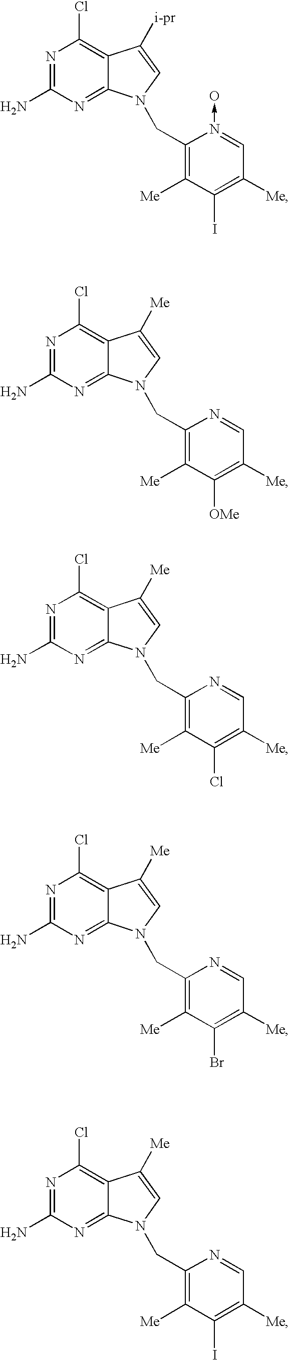

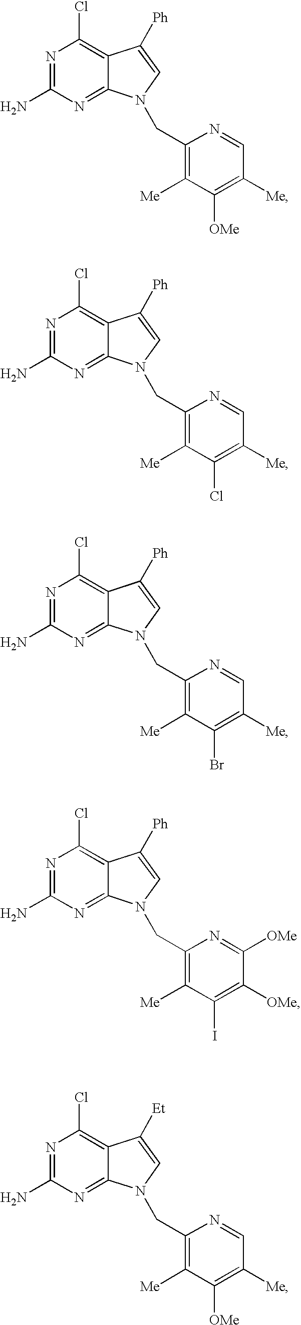

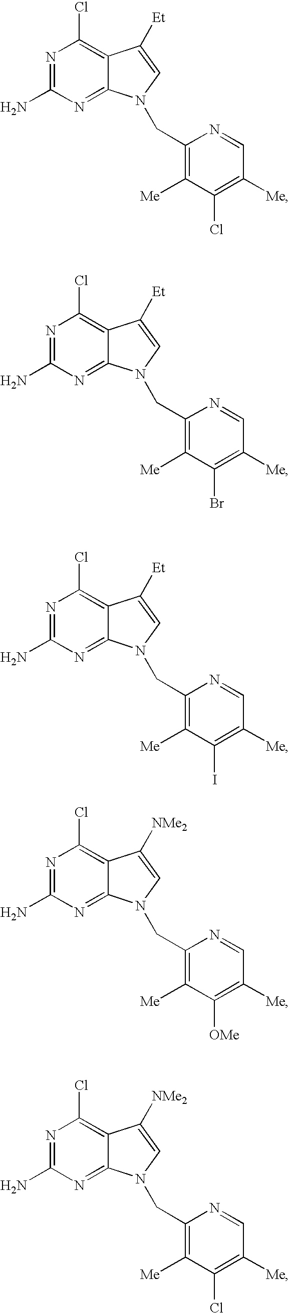

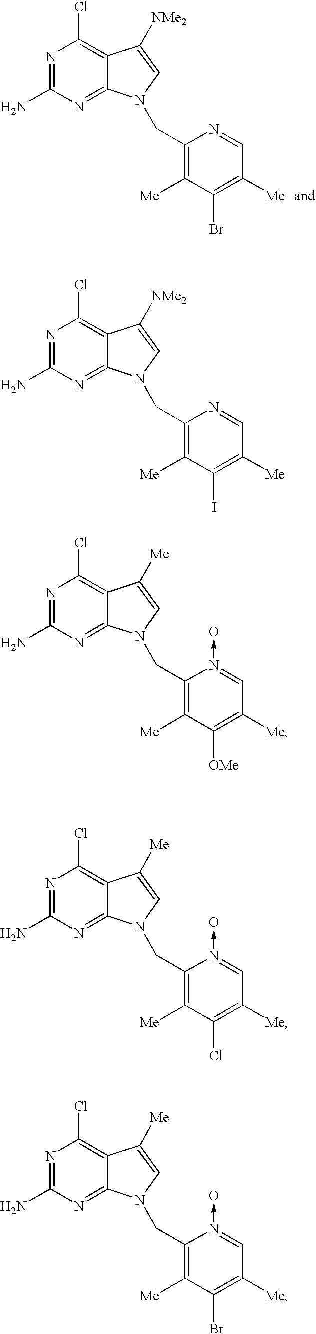

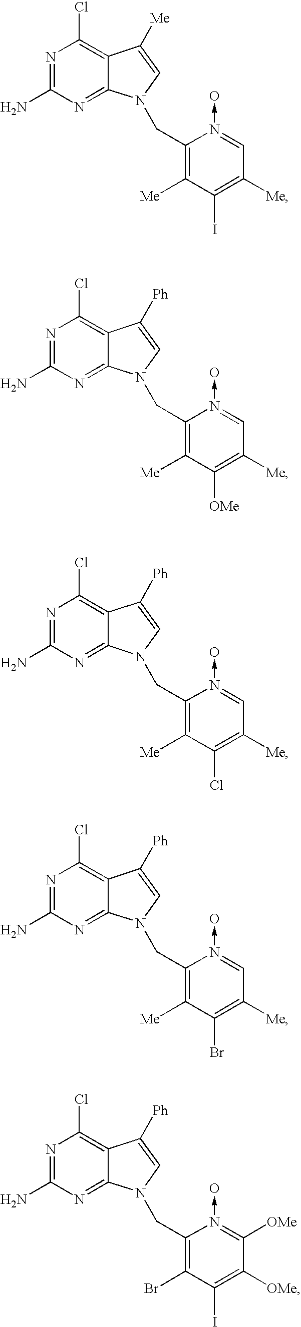

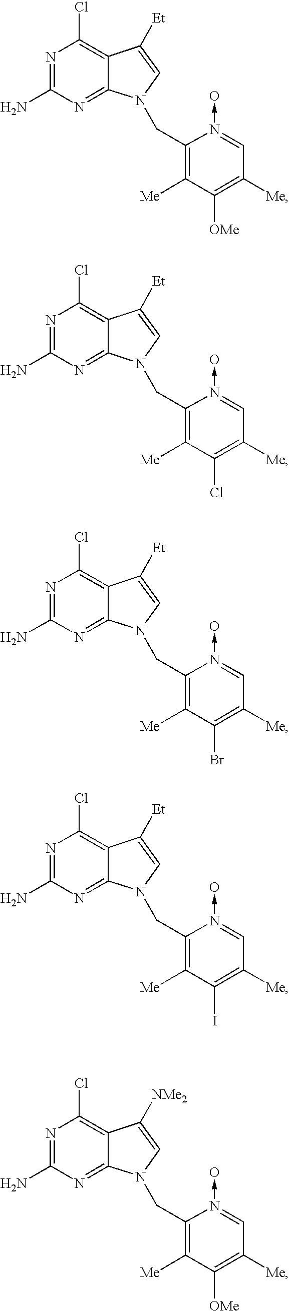

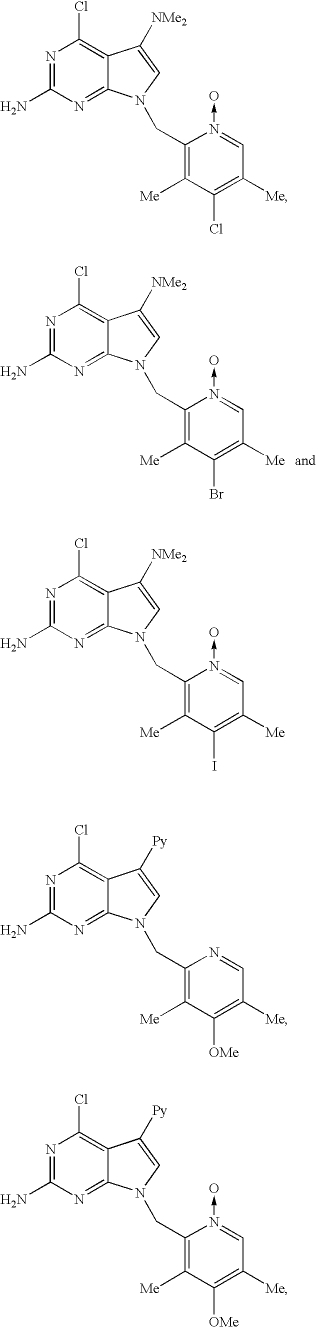

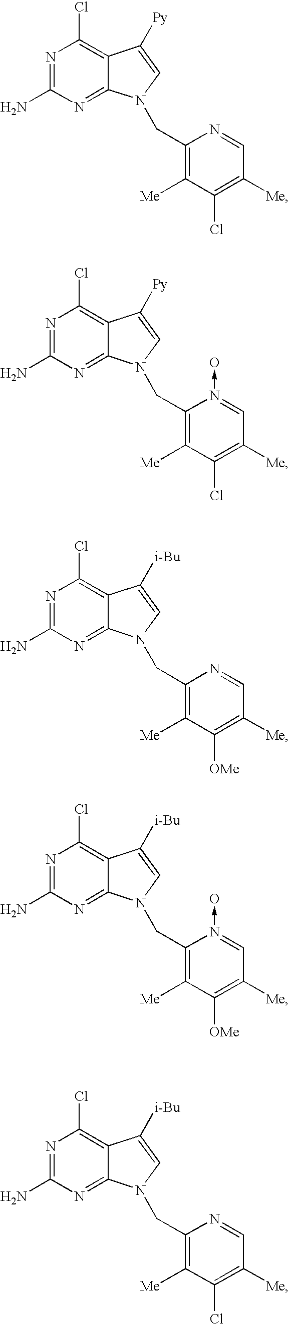

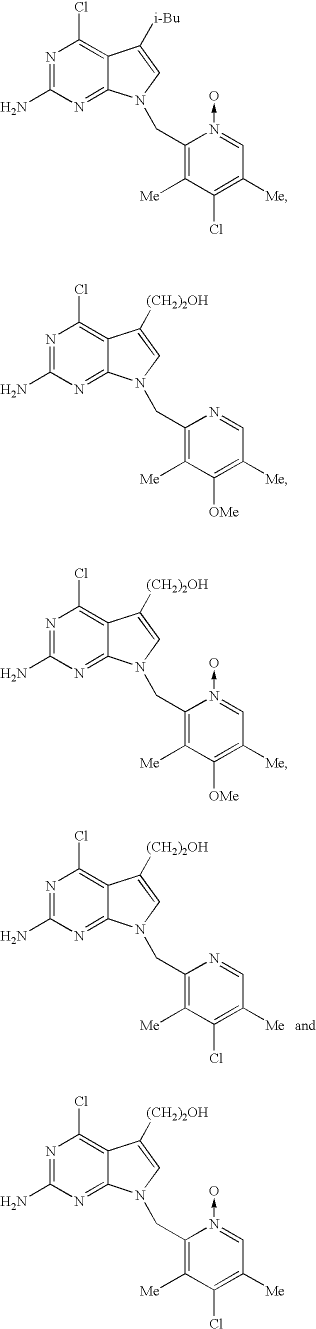

37. A method of treating an individual having an HSP90 mediated disorder comprising selecting a synthetic heterocyclic HSP 90 inhibitor wherein the activity of the HSP90 inhibitor is substantially independent of multi drug resistance, and administering to said individual a pharmaceutical composition comprising a pharmaceutically effective amount of said synthetic heterocyclic HSP90 inhibitor, wherein said selected synthetic heterocyclic HSP90 inhibitor is selected from the group below, or a polymorph, solvate, ester, tautomer, enantiomers, pharmaceutically acceptable salt or prodrug thereof:

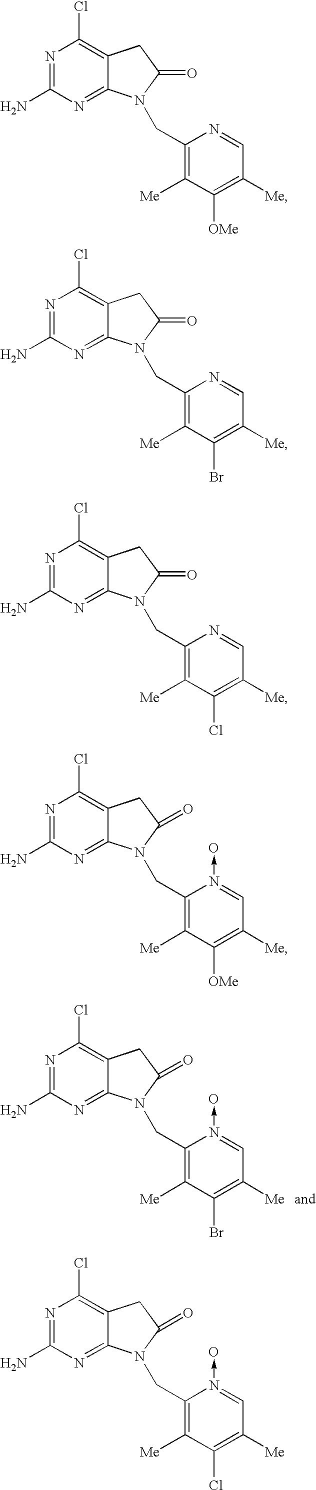

38. A method of treating an individual having an HSP90 mediated disorder comprising selecting a synthetic heterocyclic HSP 90 inhibitor wherein the activity of the HSP90 inhibitor is substantially independent of multi drug resistance, and administering to said individual a pharmaceutical composition comprising a pharmaceutically effective amount of said synthetic heterocyclic HSP90 inhibitor, wherein the activity of the HSP90 inhibitor is substantially independent of multi drug resistance and said selected synthetic heterocyclic HSP90 inhibitor is selected from the group below, or a polymorph, solvate, ester, tautomer, enantiomers, pharmaceutically acceptable salt or prodrug thereof:

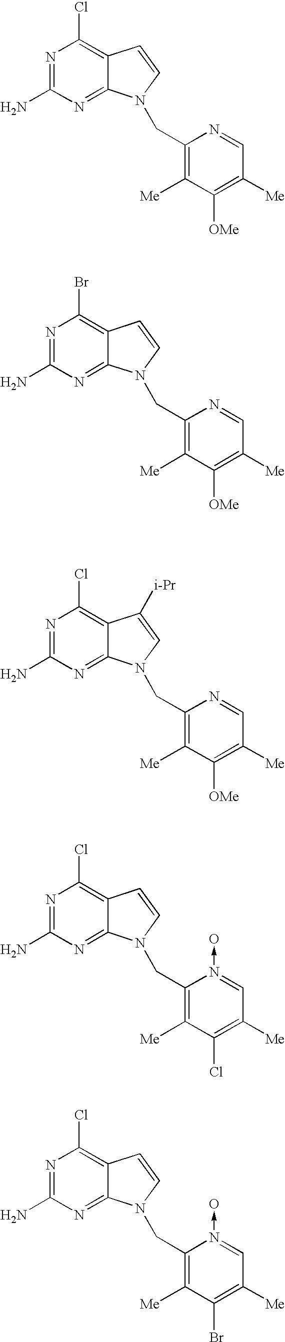

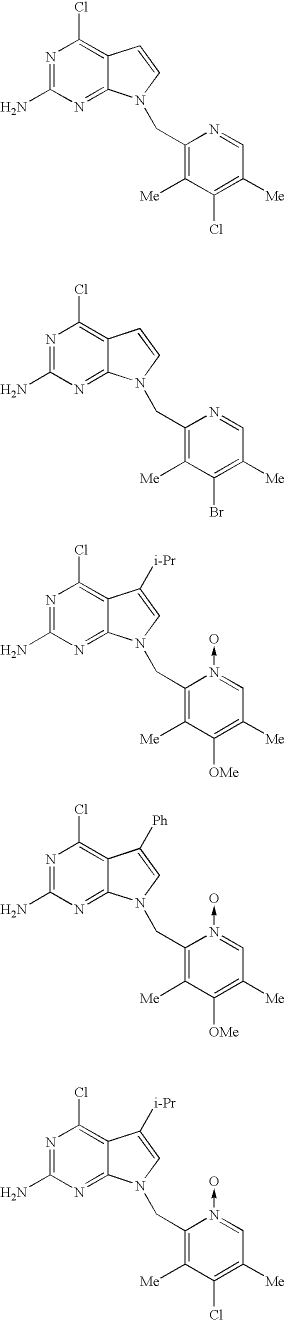

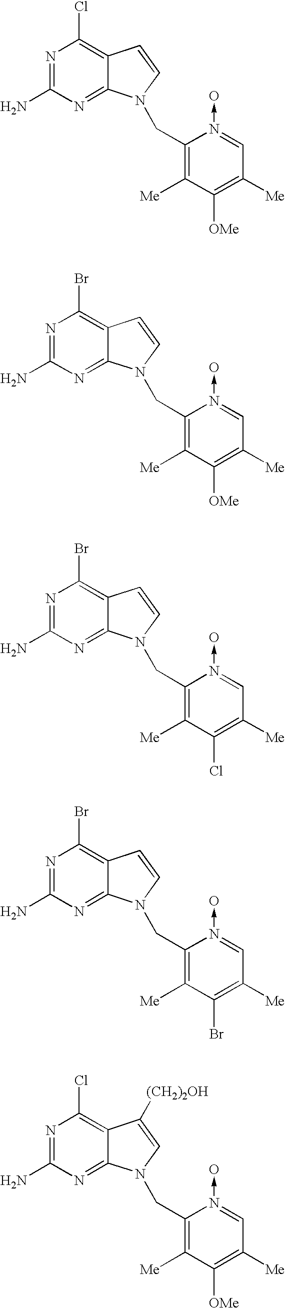

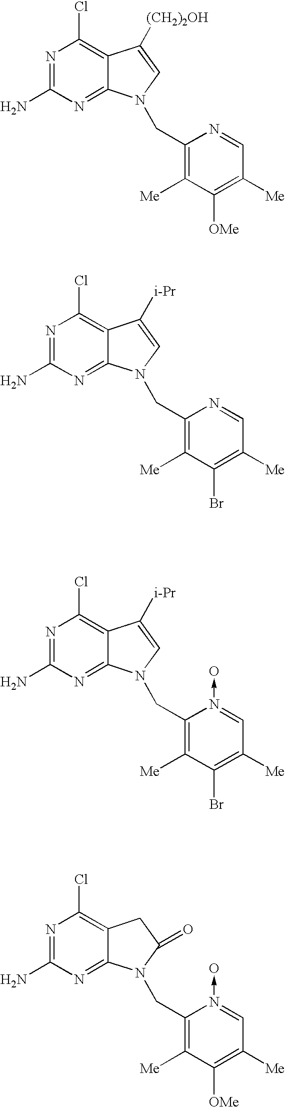

39. A method of treating an individual having an HSP90 mediated disorder comprising selecting a synthetic heterocyclic HSP 90 inhibitor wherein the activity of the HSP90 inhibitor is substantially independent of multi drug resistance, and administering to said individual a pharmaceutical composition comprising a pharmaceutically effective amount of said synthetic heterocyclic HSP90 inhibitor, wherein said selected synthetic heterocyclic HSP90 inhibitor is selected from the group below, or a polymorph, solvate, ester, tautomer, enantiomers, pharmaceutically acceptable salt or prodrug thereof:

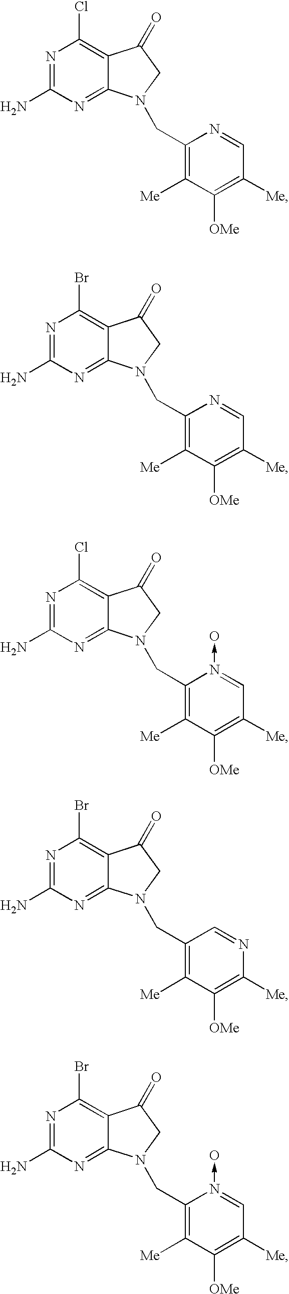

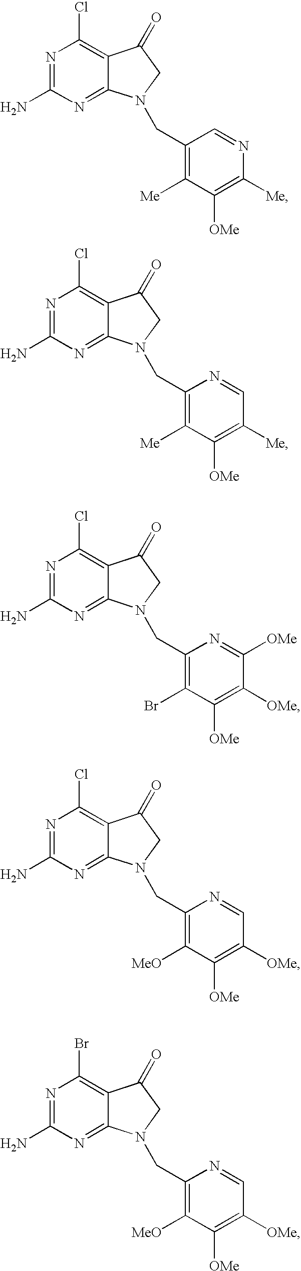

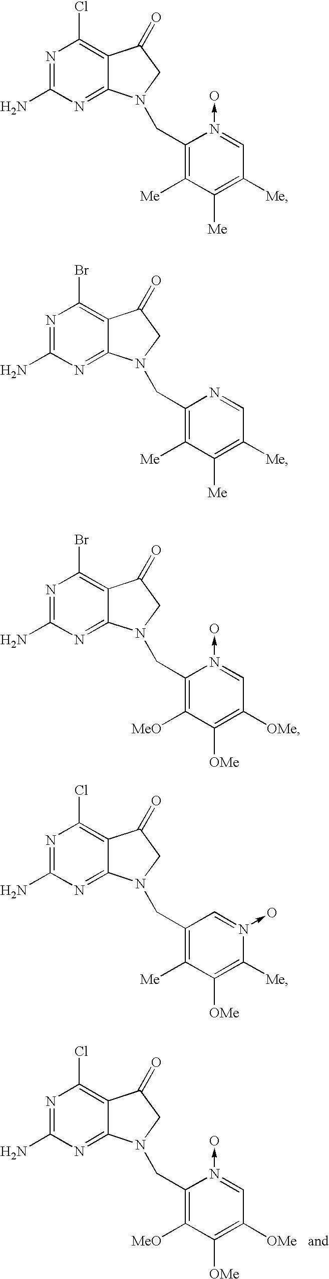

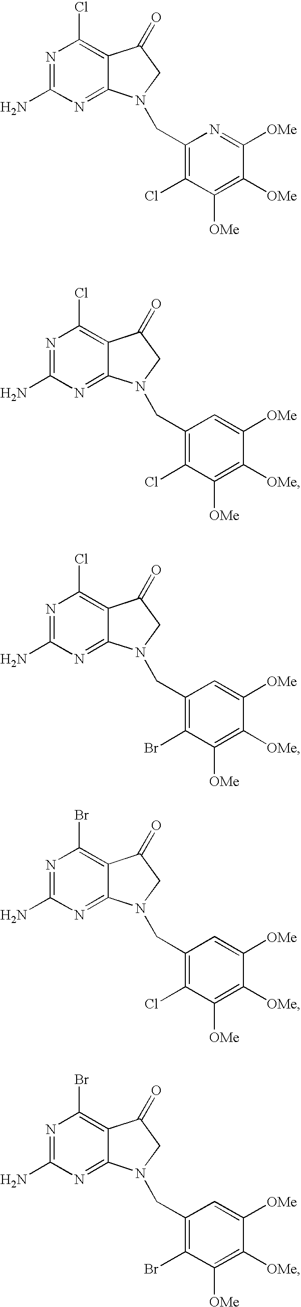

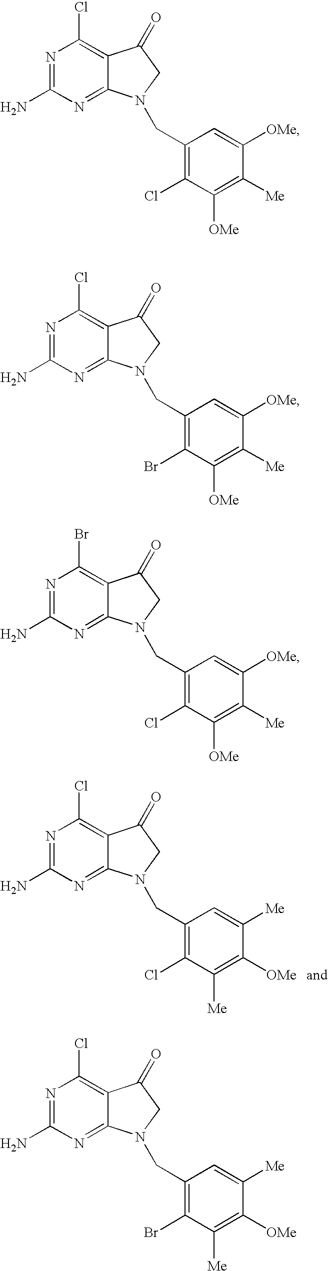

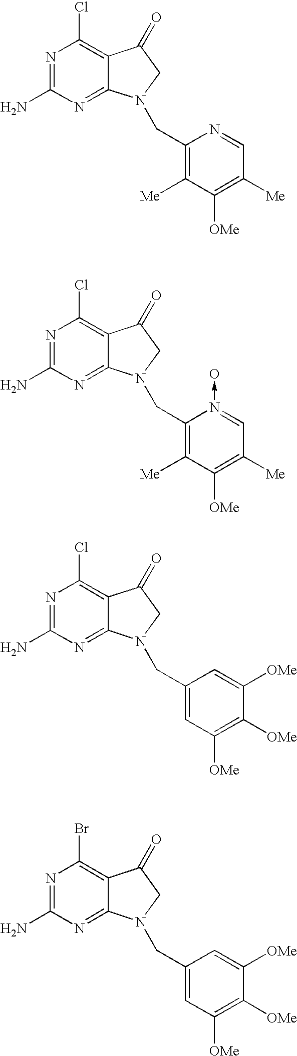

40. A method of treating an individual having an HSP90 mediated disorder comprising selecting a synthetic heterocyclic HSP 90 inhibitor wherein the activity of the HSP90 inhibitor is substantially independent of multi drug resistance, and administering to said individual a pharmaceutical composition comprising a pharmaceutically effective amount of said synthetic heterocyclic HSP90 inhibitor, wherein said selected synthetic heterocyclic HSP90 inhibitor is selected from the group below, or a polymorph, solvate, ester, tautomer, enantiomers, pharmaceutically acceptable salt or prodrug thereof:

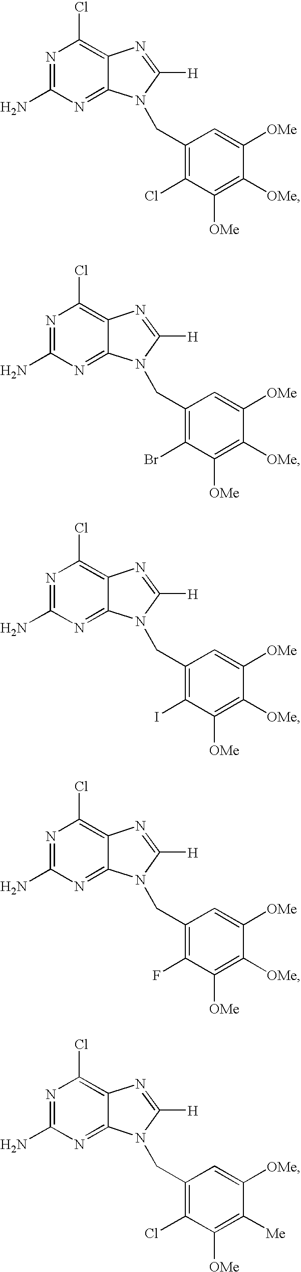

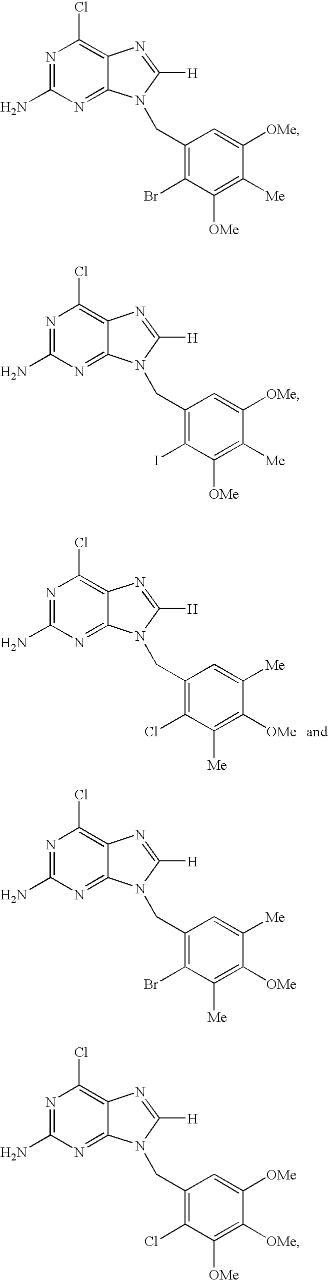

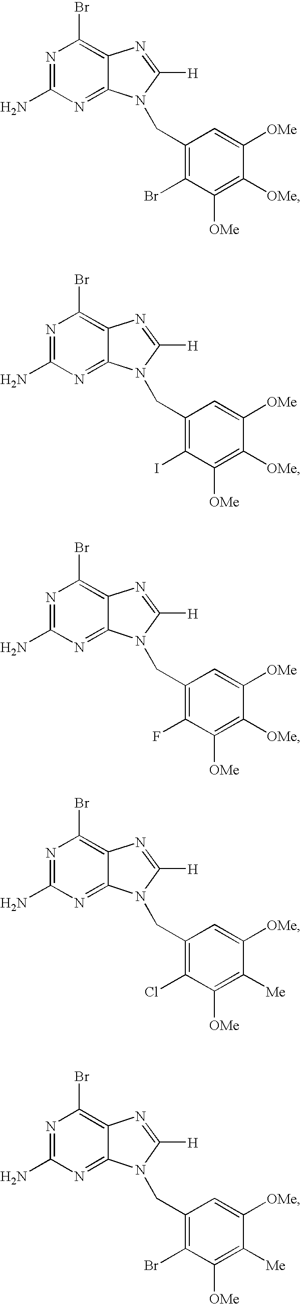

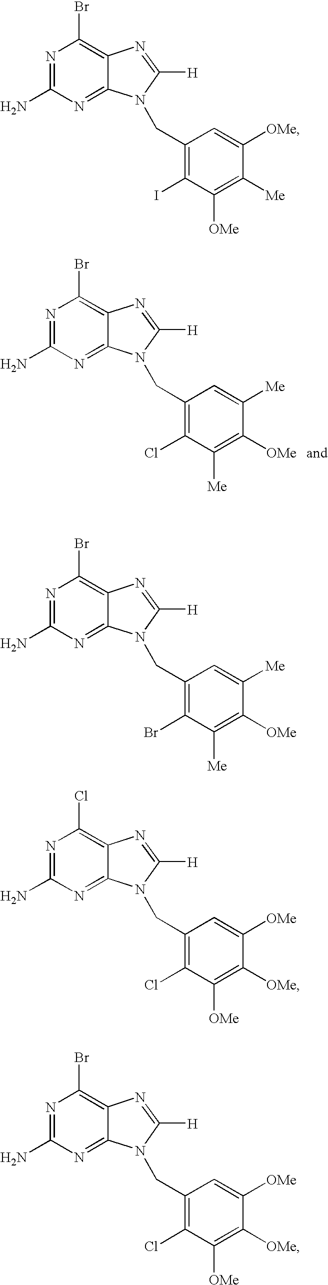

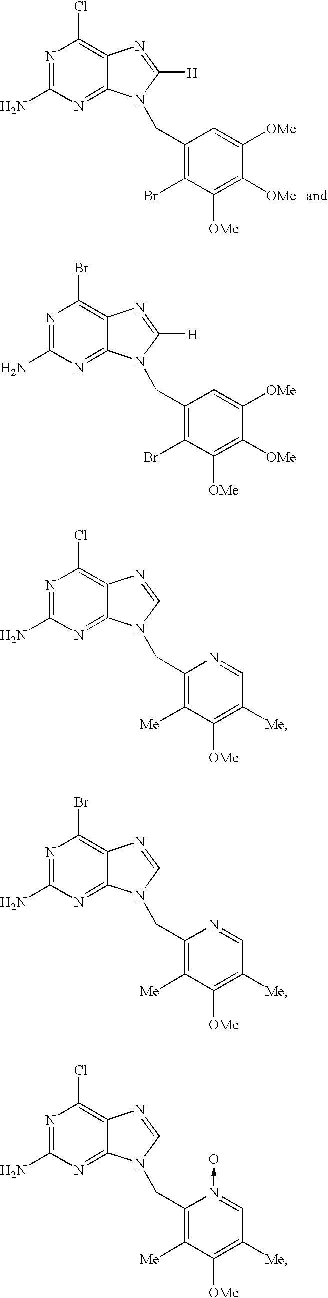

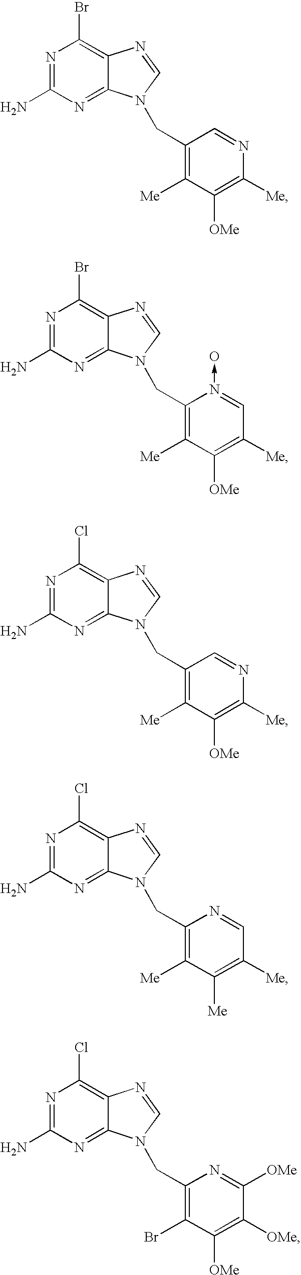

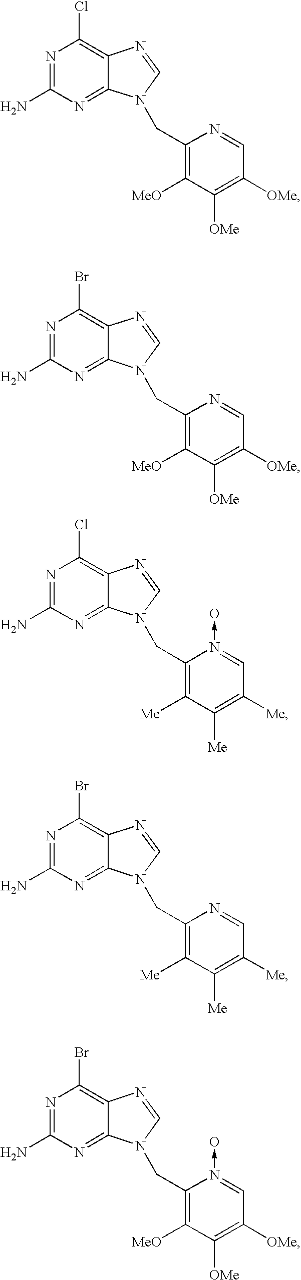

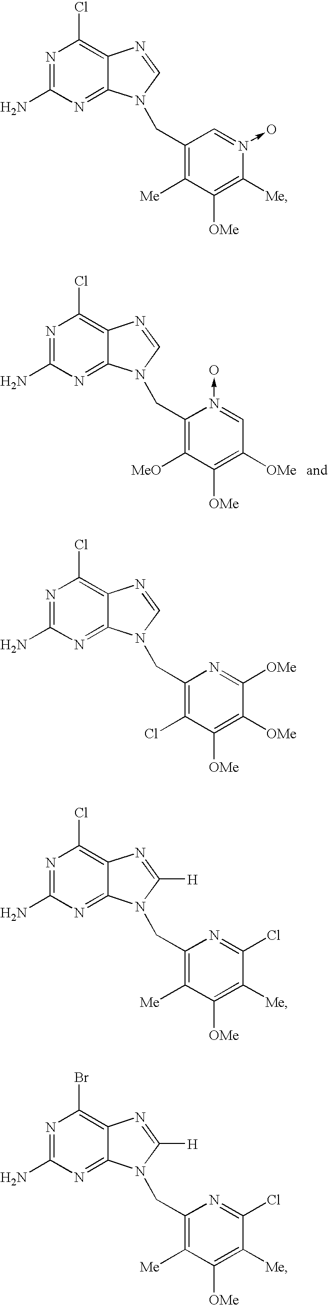

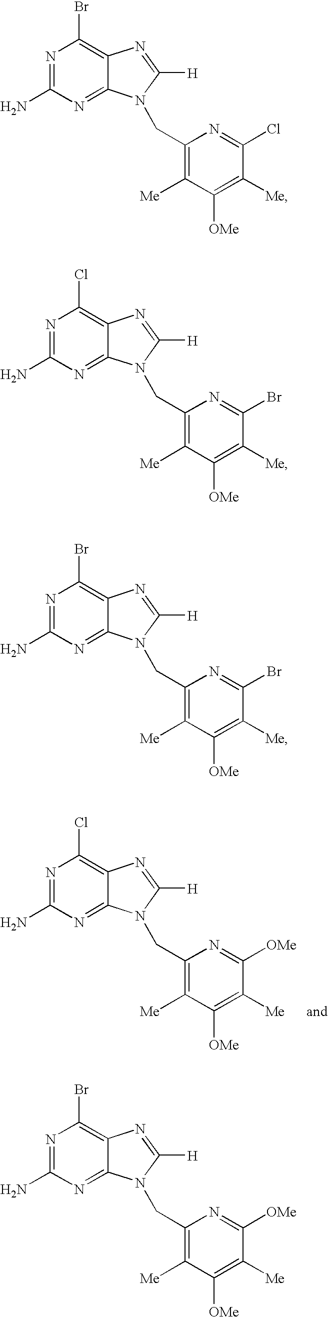

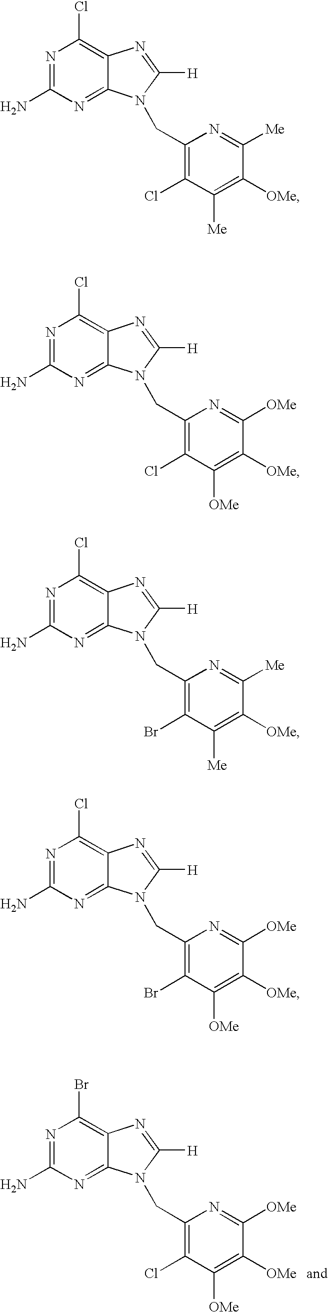

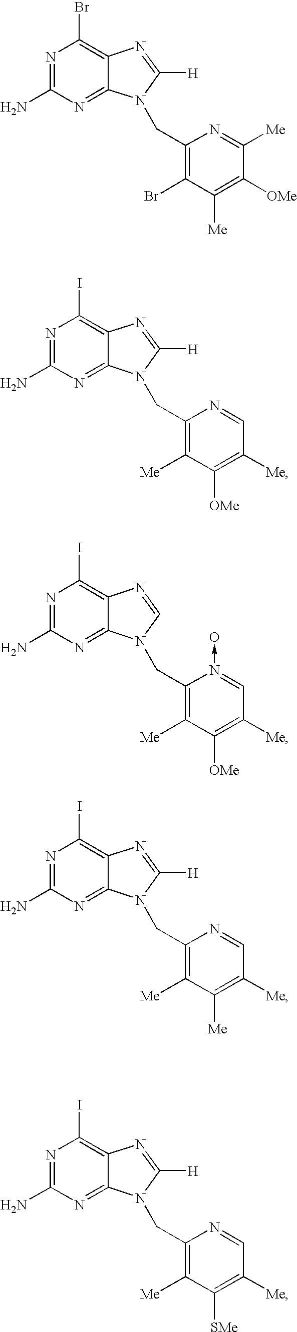

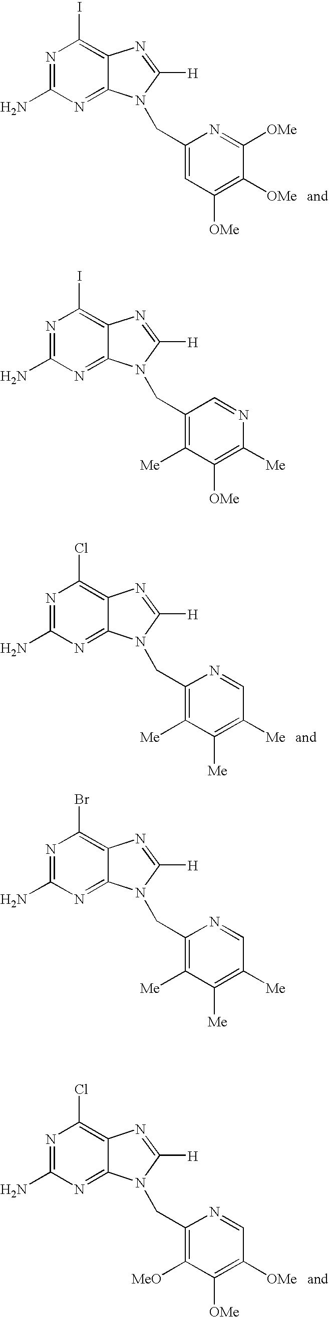

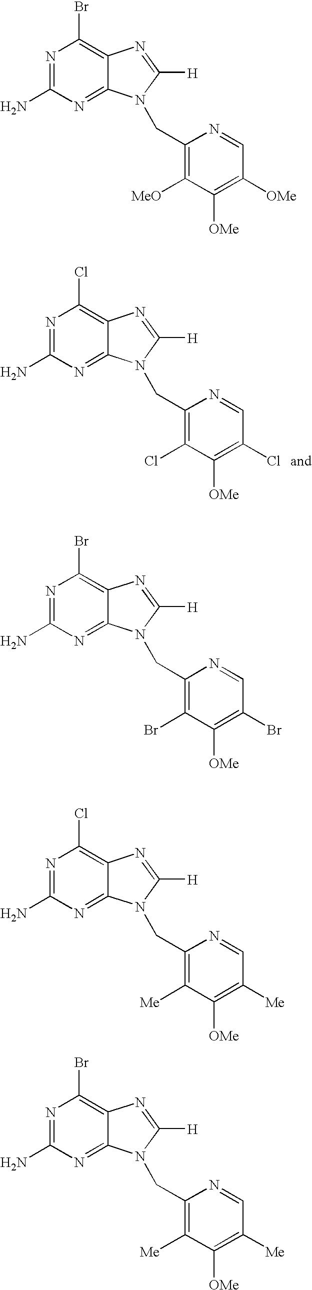

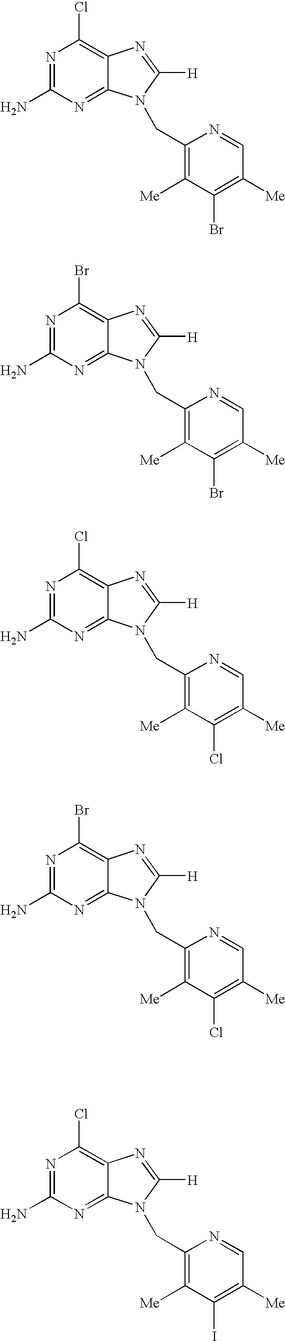

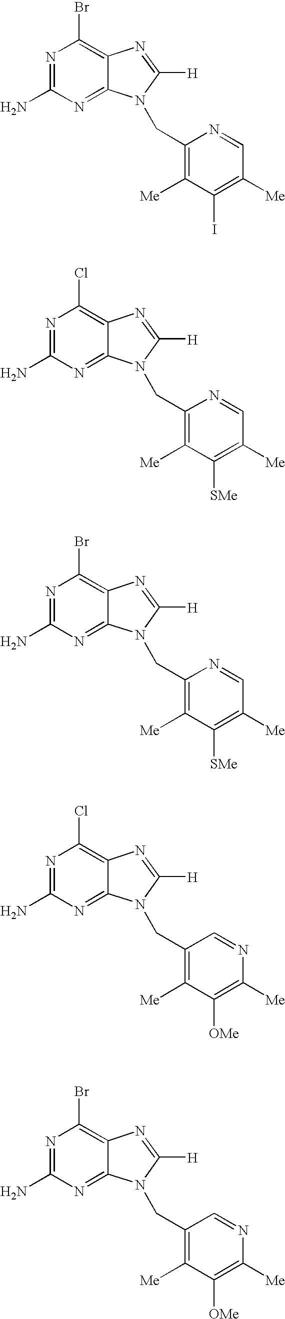

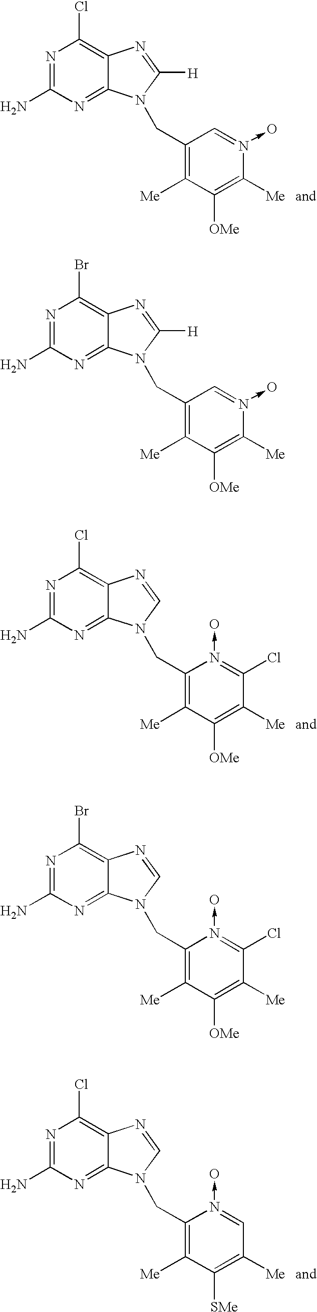

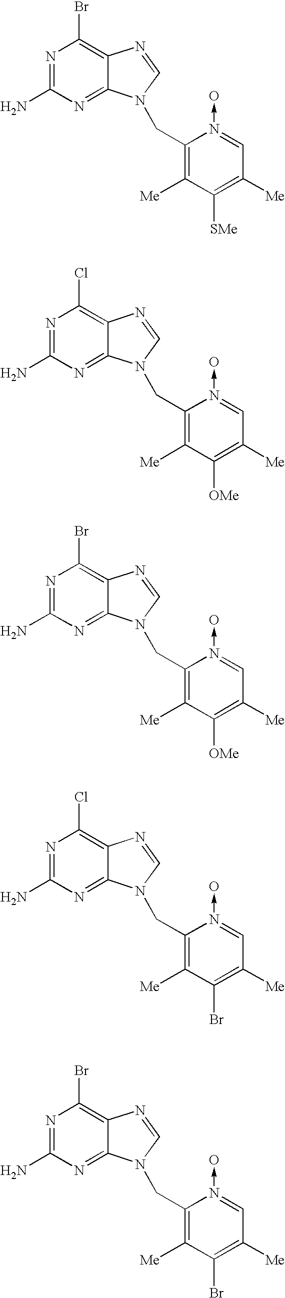

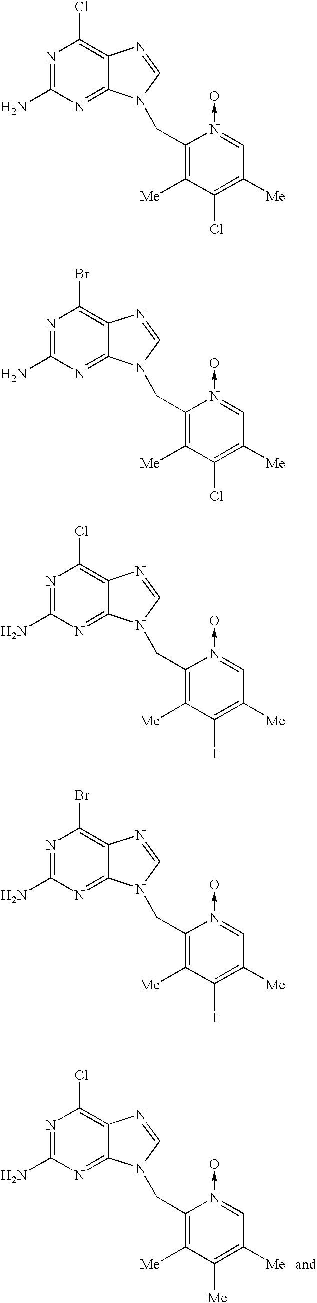

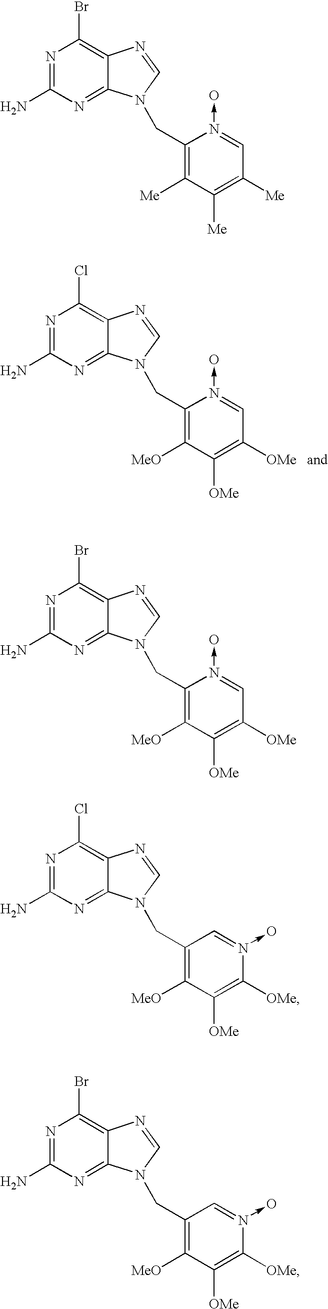

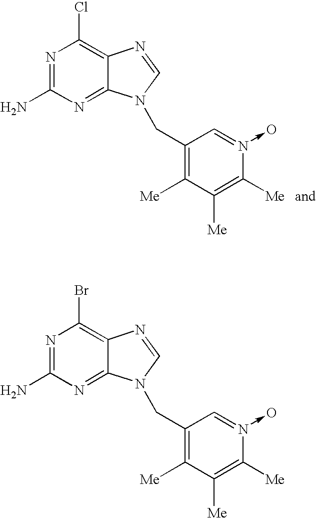

41. A method of treating an individual having an HSP90 mediated disorder comprising selecting a synthetic heterocyclic HSP 90 inhibitor wherein the activity of the HSP90 inhibitor is substantially independent of multi drug resistance, and administering to said individual a pharmaceutical composition comprising a pharmaceutically effective amount of said synthetic heterocyclic HSP90 inhibitor, wherein said selected synthetic heterocyclic HSP90 inhibitor is selected from the group below, or a polymorph, solvate, ester, tautomer, enantiomers, pharmaceutically acceptable salt or prodrug thereof:

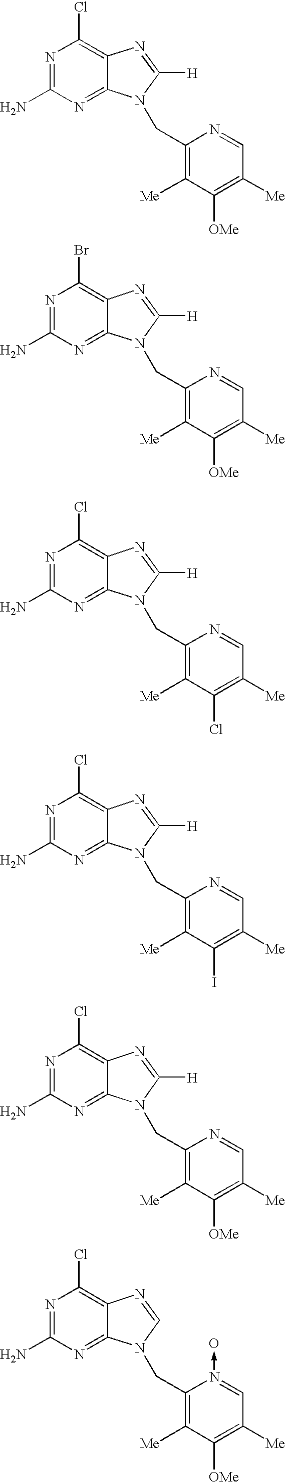

42. A method of treating an individual having an HSP90 mediated disorder comprising selecting a synthetic heterocyclic HSP 90 inhibitor wherein the activity of the HSP90 inhibitor is substantially independent of multi drug resistance, and administering to said individual a pharmaceutical composition comprising a pharmaceutically effective amount of said synthetic heterocyclic HSP90 inhibitor, wherein said selected synthetic heterocyclic HSP90 inhibitor is selected from the group below, or a polymorph, solvate, ester, tautomer, enantiomers, pharmaceutically acceptable salt or prodrug thereof:

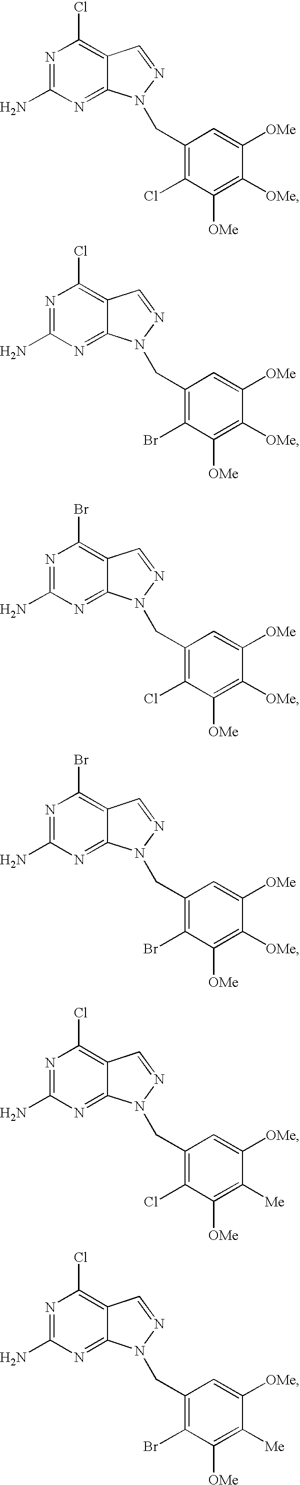

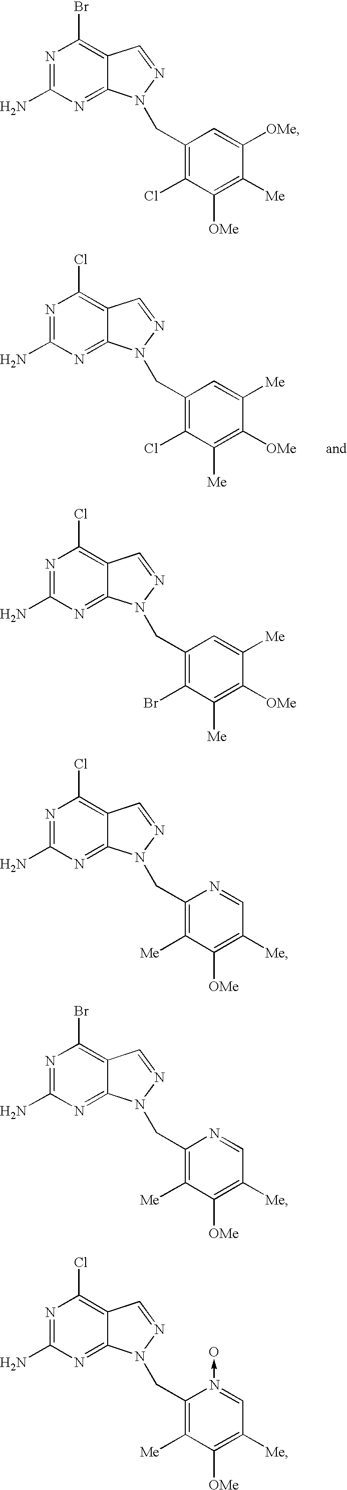

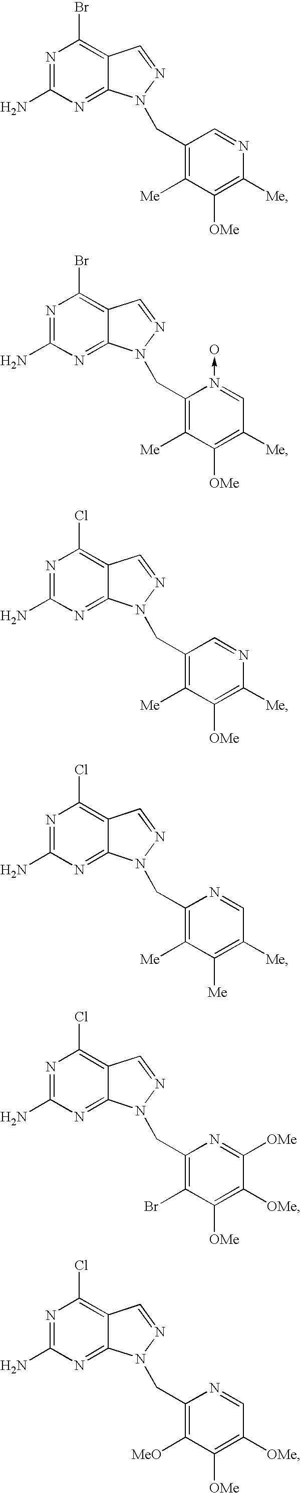

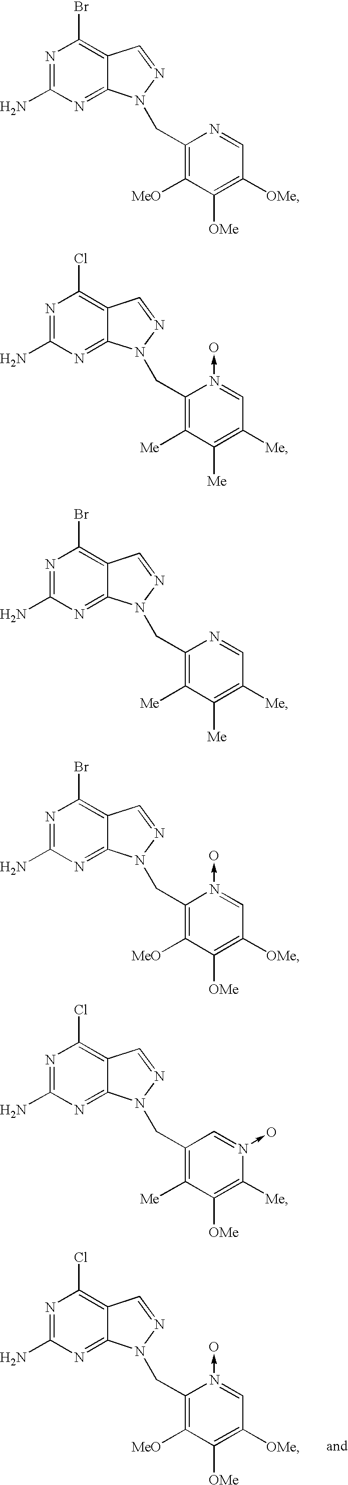

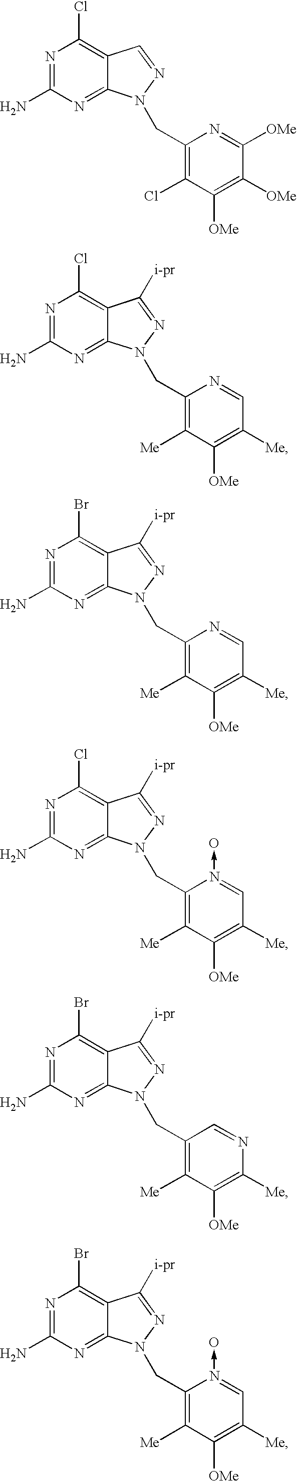

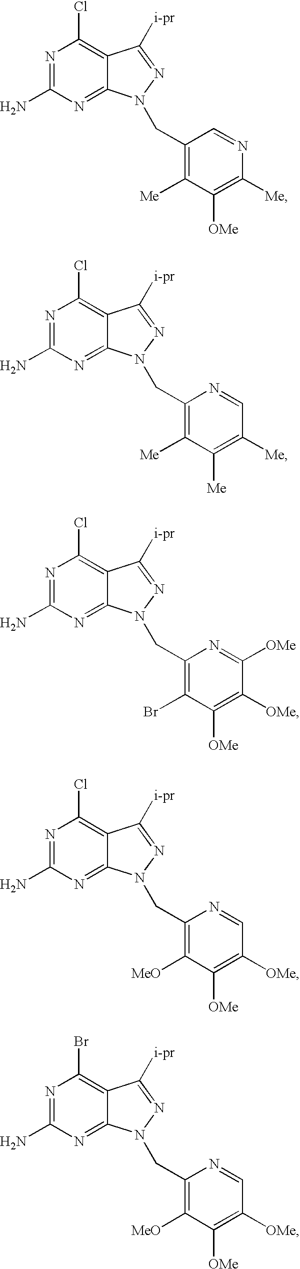

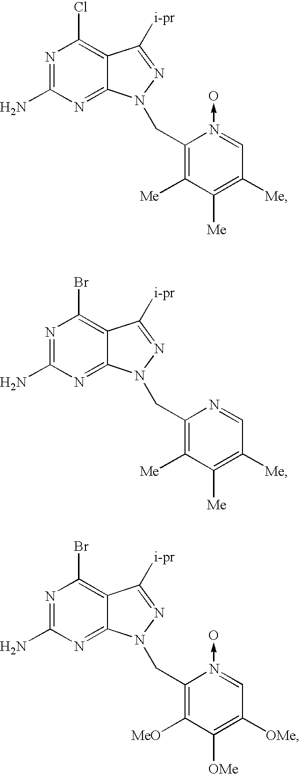

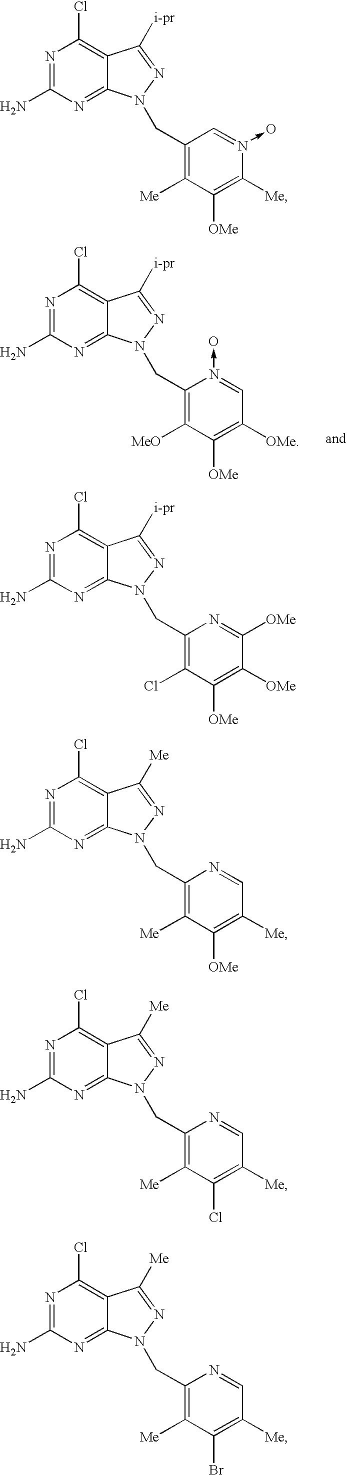

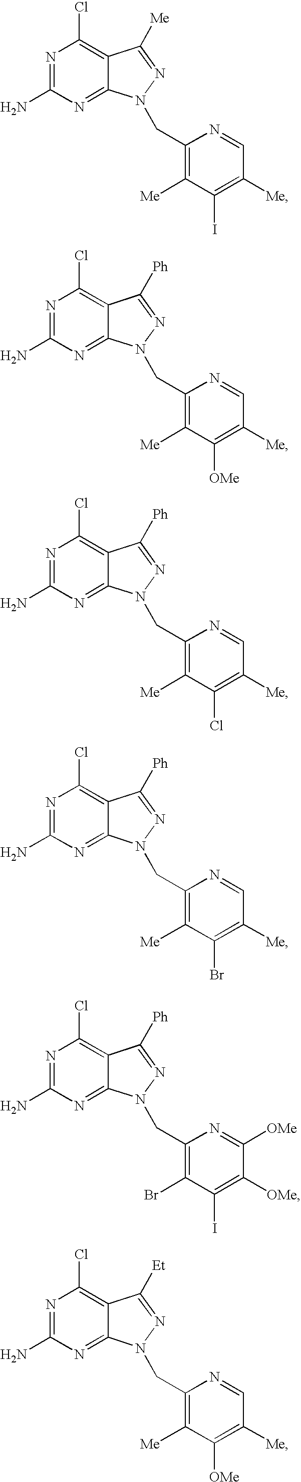

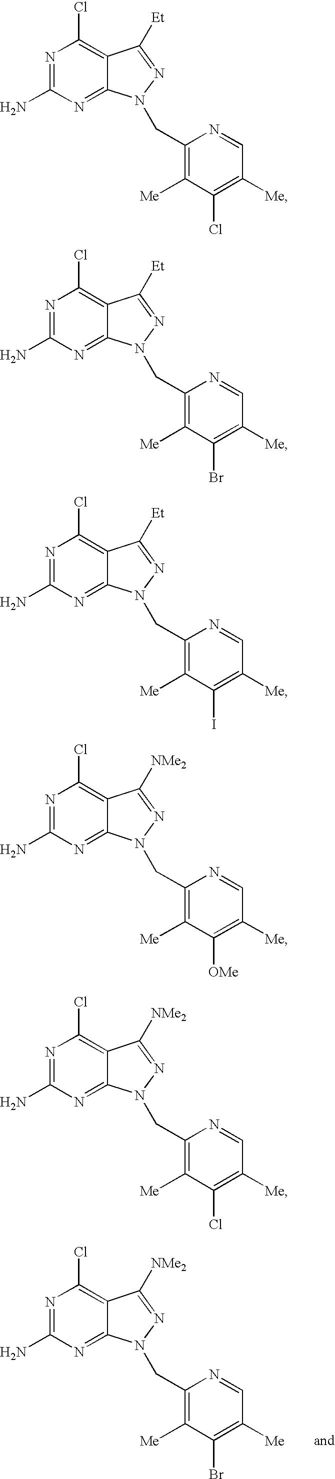

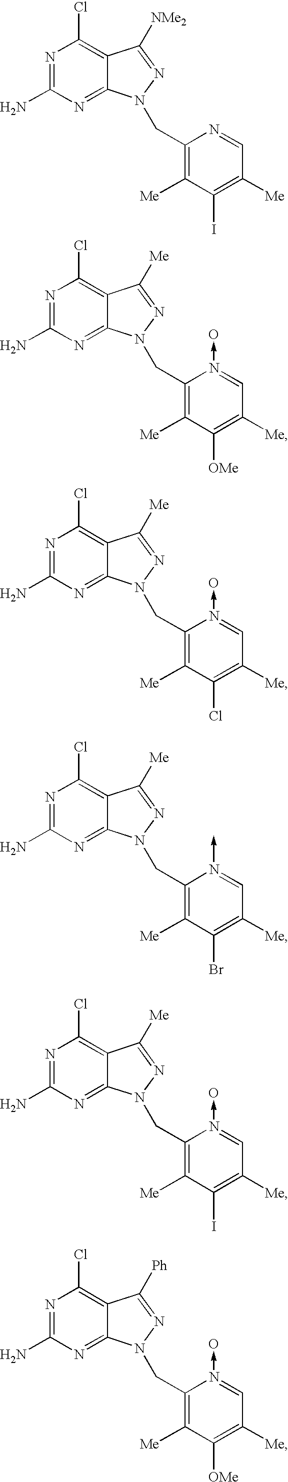

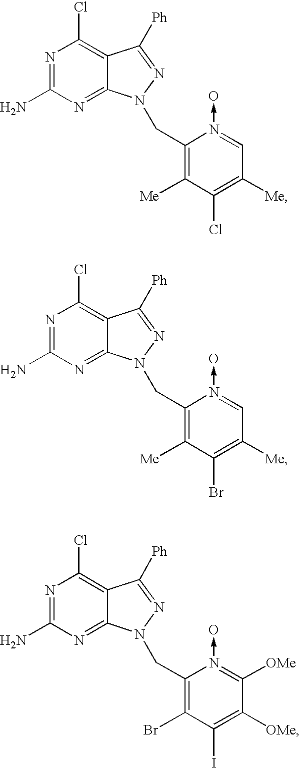

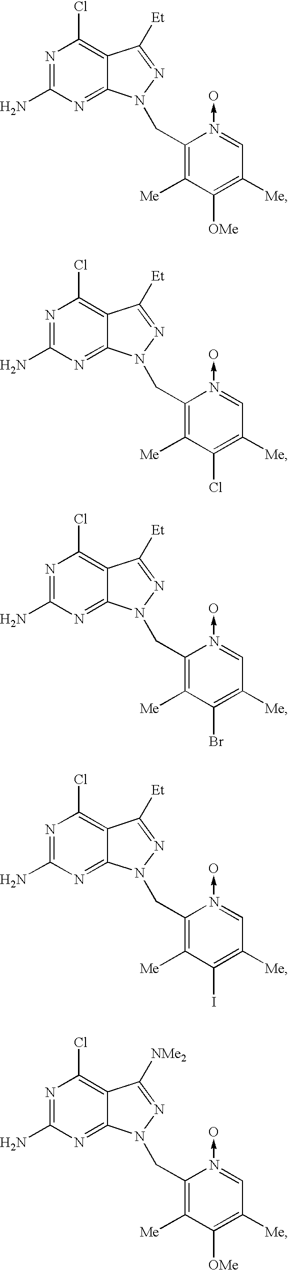

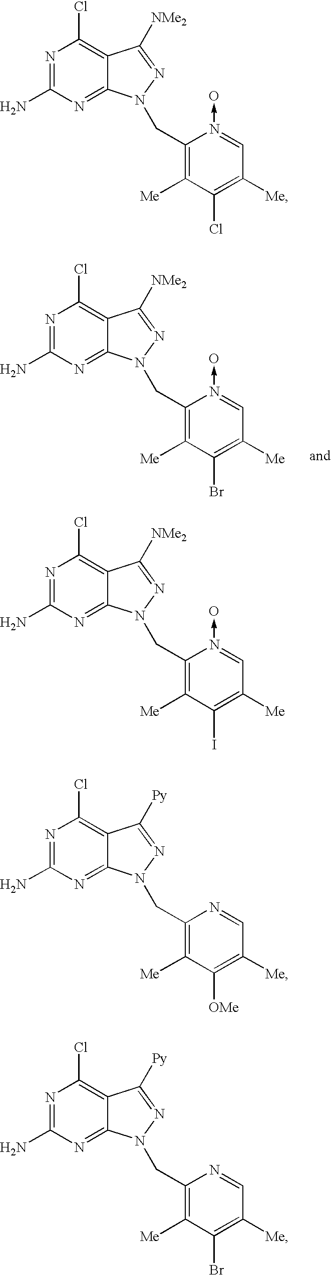

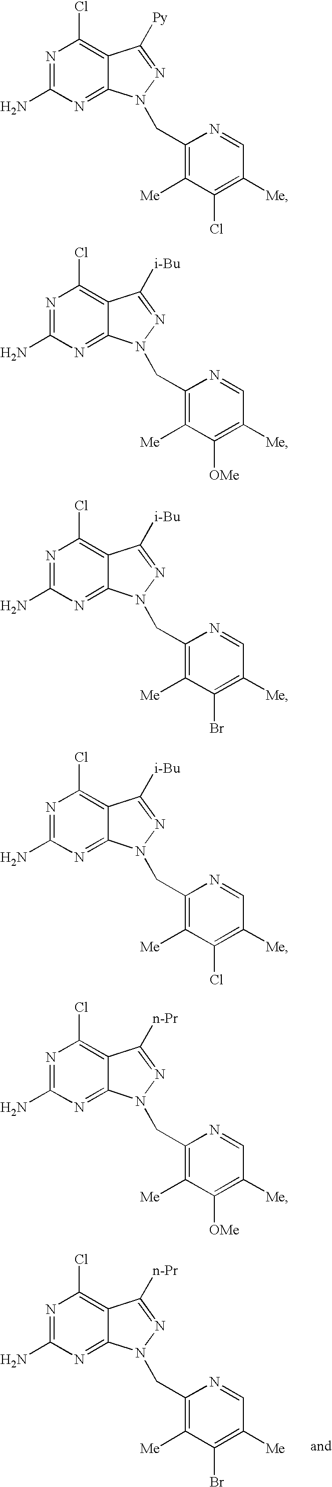

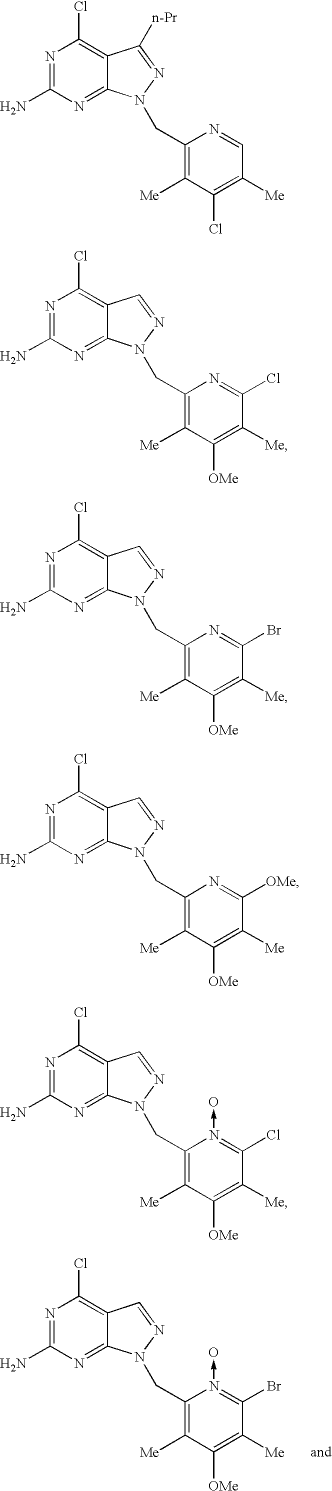

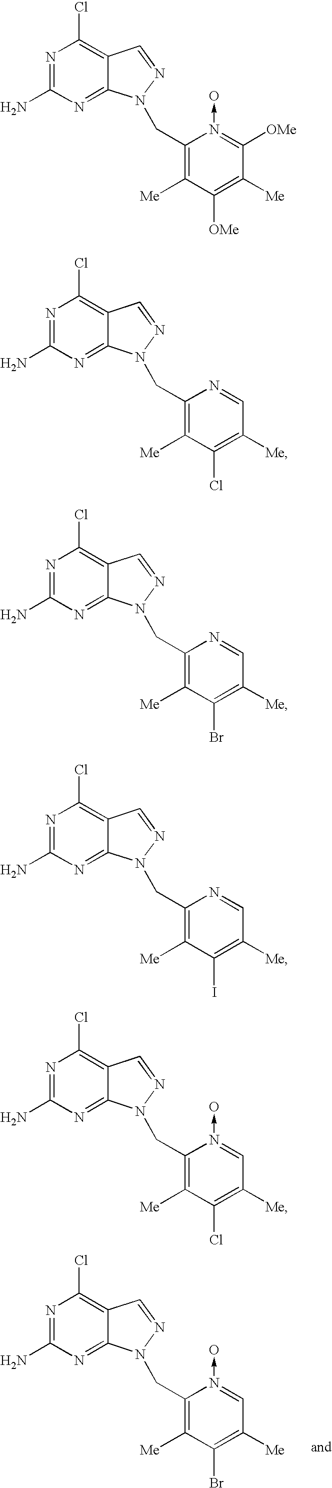

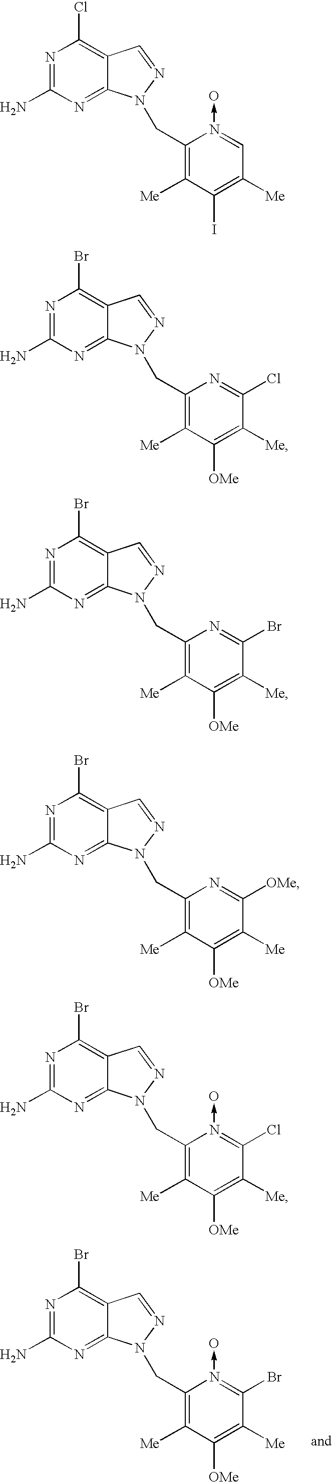

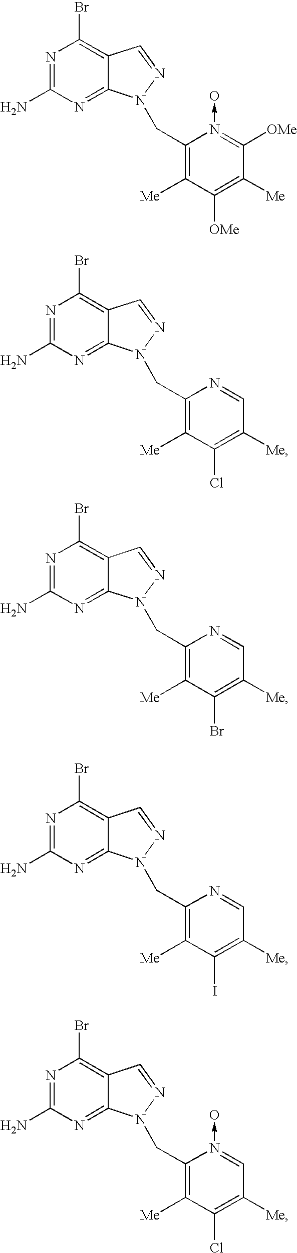

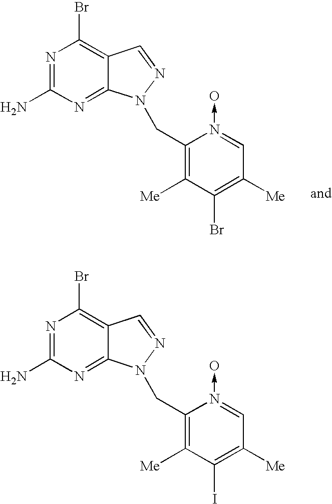

43. A method of treating an individual having an HSP90 mediated disorder comprising selecting a synthetic heterocyclic HSP 90 inhibitor wherein the activity of the HSP90 inhibitor is substantially independent of multi drug resistance, and administering to said individual a pharmaceutical composition comprising a pharmaceutically effective amount of said synthetic heterocyclic HSP90 inhibitor, wherein said selected synthetic heterocyclic HSP90 inhibitor is selected from the group below, or a polymorph, solvate, ester, tautomer, enantiomers, pharmaceutically acceptable salt or prodrug thereof:

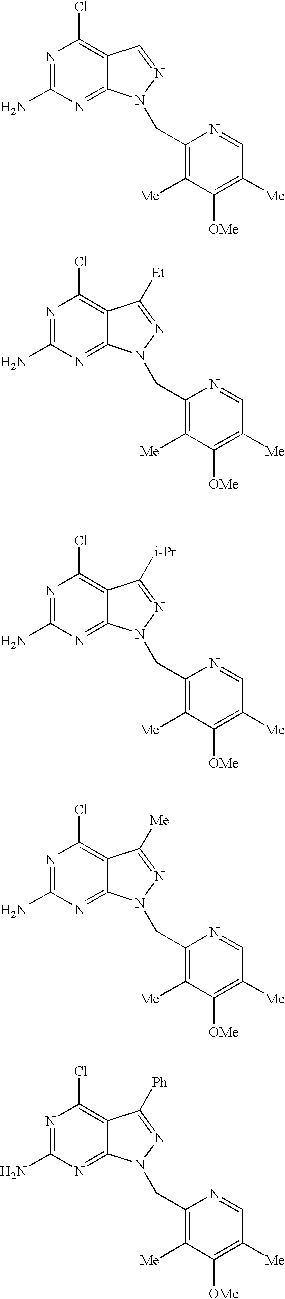

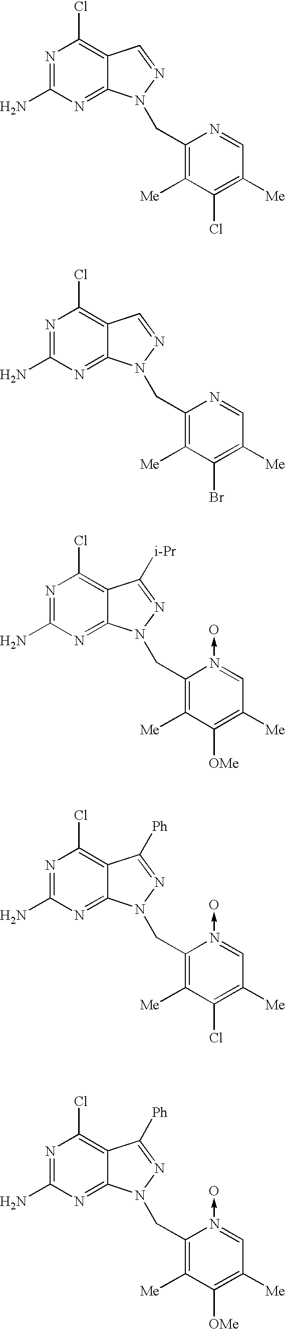

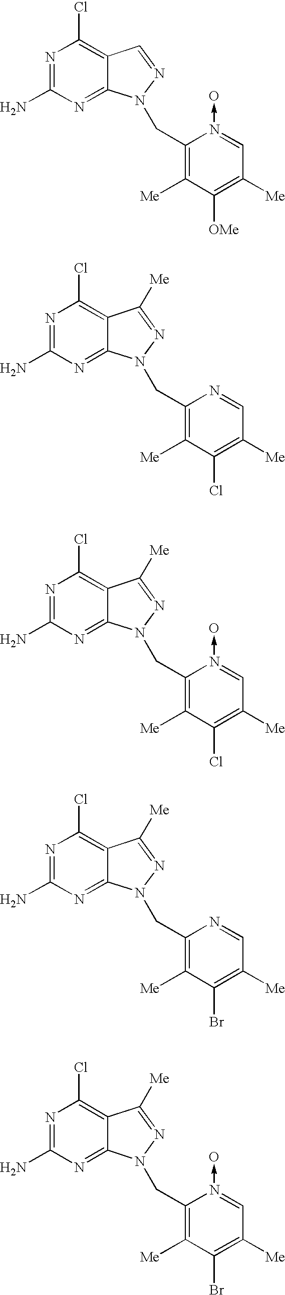

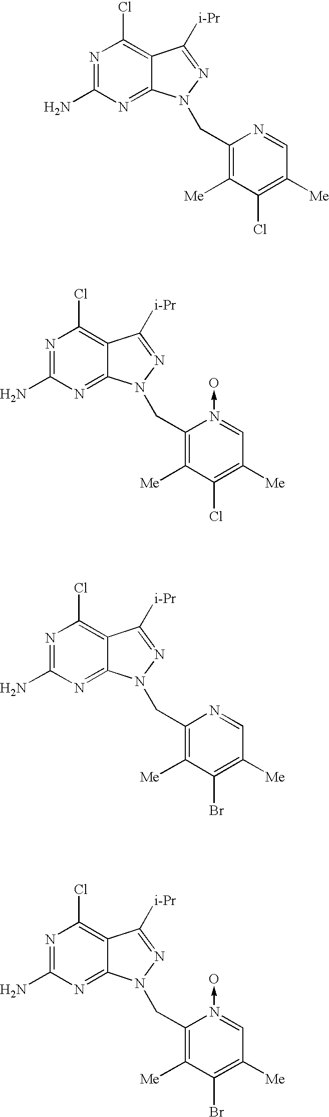

44. A method of treating an individual having an HSP90 mediated disorder comprising selecting a synthetic heterocyclic HSP 90 inhibitor wherein the activity of the HSP90 inhibitor is substantially independent of multi drug resistance, and administering to said individual a pharmaceutical composition comprising a pharmaceutically effective amount of said synthetic heterocyclic HSP90 inhibitor, wherein said selected synthetic heterocyclic HSP90 inhibitor is selected from the group below, or a polymorph, solvate, ester, tautomer, enantiomers, pharmaceutically acceptable salt or prodrug thereof:

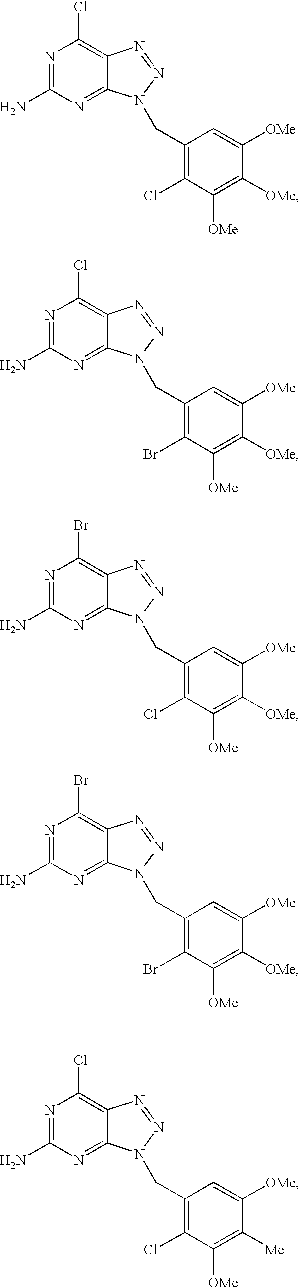

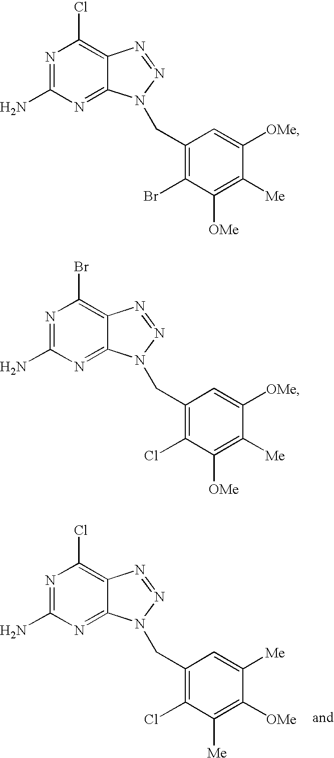

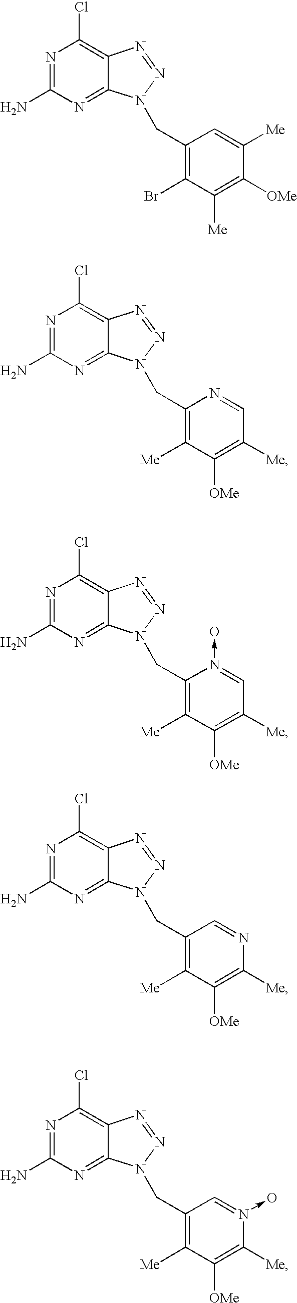

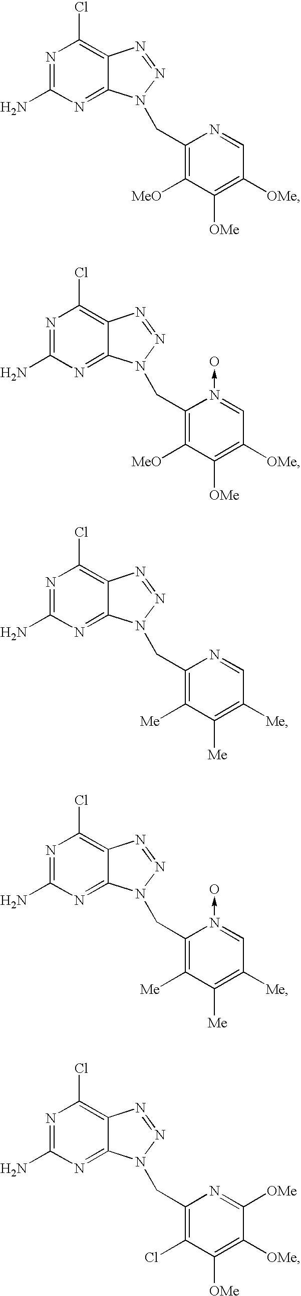

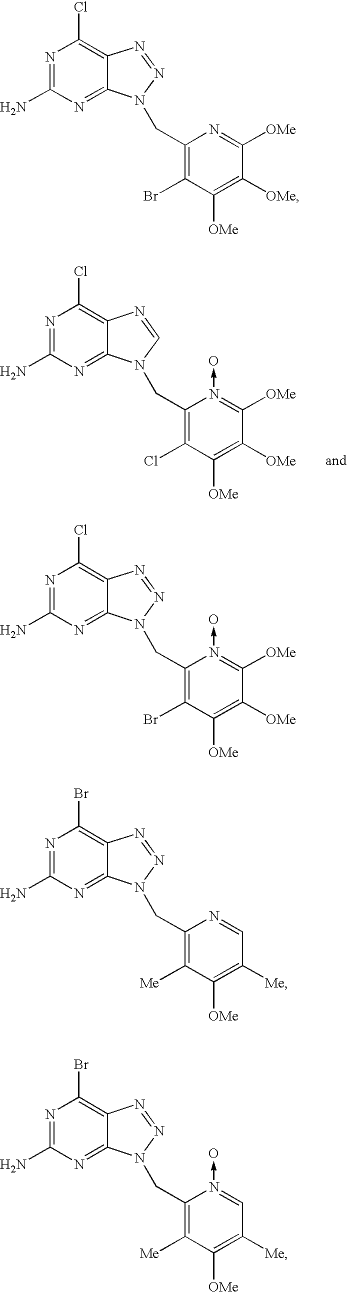

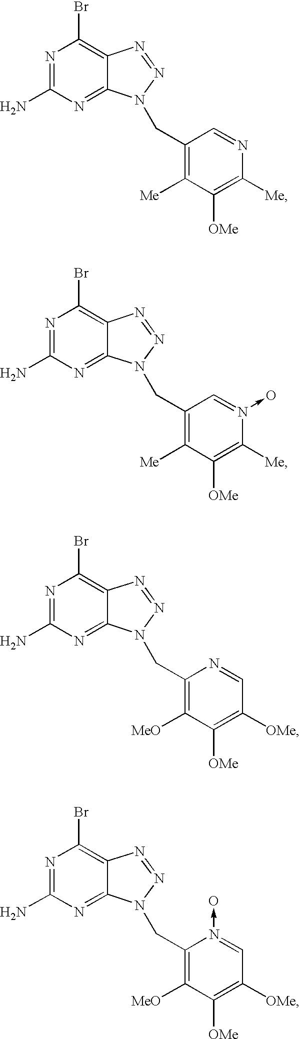

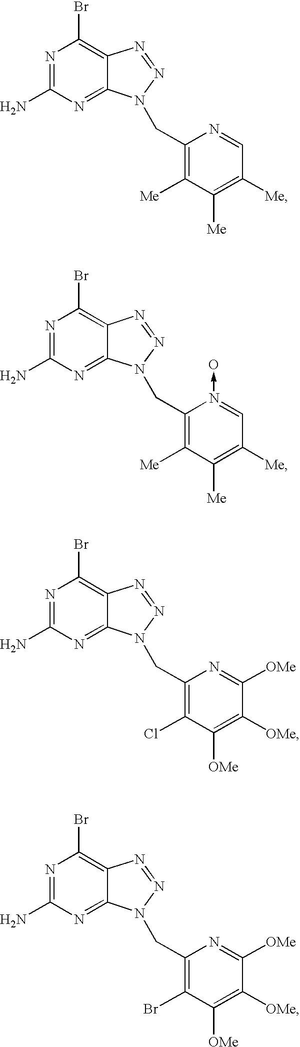

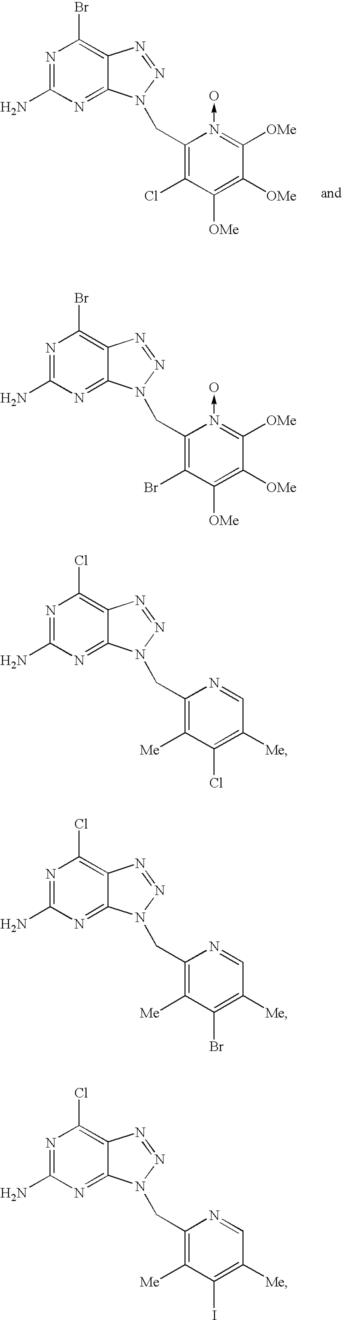

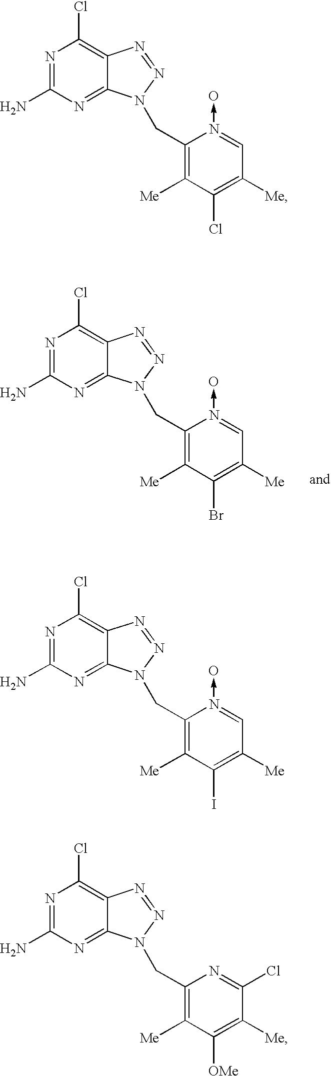

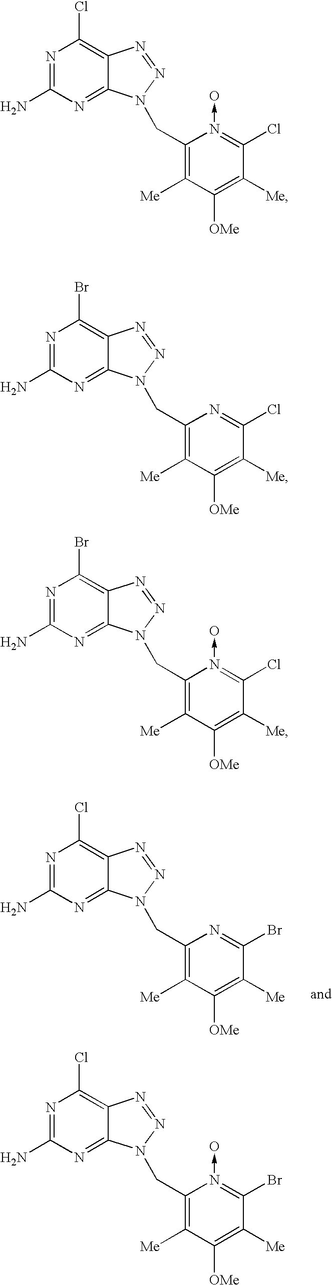

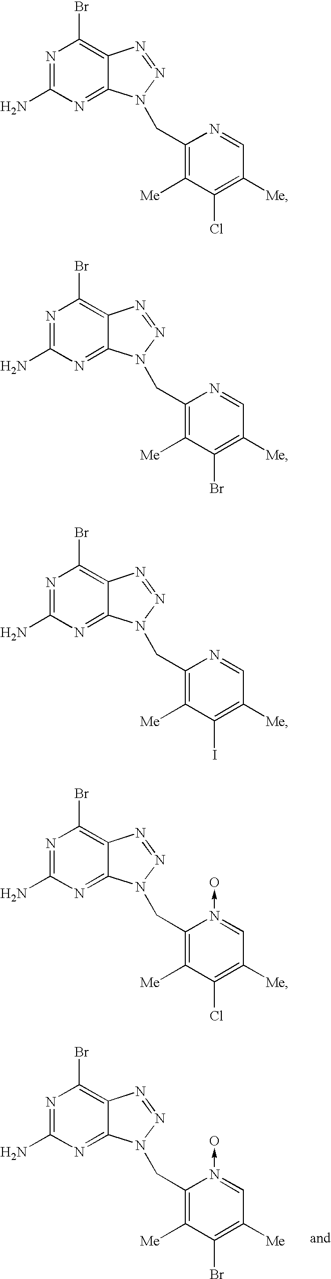

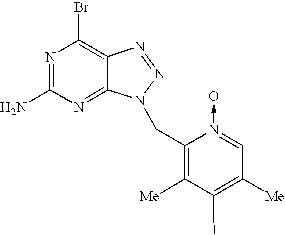

45. A method of treating an individual having an HSP90 mediated disorder comprising selecting a synthetic heterocyclic HSP 90 inhibitor wherein the activity of the HSP90 inhibitor is substantially independent of multi drug resistance, and administering to said individual a pharmaceutical composition comprising a pharmaceutically effective amount of said synthetic heterocyclic HSP90 inhibitor, wherein said selected synthetic heterocyclic HSP90 inhibitor is selected from the group below, or a polymorph, solvate, ester, tautomer, enantiomers, pharmaceutically acceptable salt or prodrug thereof:

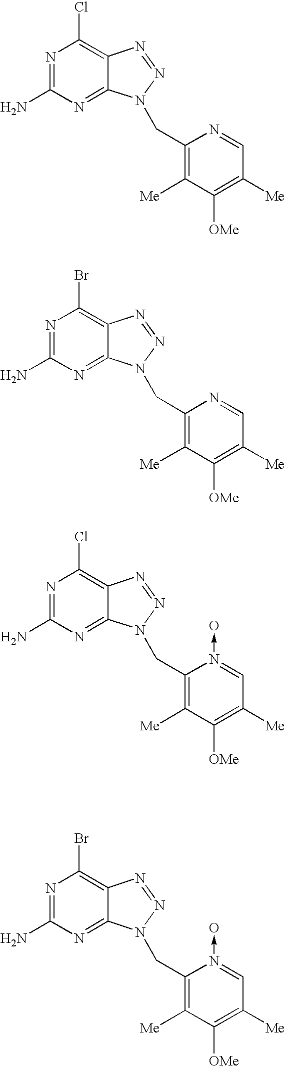

46. A method of treating an individual having an HSP90 mediated disorder comprising selecting a synthetic heterocyclic HSP 90 inhibitor wherein the activity of the HSP90 inhibitor is substantially independent of multi drug resistance, and administering to said individual a pharmaceutical composition comprising a pharmaceutically effective amount of said synthetic heterocyclic HSP90 inhibitor, wherein the activity of the HSP90 inhibitor is substantially independent of multi drug resistance and said selected synthetic heterocyclic HSP90 inhibitor is selected from the group below, or a polymorph, solvate, ester, tautomer, enantiomers, pharmaceutically acceptable salt or prodrug thereof:

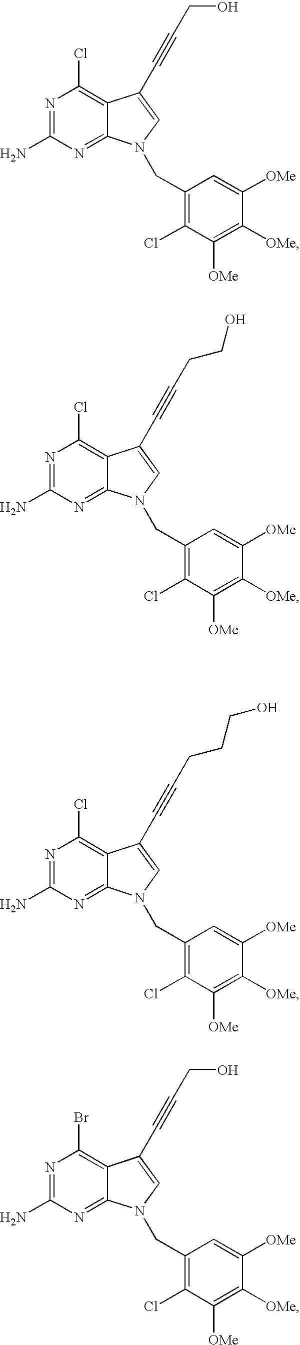

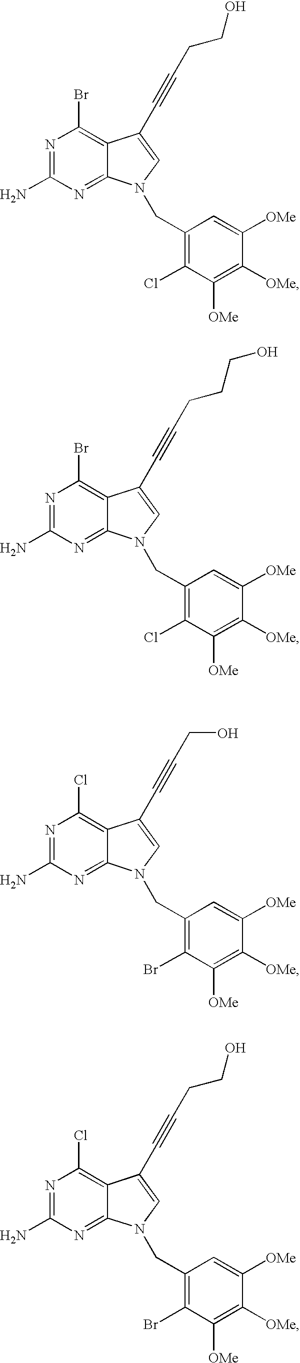

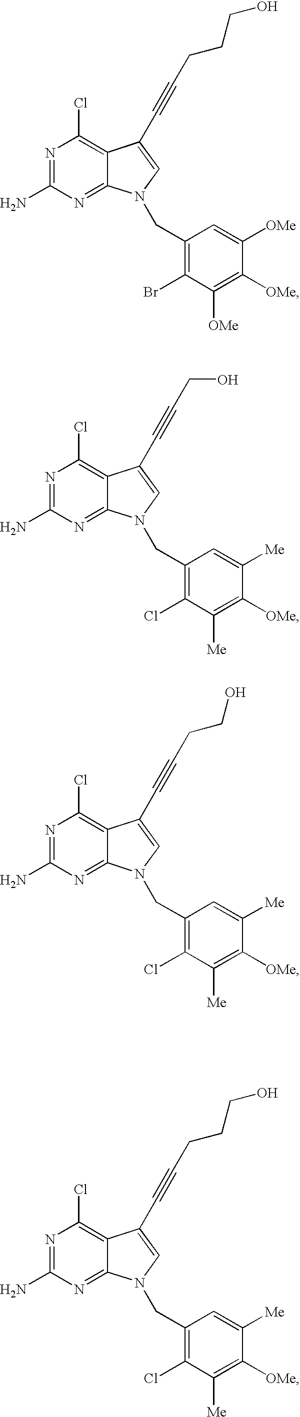

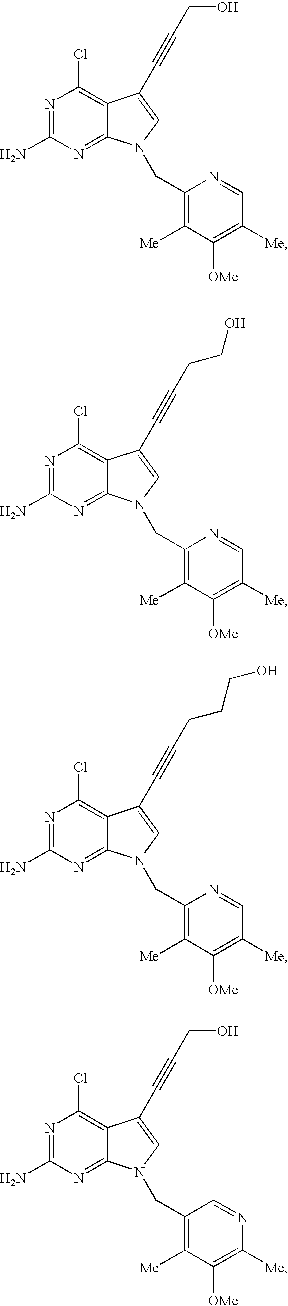

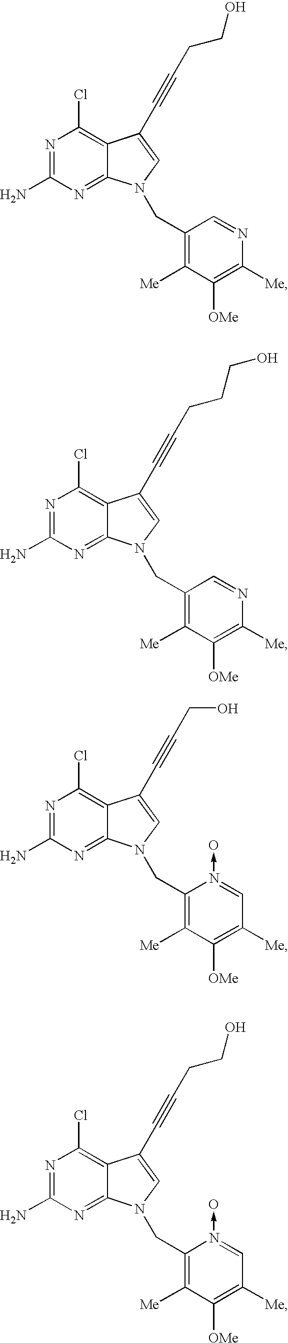

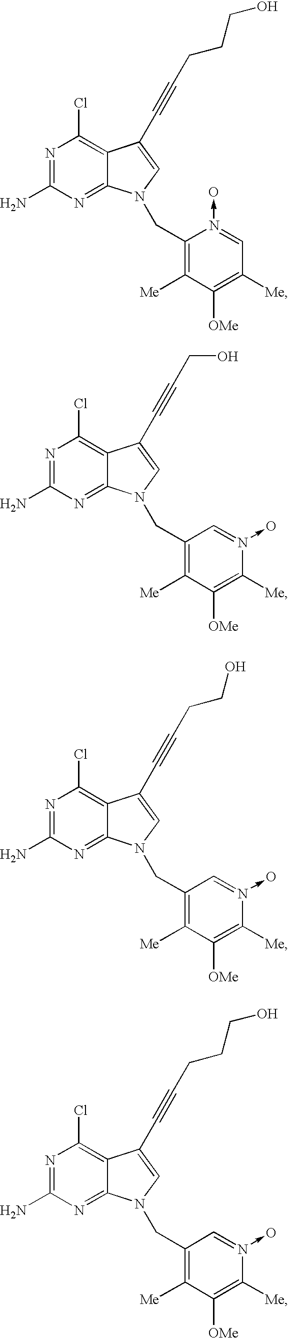

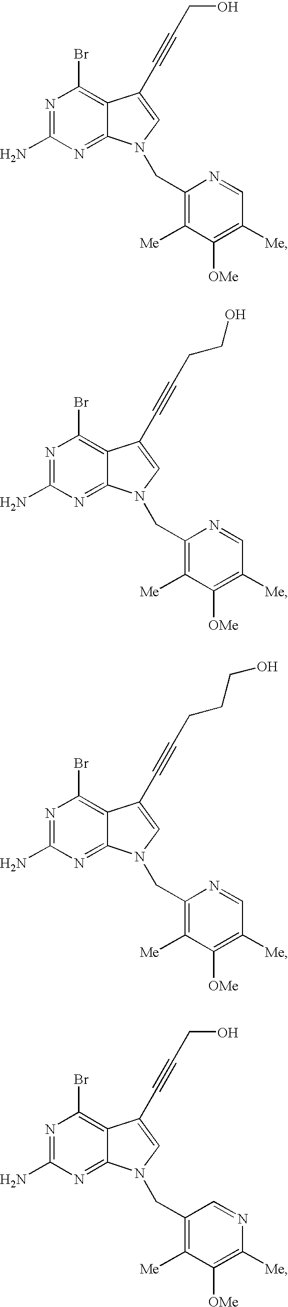

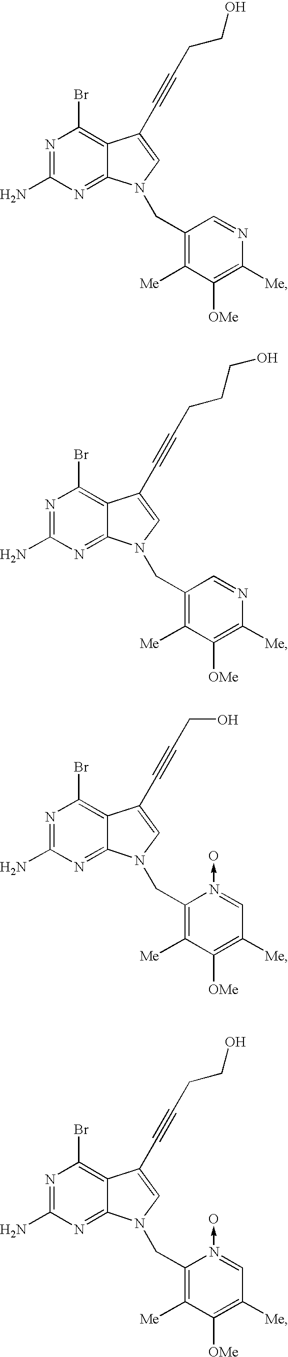

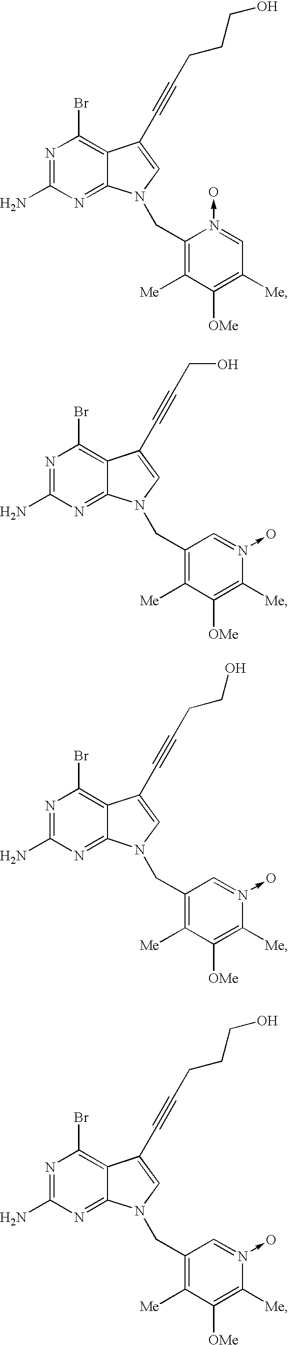

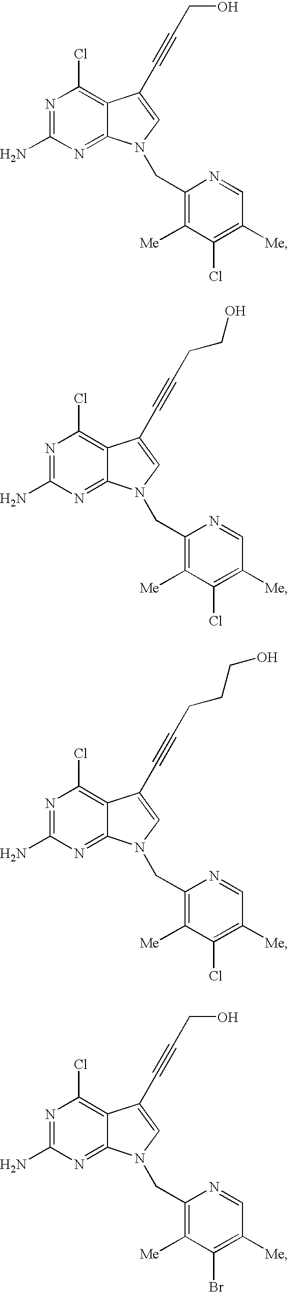

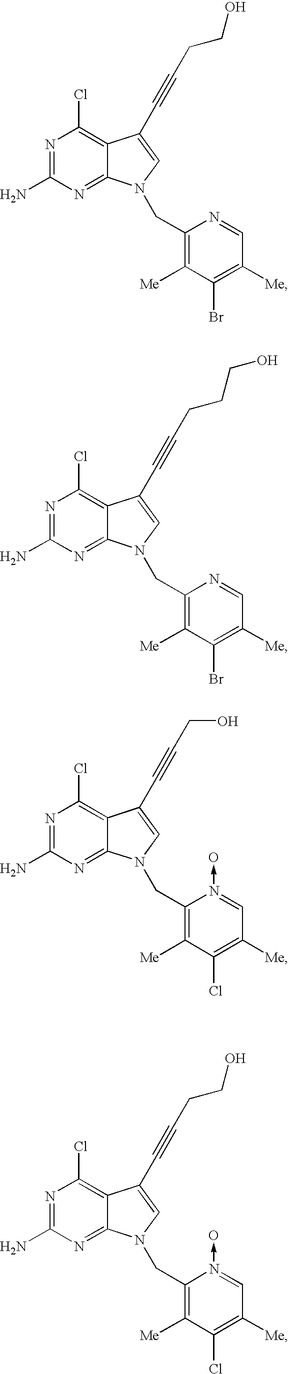

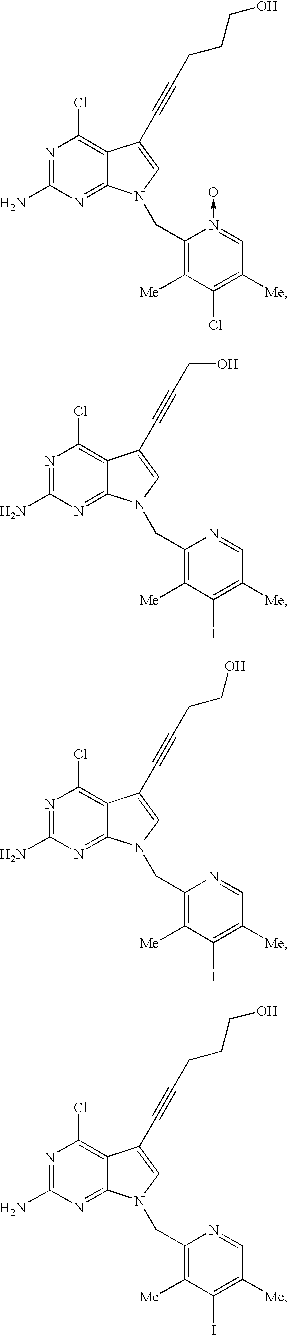

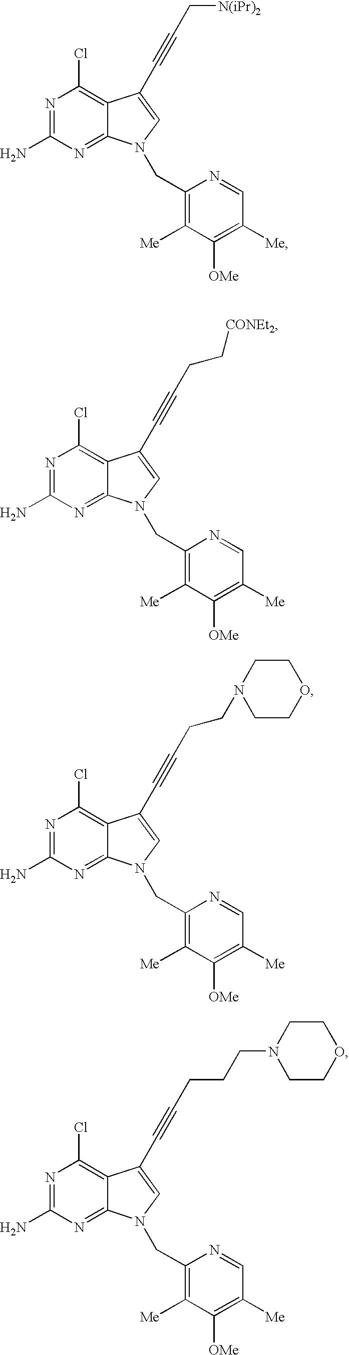

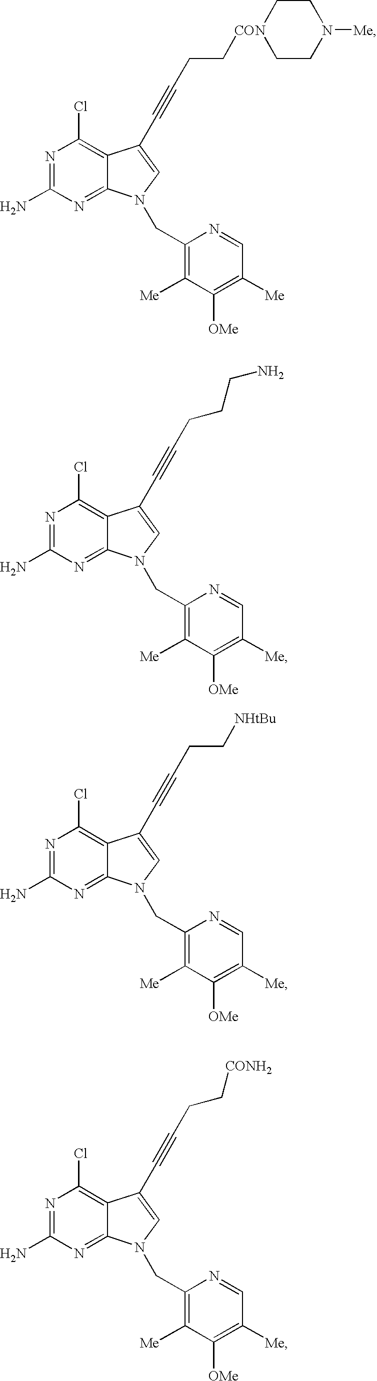

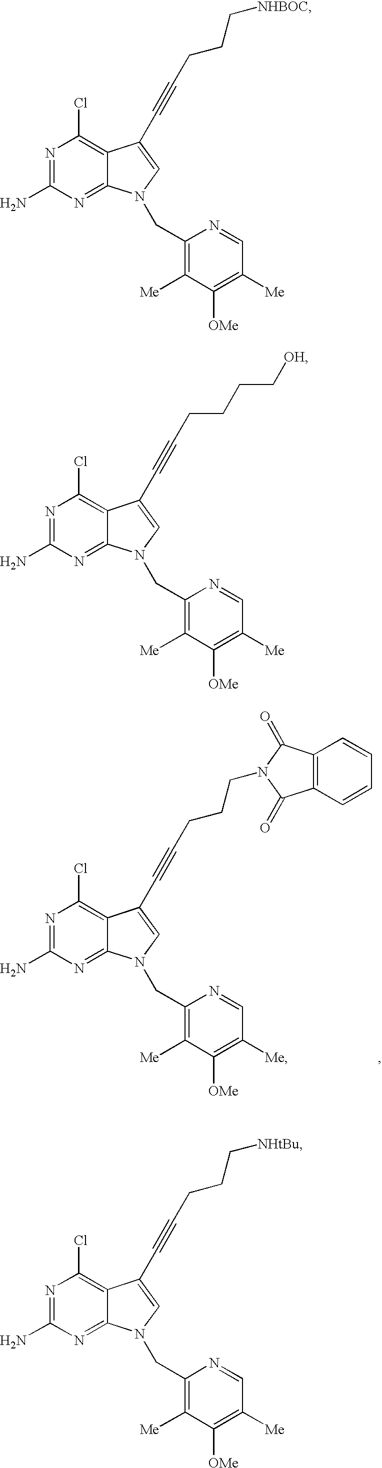

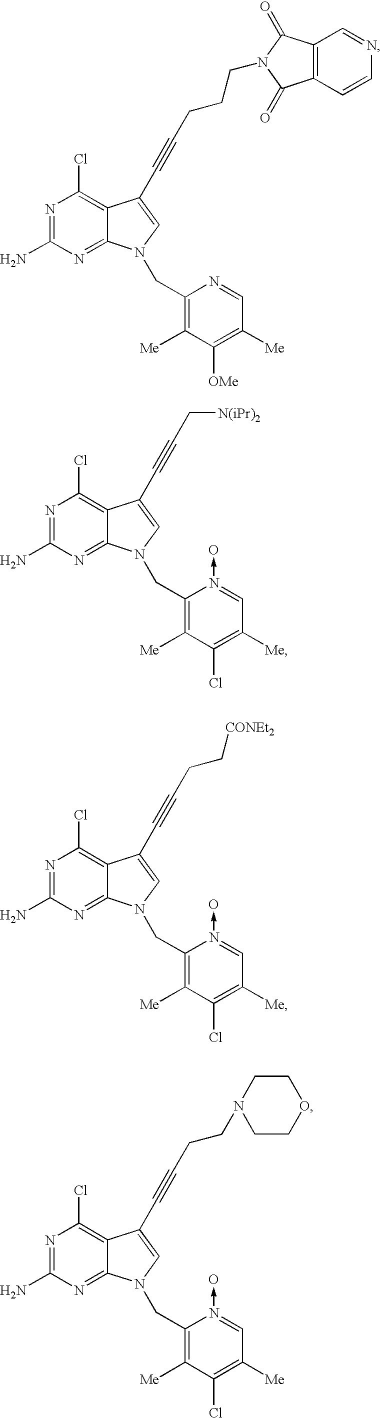

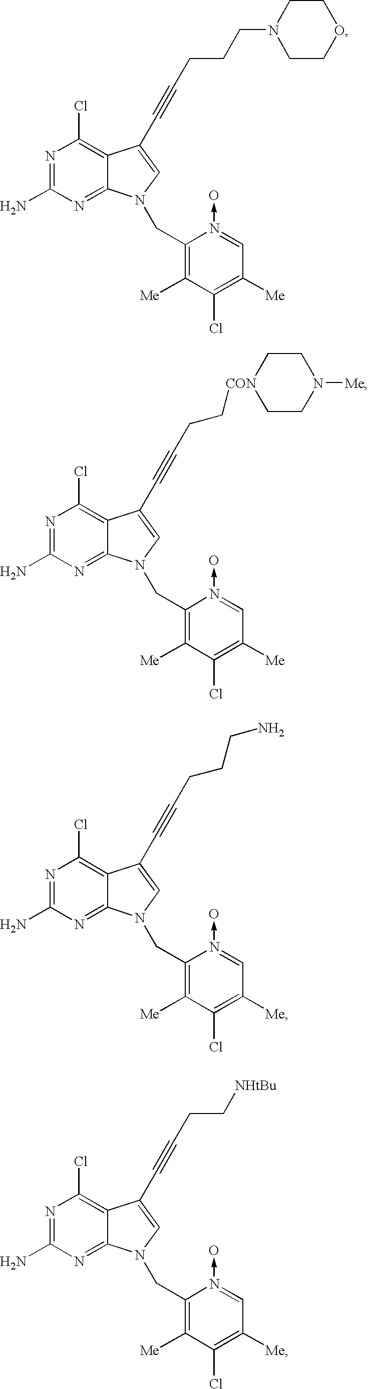

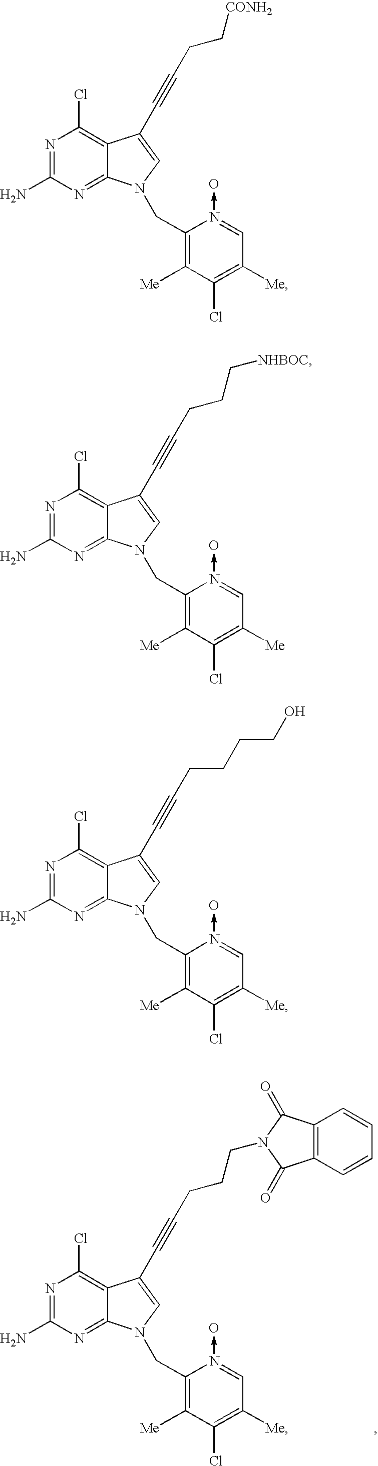

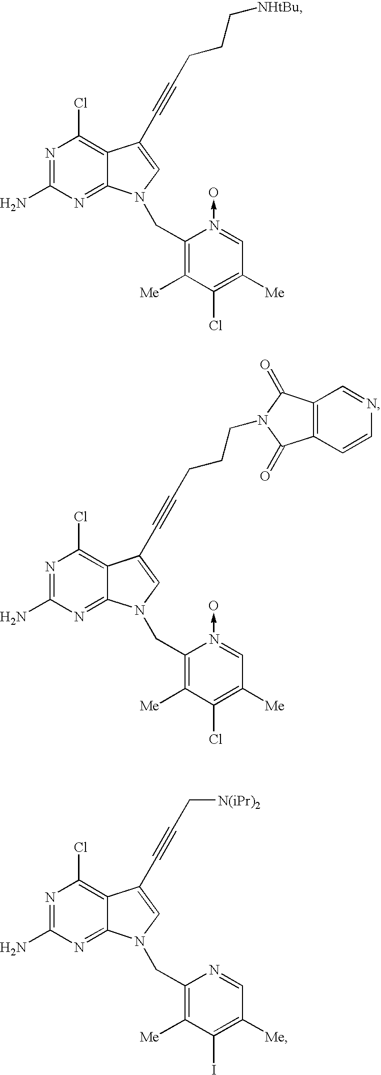

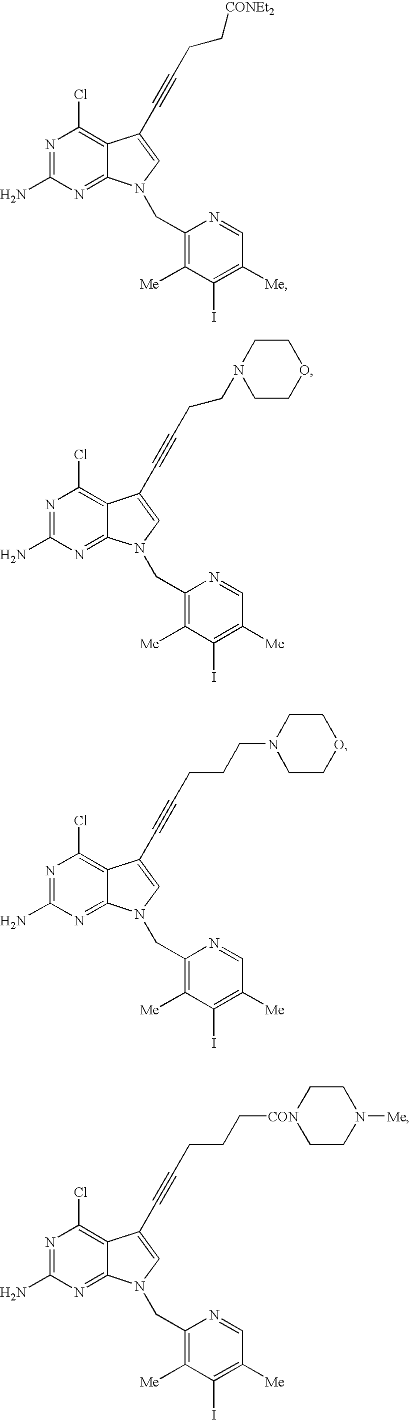

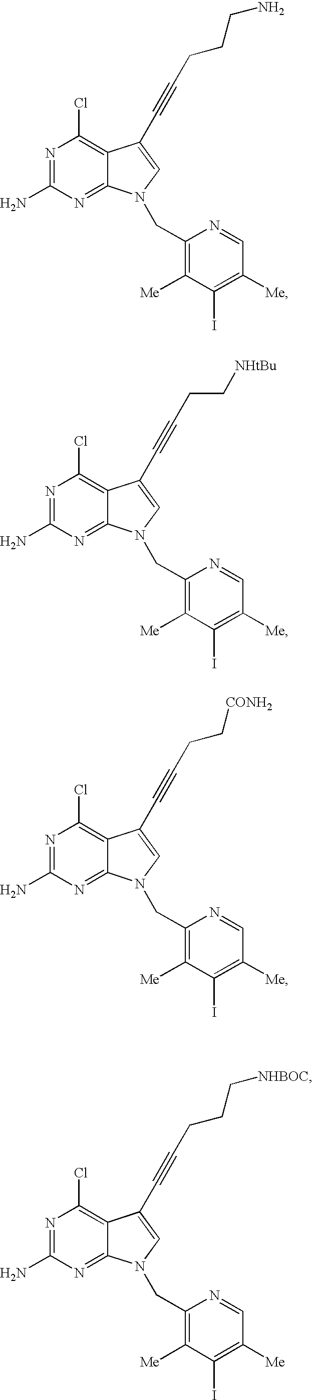

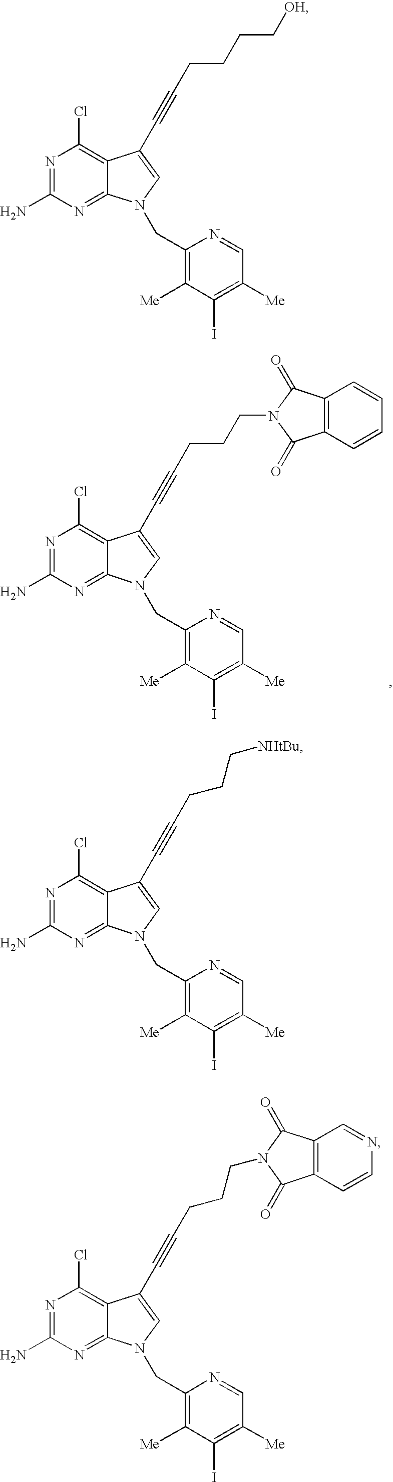

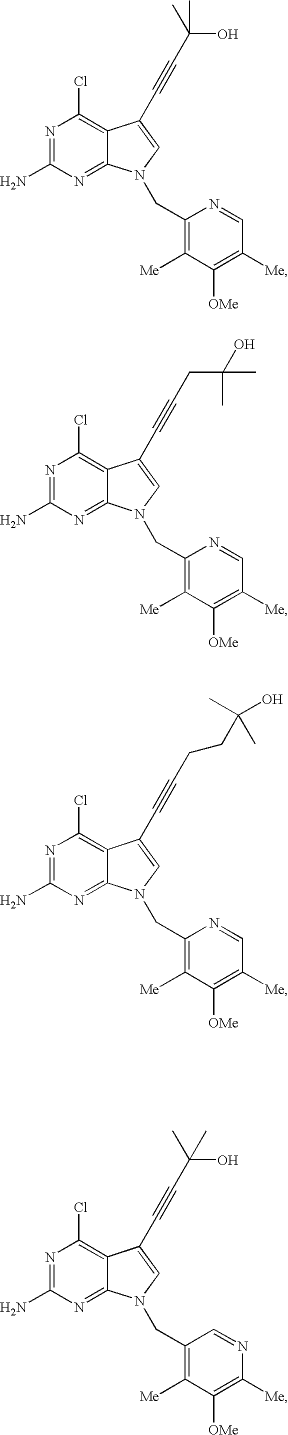

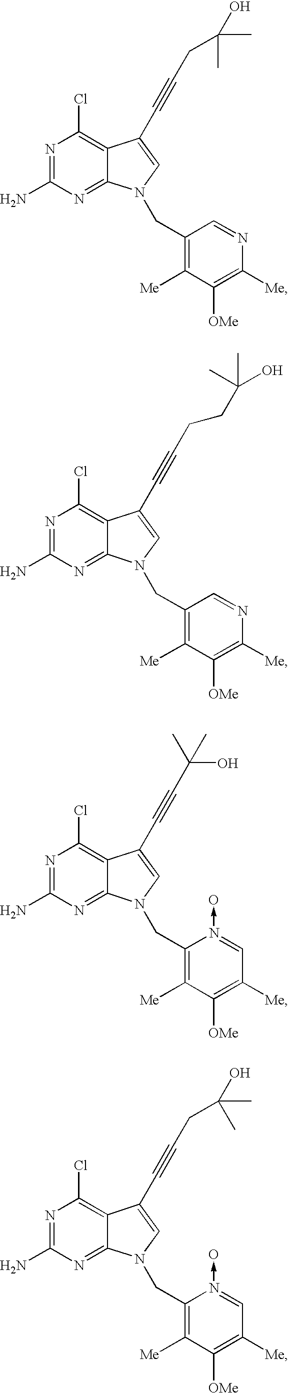

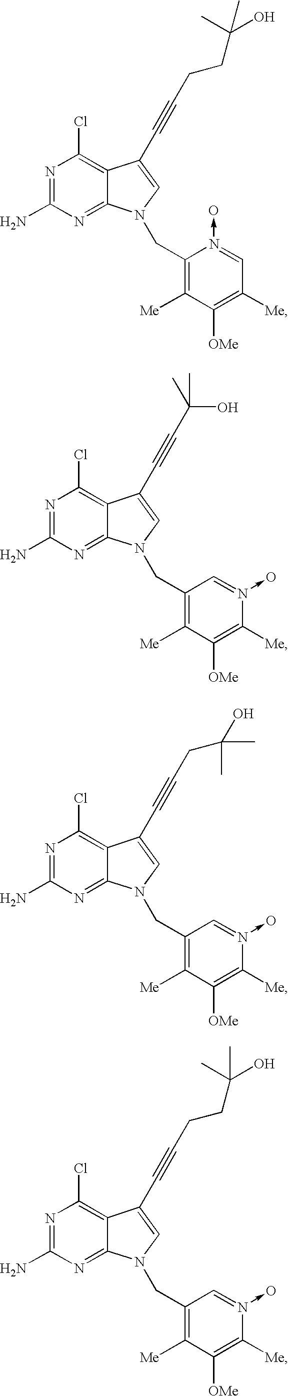

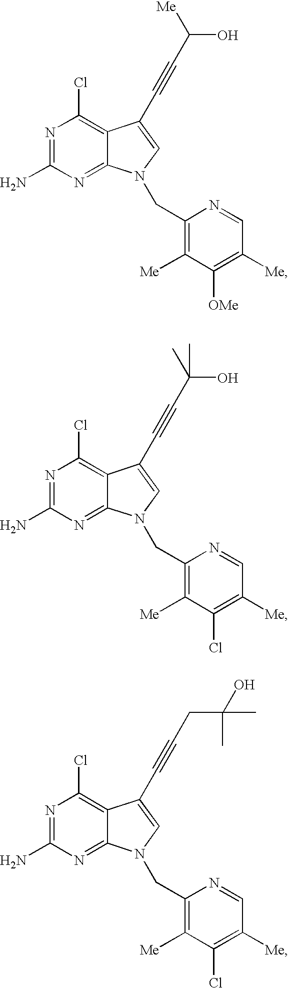

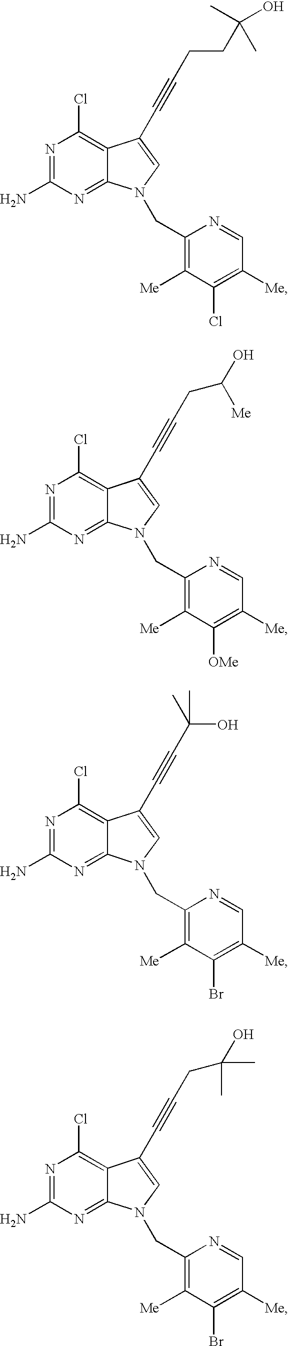

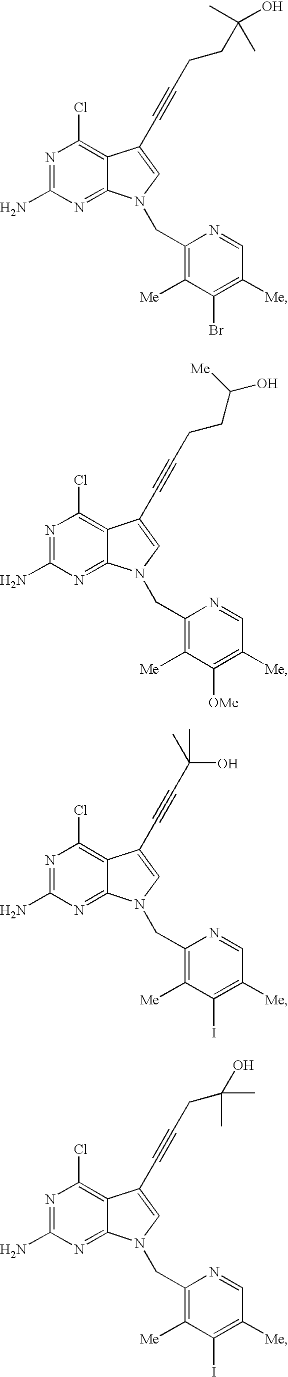

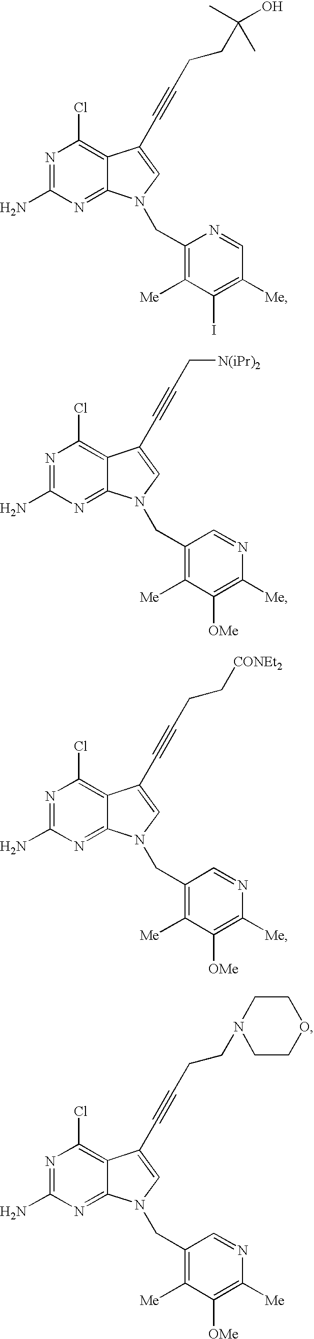

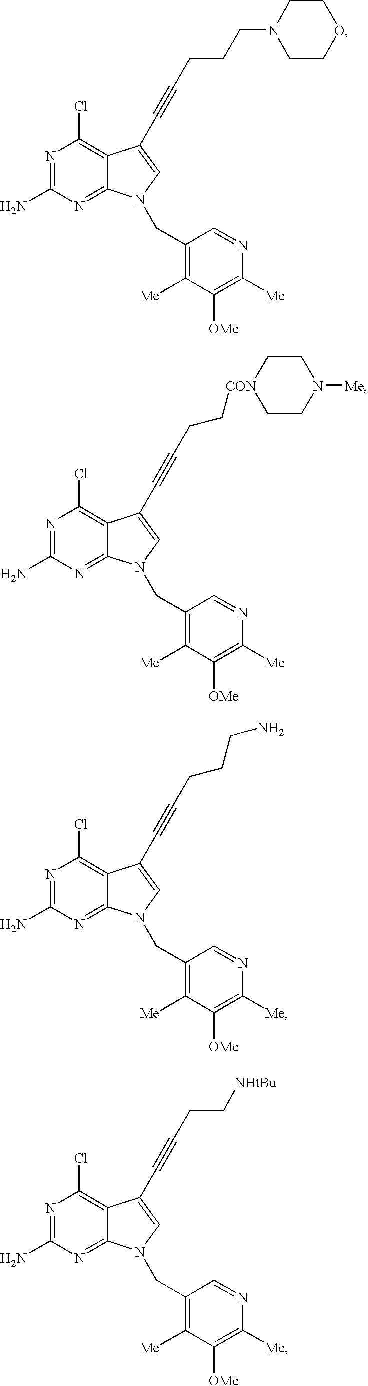

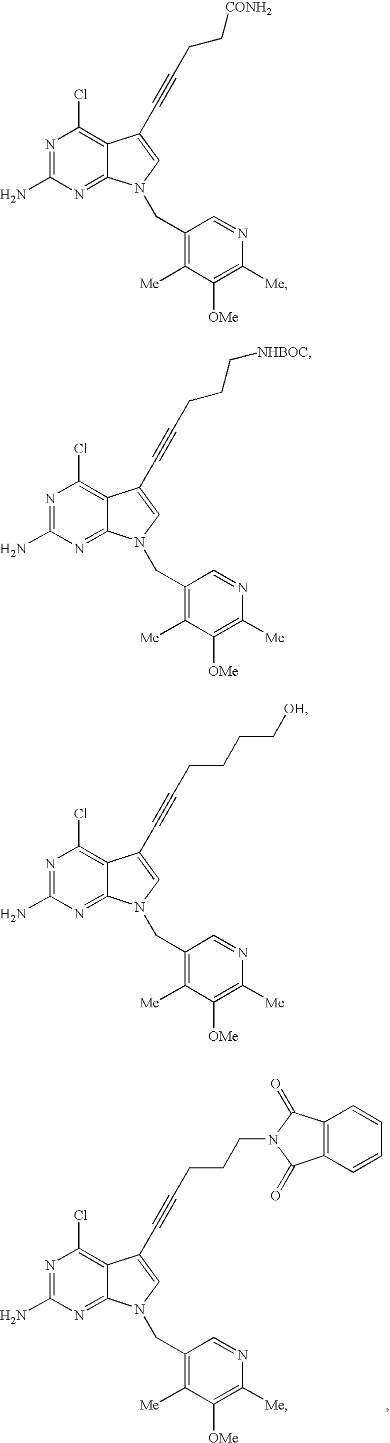

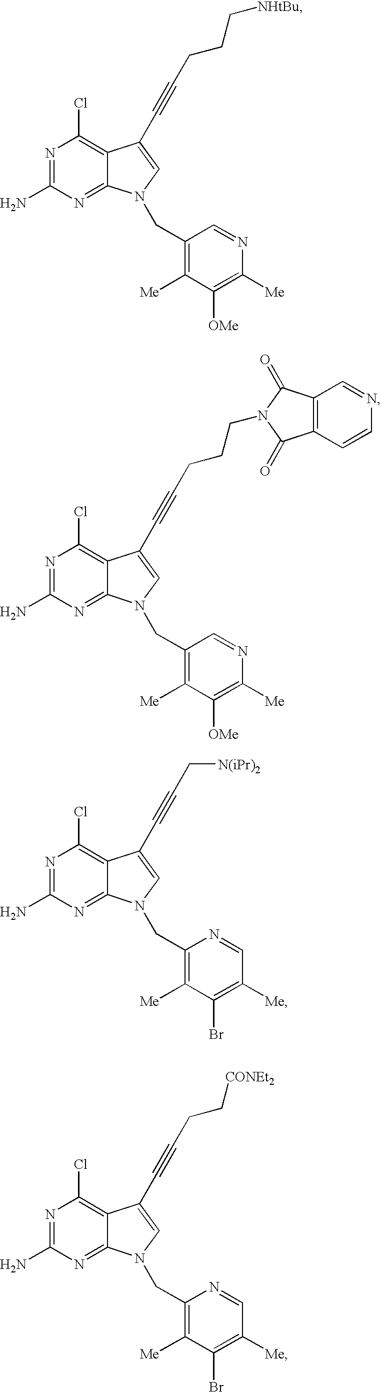

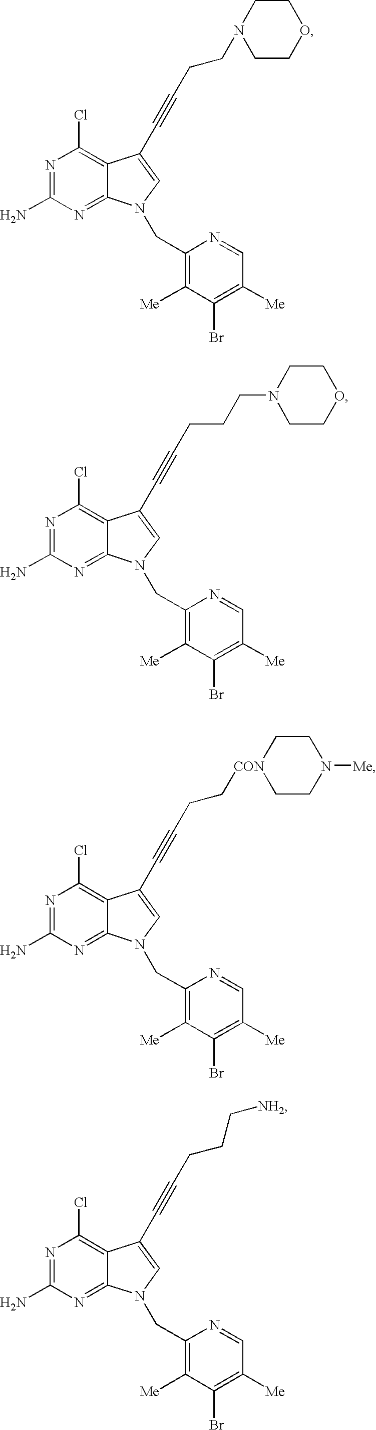

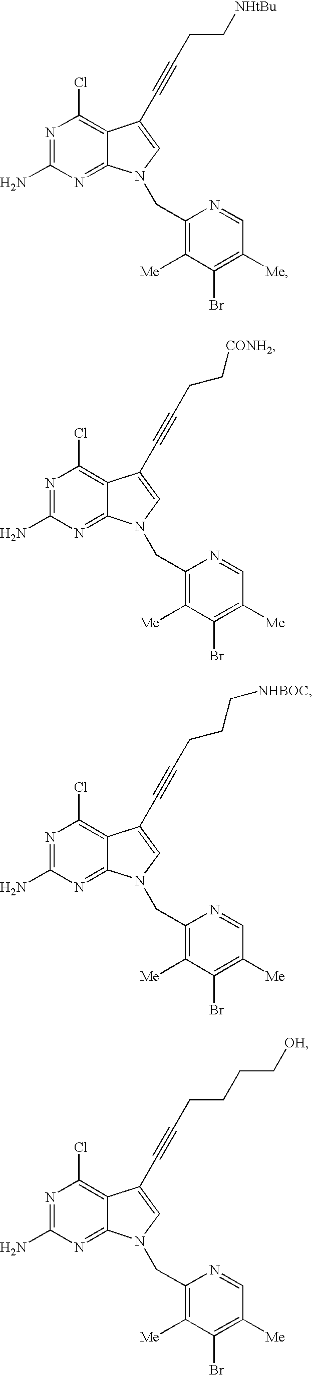

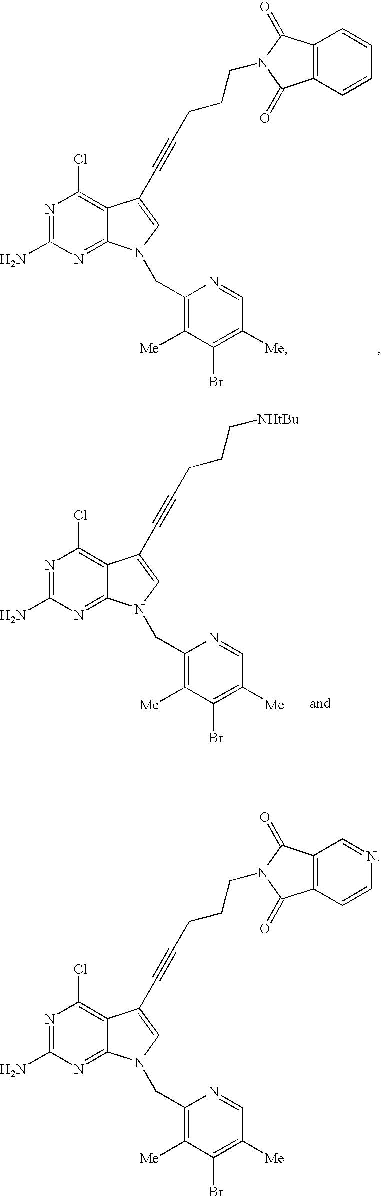

47. A method of treating an individual having an HSP90 mediated disorder comprising selecting a synthetic heterocyclic HSP 90 inhibitor wherein the activity of the HSP90 inhibitor is substantially independent of multi drug resistance, and administering to said individual a pharmaceutical composition comprising a pharmaceutically effective amount of said synthetic heterocyclic HSP90 inhibitor, wherein said selected synthetic heterocyclic HSP90 inhibitor is selected from the group below, or a polymorph, solvate, ester, tautomer, enantiomers, pharmaceutically acceptable salt or prodrug thereof:

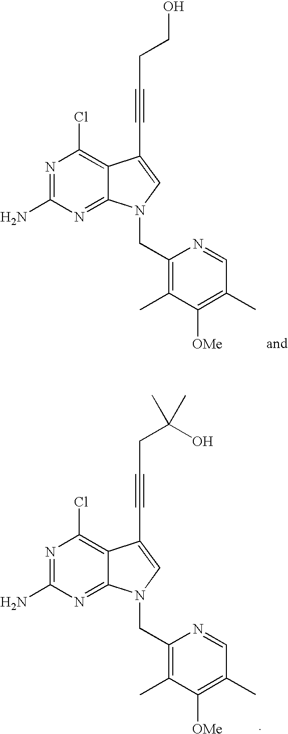

47. A method of treating an individual having an HSP90 mediated disorder comprising selecting a synthetic heterocyclic HSP 90 inhibitor wherein the activity of the HSP90 inhibitor is substantially independent of multi drug resistance, and administering to said individual a pharmaceutical composition comprising a pharmaceutically effective amount of said synthetic heterocyclic HSP90 inhibitor, wherein said selected synthetic heterocyclic HSP90 inhibitor is selected from the group below, or a polymorph, solvate, ester, tautomer, enantiomers, pharmaceutically acceptable salt or prodrug thereof: 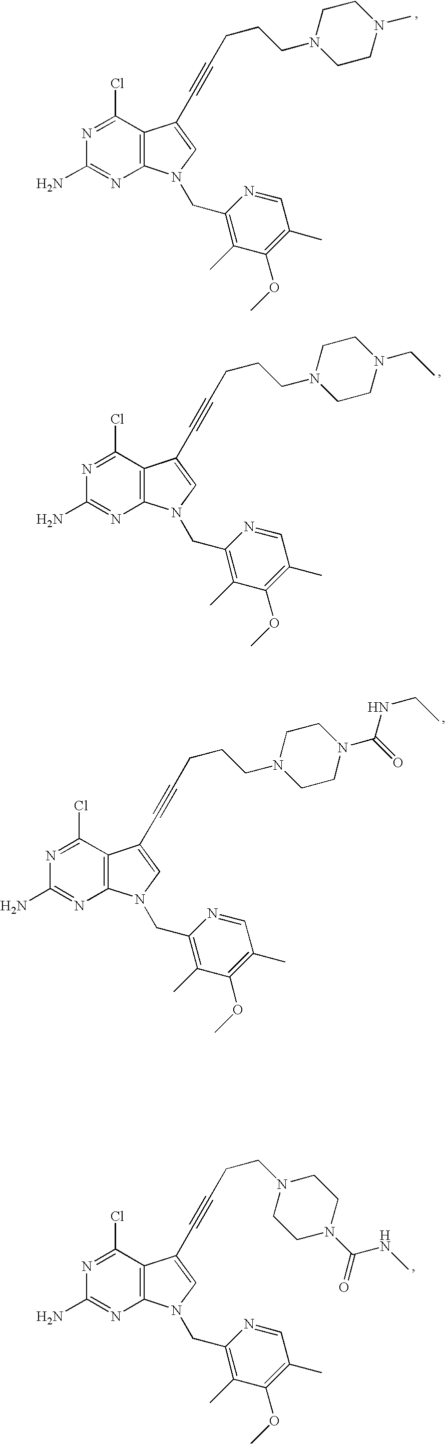 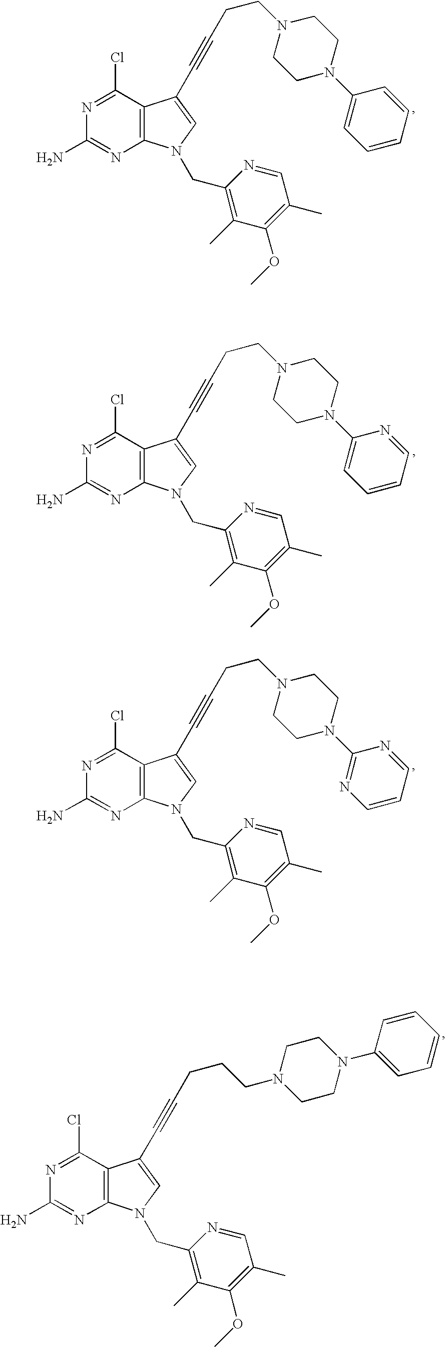 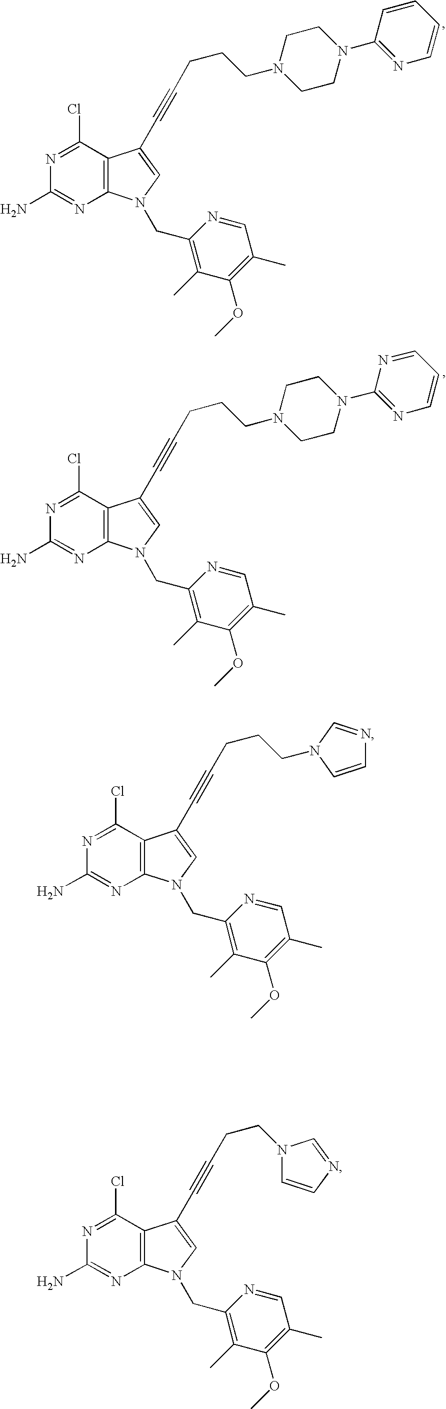 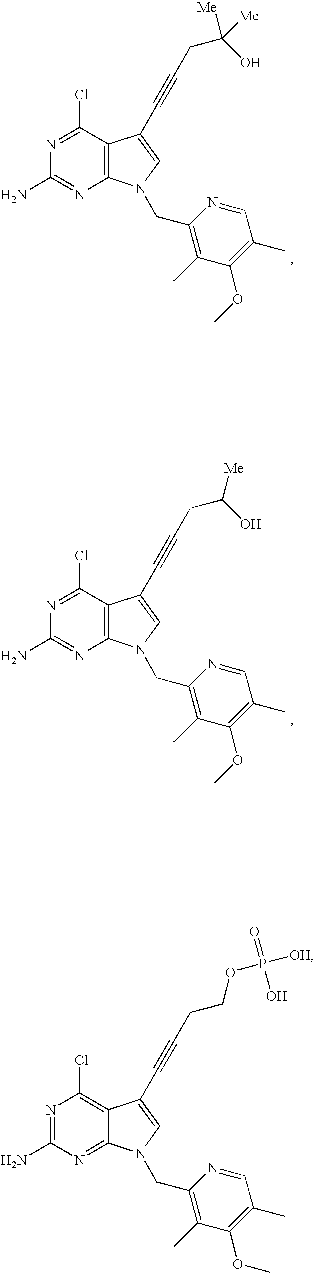 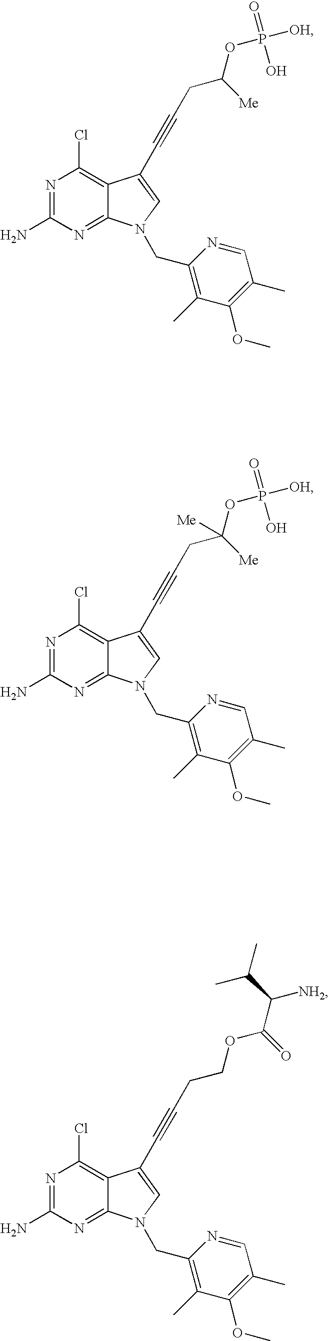 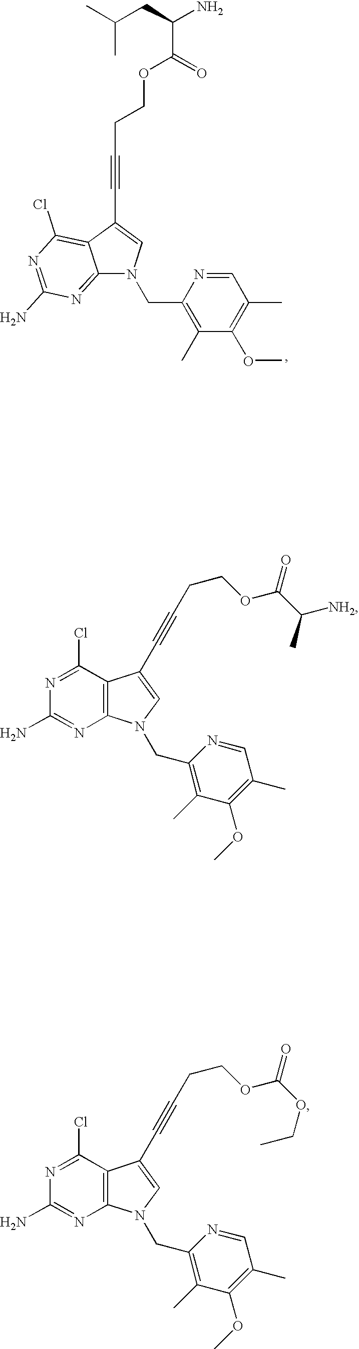 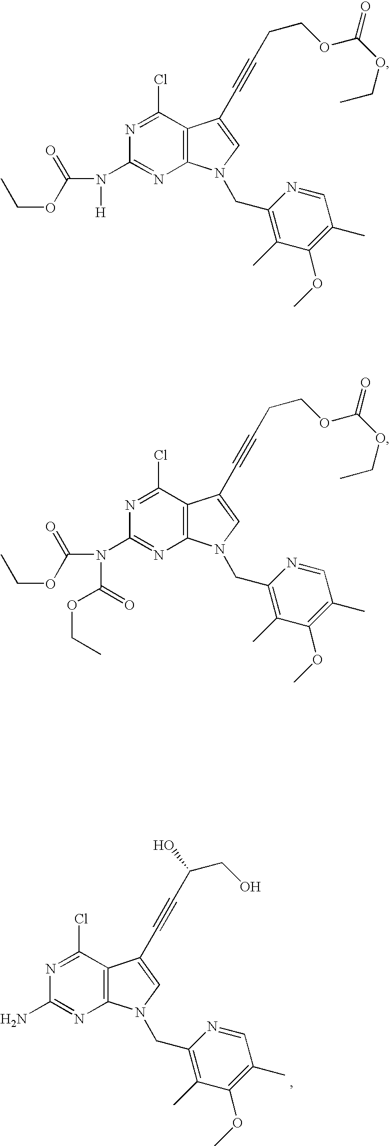 [chemical structure]
